SEQUENCE LISTING (62 sequences)

SEQ ID NO: 1 - protein, 11 aa, Artificial Sequence
  Antibody sequence
RSSQGIRSSLA

SEQ ID NO: 2 - protein, 7 aa, Artificial Sequence
  Antibody sequence
GASSLEG

SEQ ID NO: 3 - protein, 9 aa, Artificial Sequence
  Antibody sequence
QQLNSFPST

SEQ ID NO: 4 - protein, 9 aa, Artificial Sequence
  Antibody sequence
QQLASFPST

SEQ ID NO: 5 - protein, 7 aa, Artificial Sequence
  Antibody sequence
GASSLEV

SEQ ID NO: 6 - protein, 7 aa, Artificial Sequence
  Antibody sequence
GGSSLEG

SEQ ID NO: 7 - protein, 7 aa, Artificial Sequence
  Antibody sequence
GASSLLG

SEQ ID NO: 8 - protein, 10 aa, Artificial Sequence
  Antibody sequence
GFTFGSYEMN

SEQ ID NO: 9 - protein, 10 aa, Artificial Sequence
  Antibody sequence
YISSSGETTD

SEQ ID NO: 10 - protein, 18 aa, Artificial Sequence
  Antibody sequence
DVFFFDSSGDNGAYGMDV

SEQ ID NO: 11 - protein, 18 aa, Artificial Sequence
  Antibody sequence
DVFFFSPSGDNGAYGMDV

SEQ ID NO: 12 - protein, 10 aa, Artificial Sequence
  Antibody sequence
GFTAGSYEMN

SEQ ID NO: 13 - protein, 18 aa, Artificial Sequence
  Antibody sequence
DVFFFDSSGPRGAYGMDV

SEQ ID NO: 14 - protein, 10 aa, Artificial Sequence
  Antibody sequence
GFTFGSYEMD

SEQ ID NO: 15 - protein, 18 aa, Artificial Sequence
  Antibody sequence
DVFFFDSSGSPGAYGMDV

SEQ ID NO: 16 - protein, 18 aa, Artificial Sequence
  Antibody sequence
DVFFFDSSGDNHAYGMDV

SEQ ID NO: 17 - protein, 18 aa, Artificial Sequence
  Antibody sequence
DVFFFDSSGPPGAYGMDV

SEQ ID NO: 18 - protein, 18 aa, Artificial Sequence
  Antibody sequence
DVFFFDSSRPNGAYGMDV

SEQ ID NO: 19 - protein, 18 aa, Artificial Sequence
  Antibody sequence
DVFFFDSSGFPGAYGMDV

SEQ ID NO: 20 - protein, 10 aa, Artificial Sequence
  Antibody sequence
GFTPGSYEMN

SEQ ID NO: 21 - protein, 10 aa, Artificial Sequence
  Antibody sequence
YISSAGETTD

SEQ ID NO: 22 - DNA, 321 bp, Artificial Sequence
  Antibody sequence
gacatccagttgacccagtctccatcctccctgactgcatctgtcggagacagagtcaccattacttgccggtcaagtcagggcattcgcagttctttagcctggtatcaacagaaaccagggaaacctcctaagctcctgatctatggtgcctccagtttggaaggtggggtcccatcaaggttcagcggcagtggatctgggacagatttcactctcaccatcagtagtctgcagcctgaagattttgcaacttattactgtcagcagctcaatagtttcccgtccacttttggccaggggaccaagctggaaatcaaa

SEQ ID NO: 23 - protein, 107 aa, Artificial Sequence
  Antibody sequence
DIQLTQSPSSLTASVGDRVTITCRSSQGIRSSLAWYQQKPGKPPKLLIYGASSLEGGVPSRFSGSGSGTDFTLTISSLQPEDFATYYCQQLNSFPSTFGQGTKLEIK

SEQ ID NO: 24 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtgacaatggggcctacggtatggacgtctggggccaagggacaatggtcaccgtctcatca

SEQ ID NO: 25 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSS

SEQ ID NO: 26 - protein, 214 aa, Artificial Sequence
  Antibody sequence
DIQLTQSPSSLTASVGDRVTITCRSSQGIRSSLAWYQQKPGKPPKLLIYGASSLEGGVPSRFSGSGSGTDFTLTISSLQPEDFATYYCQQLNSFPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 457 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 321 bp, Artificial Sequence
  Antibody sequence
gacatccagttgacccagtctccatcctccctgactgcatctgtcggagacagagtcaccattacttgccggtcaagtcagggcattcgcagttctttagcctggtatcaacagaaaccagggaaacctcctaagctcctgatctatggtgcctccagtttggaaggtggggtcccatcaaggttcagcggcagtggatctgggacagatttcactctcaccatcagtagtctgcagcctgaagattttgcaacttattactgtcagcagcttgctagtttcccgtccacttttggccaggggaccaagctggaaatcaaa

SEQ ID NO: 29 - protein, 107 aa, Artificial Sequence
  Antibody sequence
DIQLTQSPSSLTASVGDRVTITCRSSQGIRSSLAWYQQKPGKPPKLLIYGASSLEGGVPSRFSGSGSGTDFTLTISSLQPEDFATYYCQQLASFPSTFGQGTKLEIK

SEQ ID NO: 30 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgttttttttttcgccgagtggtgacaatggggcctacggtatggacgtctggggccaagggacaatggtcaccgtctcatca

SEQ ID NO: 31 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFSPSGDNGAYGMDVWGQGTMVTVSS

SEQ ID NO: 32 - DNA, 321 bp, Artificial Sequence
  Antibody sequence
gacatccagttgacccagtctccatcctccctgactgcatctgtcggagacagagtcaccattacttgccggtcaagtcagggcattcgcagttctttagcctggtatcaacagaaaccagggaaacctcctaagctcctgatctatggtgcctccagtttggaagtgggggtcccatcaaggttcagcggcagtggatctgggacagatttcactctcaccatcagtagtctgcagcctgaagattttgcaacttattactgtcagcagctcaatagtttcccgtccacttttggccaggggaccaagctggaaatcaaa

SEQ ID NO: 33 - protein, 107 aa, Artificial Sequence
  Antibody sequence
DIQLTQSPSSLTASVGDRVTITCRSSQGIRSSLAWYQQKPGKPPKLLIYGASSLEVGVPSRFSGSGSGTDFTLTISSLQPEDFATYYCQQLNSFPSTFGQGTKLEIK

SEQ ID NO: 34 - DNA, 381 bp, Artificial Sequence
  Antibody sequence
gaggtgcagctgttggagtctgggggaggcttggtacggcctggagggtccctgagactctcctgtgcagcctctggattcaccgcgggtagttatgaaatgaattgggtccgccaggctcccgggaaggggctggagtgggtctcatatataagtagctctggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtgacaatggggcctacggtatggacgtctggggccaagggacaatggtcaccgtctcatca

SEQ ID NO: 35 - protein, 127 aa, Artificial Sequence
  Antibody sequence
EVQLLESGGGLVRPGGSLRLSCAASGFTAGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSS

SEQ ID NO: 36 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtcctcggggggcctacggtatggacgtctggggccaagggacaatggtcaccgtctcatca

SEQ ID NO: 37 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGPRGAYGMDVWGQGTMVTVSS

SEQ ID NO: 38 - DNA, 321 bp, Artificial Sequence
  Antibody sequence
gacatccagttgacccagtctccatcctccctgactgcatctgtcggagacagagtcaccattacttgccggtcaagtcagggcattcgcagttctttagcctggtatcaacagaaaccagggaaacctcctaagctcctgatctatggtgggtccagtttggaaggtggggtcccatcaaggttcagcggcagtggatctgggacagatttcactctcaccatcagtagtctgcagcctgaagattttgcaacttattactgtcagcagctcaatagtttcccgtccacttttggccaggggaccaagctggaaatcaaa

SEQ ID NO: 39 - protein, 107 aa, Artificial Sequence
  Antibody sequence
DIQLTQSPSSLTASVGDRVTITCRSSQGIRSSLAWYQQKPGKPPKLLIYGGSSLEGGVPSRFSGSGSGTDFTLTISSLQPEDFATYYCQQLNSFPSTFGQGTKLEIK

SEQ ID NO: 40 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatggattgggtccgccaggctcccgggaaggggctggagtgggtctcatatataagtagcagtggtgaaacgaccgactacgcagactctgtgaagccgcgcttcaccgtctccagagacaacagtaagaatttactgtatctgcaaatgaacagcctgagagccgaggacacggctgtatattactgtgcgagagacgtgtttttttttgatagtagtggtgacaatggggcctacggtatggacgtctggggccaagggacaatggtcaccgtctcatca

SEQ ID NO: 41 - protein, 127 aa, Artificial Sequence
  Antibody sequence
EVQLLESGGGLVRPGGSLRLSCAASGFTFGSYEMDWVRQAPGKGLEWVSYISSSGETTDYADSVKPRFTVSRDNSKNLLYLQMNSLRAEDTAVYYCARDVFFFDSSGDNGAYGMDVWGQGTMVTVSS

SEQ ID NO: 42 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggttctccgggggcctacggtatggacgtctggggccaagggacaatggtcaccgtctcatca

SEQ ID NO: 43 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGSPGAYGMDVWGQGTMVTVSS

SEQ ID NO: 44 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtgacaatcatgcgtacggtatggacgtctggggccaagggacaatggtcaccgtctcatca

SEQ ID NO: 45 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNHAYGMDVWGQGTMVTVSS

SEQ ID NO: 46 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtcctcctggggcctacggtatggacgtctggggccaagggacaatggtcaccgtctcatca

SEQ ID NO: 47 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGPPGAYGMDVWGQGTMVTVSS

SEQ ID NO: 48 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtcgtccgaatggggcctacggtatggacgtctggggccaagggacaatggtcaccgtctcatca

SEQ ID NO: 49 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSRPNGAYGMDVWGQGTMVTVSS

SEQ ID NO: 50 - DNA, 381 bp, Artificial Sequence
  Antibody sequence
gaggtgcagctgttggagtctgggggaggcttggtacggcctggagggtccctgagactctcctgtgcagcctctggattcaccttcggtagttatgaaatgaattgggtccgccaggctcccgggaaggggctggagtgggtctcatatataagtagcagtggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggttttcctggggcctacggtatggacgtctggggccaagggacaatggtcaccgtctcatca

SEQ ID NO: 51 - protein, 127 aa, Artificial Sequence
  Antibody sequence
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGFPGAYGMDVWGQGTMVTVSS

SEQ ID NO: 52 - DNA, 321 bp, Artificial Sequence
  Antibody sequence
gacatccagttgacccagtctccatcctccctgactgcatctgtcggagacagagtcaccattacttgccggtcaagtcagggcattcgcagttctttagcctggtatcaacagaaaccagggaaacctcctaagctcctgatctatggtgcctccagtttgttgggtggggtcccatcaaggttcagcggcagtggatctgggacagatttcactctcaccatcagtagtctgcagcctgaagattttgcaacttattactgtcagcagctcaatagtttcccgtccacttttggccaggggaccaagctggaaatcaaa

SEQ ID NO: 53 - protein, 107 aa, Artificial Sequence
  Antibody sequence
DIQLTQSPSSLTASVGDRVTITCRSSQGIRSSLAWYQQKPGKPPKLLIYGASSLLGGVPSRFSGSGSGTDFTLTISSLQPEDFATYYCQQLNSFPSTFGQGTKLEIK

SEQ ID NO: 54 - DNA, 381 bp, Artificial Sequence
  Antibody sequence
gaggtgcagctgttggagtctgggggaggcttggtacggcccggagggtccctgagactctcctgtgcagcctctggattcacccctggtagttatgaaatgaattgggtccgccaggctcccgggaaggggctggagtgggtctcatatataagtagcgcgggtgaaacgaccgactacgcagactctgtgaagggccgcttcaccgtctccagagacaacagtaagaatttactgtatctgcaaatgaacagcctgagagccgaggacacggctgtatattactgtgcgagagacgtgtttttttttgatagtagtggtgacaatggggcctacggtatggacgtctggggccaagggacaatggtcaccgtctcatca

SEQ ID NO: 55 - protein, 127 aa, Artificial Sequence
  Antibody sequence
EVQLLESGGGLVRPGGSLRLSCAASGFTPGSYEMNWVRQAPGKGLEWVSYISSAGETTDYADSVKGRFTVSRDNSKNLLYLQMNSLRAEDTAVYYCARDVFFFDSSGDNGAYGMDVWGQGTMVTVSS

SEQ ID NO: 56 - protein, 7 aa, Artificial Sequence
  Antibody sequence
  MISC_FEATURE (2)..(2): Xaa may be Ala or Gly
  MISC_FEATURE (6)..(6): Xaa may be Glu or Leu
  MISC_FEATURE (7)..(7): Xaa may be Gly or Val
GXSSLXX

SEQ ID NO: 57 - protein, 9 aa, Artificial Sequence
  Antibody sequence
  MISC_FEATURE (4)..(4): Xaa may be Asn or Ala
QQLXSFPST

SEQ ID NO: 58 - protein, 10 aa, Artificial Sequence
  Antibody sequence
  MISC_FEATURE (4)..(4): Xaa may be Phe, Ala or Pro
  MISC_FEATURE (10)..(10): Xaa may be Asn or Asp
GFTXGSYEMX

SEQ ID NO: 59 - protein, 10 aa, Artificial Sequence
  Antibody sequence
  MISC_FEATURE (5)..(5): Xaa may be Ser or Ala
YISSXGETTD

SEQ ID NO: 60 - protein, 18 aa, Artificial Sequence
  Antibody sequence
  MISC_FEATURE (6)..(6): Xaa may be Asp or Ser
  MISC_FEATURE (7)..(7): Xaa may be Ser or Pro
  MISC_FEATURE (9)..(9): Xaa may be Gly or Arg
  MISC_FEATURE (10)..(10): Xaa may be Asp, Pro, Ser or Phe
  MISC_FEATURE (11)..(11): Xaa may be Pro, Asn or Arg
  MISC_FEATURE (12)..(12): Xaa may be Gly or His
DVFFFXXSXXXXAYGMDV

SEQ ID NO: 61 - protein, 214 aa, Artificial Sequence
  Antibody sequence
DIQMTQSPSSLSASVGDRVTITCRASQDISNYLNWYQQKPGKAPKLLIYYTSRLHSGVPSRFSGSGSGTDYTLTISNLQPEDIATYYCQQGKNYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 452 aa, Artificial Sequence
  Antibody sequence
QVQLVQSGAEVKKPGASVKMSCKASGYTFTSSVMHWVRQAPGQGLEWIGYINPYTDGTKYAQKFQGRATLTSDKSTSTAYMEFSSLRSEDTAVYYCGRPYYGTRYGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0218071 A1
(43) Pub. Date: Jul. 4, 2024

(54) ANTI-CD40 ANTIBODY AND USE THEREOF

(71) Applicants: ANHUI BIOX-VISION BIOLOGICAL TECHNOLOGY CO., LTD., Anhui (CN); SANYOU BIOPHARMACEUTICALS CO., LTD., Shanghai (CN)

(72) Inventors: Guojun LANG, Shanghai (CN); Guoxing WANG, Shanghai (CN); Wenhai ZHANG, Shanghai (CN); Run YAN, Shanghai (CN); Peipei LIU, Shanghai (CN)

(73) Assignees: ANHUI BIOX-VISION BIOLOGICAL TECHNOLOGY CO., LTD., Anhui (CN); SANYOU BIOPHARMACEUTICALS CO., LTD., Shanghai (CN)

(21) Appl. No.: 18/558,534

(22) PCT Filed: May 26, 2022
(86) PCT No.: PCT/CN2022/095229

§ 371 Date: Nov. 2, 2023

(30) Foreign Application Priority Data
May 26, 2021 (CN) 202110577432.9

(51) Int. Cl. C07K 16/28; A61K 39/00; A61K 45/06; A61K 47/68; A61P 35/00; G01N 33/68

(52) U.S. Cl. CPC C07K 16/2878; A61K 45/06; A61K 47/6849; A61P 35/00; G01N 33/6854; A61K 2039/505; C07K 2317/524; C07K 2317/565; C07K 2317/75; C07K 2317/92

(57) ABSTRACT

The present invention relates to an anti-CD40 antibody or an antigen-binding fragment thereof that specifically recognizes human, cynomolgus monkey and mouse CD40 molecules and weakly agonizes the CD40 molecules, and an immunoconjugate, a pharmaceutical composition and a combined product containing the anti-CD40 antibody or the antigen-binding fragment thereof. The present invention also relates to a nucleic acid encoding the anti-CD40 antibody or the antigen-binding fragment thereof, a host cell containing the nucleic acid, and a method for preparing the anti-CD40 antibody or the antigen-binding fragment thereof. The present invention further relates to the use of the anti-CD40 antibody or the antigen-binding fragment thereof in the prevention or treatment of tumors or infectious diseases in a subject.

### BACKGROUND OF THE INVENTION

### 1. Technical Field

The present invention relates to an anti-CD40 antibody or an antigen-binding fragment thereof. Particularly, the present invention relates to an anti-CD40 antibody or an antigen-binding fragment thereof that specifically recognizes human, cynomolgus monkey and mouse CD40 molecules and weakly agonizes the CD40 molecules, and an immunoconjugate, a pharmaceutical composition and a combined product containing the anti-CD40 antibody or the antigen-binding fragment thereof. The present invention also relates to a nucleic acid encoding the anti-CD40 antibody or the antigen-binding fragment thereof, a host cell containing the nucleic acid, and a method for preparing the anti-CD40 antibody or the antigen-binding fragment thereof. The present invention further relates to the use of the anti-CD40 antibody or the antigen-binding fragment thereof in the prevention or treatment of tumors or infectious diseases in a subject.

### 2. Background Art

Clinical trials of CD40 agonists have shown that they (such as selicrelumab, dacetuzumab and APX005M) exhibit clinical activities in a variety of indications (Hassan S B et al., Anti-CD40-mediated cancer immunotherapy: an update of recent and ongoing clinical trials. Immunopharmacol Immunotoxicol 2014; 36: 96-104). However, these studies have also shown that the CD40 agonists are associated with adverse events leading to dose-limiting toxicity. The most common adverse events include cytokine release syndrome (CRS) and hepatotoxicity. At present, CD40 agonists having normal Fc function are commonly used in clinical trials, and such agonists can be divided into two categories according to the agonistic activity. Some of these agonists are highly toxic due to their ability to nonspecifically over-activate the immune response and are not suitable as therapeutic agents. While others need to be subjected to a crosslinking effect to form anti-CD40 monoclonal antibodies having immune activity, resulting in a local immunopotentiating effect by a limited controllable activation of CD40-expressing immune cells, thereby killing tumor cells in a limited range.

In recent years, therapeutic index (TI) has increasingly become an important index for evaluating drug safety. TI is a comparison between the dosage of a drug that causes a therapeutic effect and the dosage that causes toxicity, which means that at a drug dose administered to a patient, if the efficacy of the drug is higher and the toxicity is lower, the therapeutic index is higher and the safety is higher. However, in the prior art, the currently known anti-CD40 antibodies have a low therapeutic index, and the ratio of the immune cell killing activity formed by the antibody at the lesion site to the non-specific agonistic activity (such as hepatotoxicity) caused at other sites is not high at the dose actually administered to a patient. Therefore, there is a need in the art to develop new CD40 agonists that can both provide sufficient immune stimulation and reduce adverse events caused by traditional CD40 agonists.

In addition, in the development of new CD40 agonists, evaluation with animal models in the preclinical phase is an important link in the evaluation of drug safety and therapeutic efficacy. Rodents (for example, mice) are widely used to establish animal models of human diseases. However, anti-CD40 antibodies that have no cross-reactivity to mouse CD40 (such as selicrelumab developed by Roche) need to be used in a human CD40 transgenic mouse tumor model to reflect their in vivo efficacy and safety. In testing the efficacy of anti-CD40 bispecific antibodies in mouse models, there is also an urgent need to develop an agonistic anti-CD40 antibody having cross-reactivity to mouse CD40.

### SUMMARY OF THE INVENTION

The present invention provides an agonistic anti-CD40 antibody with a high therapeutic index, wherein the antibody is capable of attenuating adverse effects of non-specific immune activation caused by CD40 activation while providing sufficient immune stimulation in a subject, and has one or more of the following properties:
(1) binding to and activating CD40 (such as human CD40, cynomolgus monkey CD40 and mouse CD40) with a high affinity, for example, binding to CD40 with an affinity of about 10⁻⁷M to about 10⁻¹⁰M, as measured by ForteBio kinetic binding assay;
(2) enhancing a binding of CD40 to CD40L;
(3) activating an antigen presenting cell, including, e.g., a dendritic cell (DC), a B cell, a monocyte and a macrophage, by binding to CD40 expressed on the antigen presenting cell;
(4) inducing a CD40-expressing B cell to express CD95;
(5) significantly enhancing a B cell-mediated immune response when a crosslinking effect is formed;
and (6) barely or weakly enhancing a B cell-mediated immune response when a crosslinking effect is not formed.

The anti-CD40 antibody of the present invention has a complete human sequence and is a human antibody. It is expected that the anti-CD40 antibody of the present invention exhibits minimal immunogenicity in human subjects, which induces fewer anti-drug antibodies (ADA), so that the ADA-related clearance of therapeutic anti-CD40 antibodies is minimized, and human subjects will have a good tolerance to the therapeutic anti-CD40 antibodies.

In some embodiments, the anti-CD40 antibody of the present invention comprises a light chain variable region and a heavy chain variable region. The light chain variable region comprising three complementarity determining regions, i.e., LCDR1, LCDR2 and LCDR3, respectively, and the heavy chain variable region comprising three complementarity determining regions, i.e., HCDR1, HCDR2 and HCDR3, respectively, wherein:
(a) LCDR1 comprises an amino acid sequence as shown in SEQ ID NO: 1 or a variant of SEQ ID NO: 1 having no more than 2 or no more than 1 amino acid change;
(b) LCDR2 comprises an amino acid sequence selected from any one of amino acid sequences of SEQ ID NOs: 2, 5, 6 and 7, or a variant of any one of amino acid sequences of SEQ ID NOs: 2, 5, 6 and 7 having no more than 2 or no more than 1 amino acid change;
(c) LCDR3 comprises an amino acid sequence selected from any one of amino acid sequences of SEQ ID NOs: 3 and 4, or a variant of any one of amino acid sequences of SEQ ID NOs: 3 and 4 having no more than 2 or no more than 1 amino acid change;
(d) HCDR1 comprises an amino acid sequence selected from any one of amino acid sequences of SEQ ID NOs: 8, 12, 14 and 20, or a variant of any one of amino acid sequences of SEQ ID NOs: 8, 12, 14 and 20 having no more than 2 or no more than 1 amino acid change;
(e) HCDR2 comprises an amino acid sequence selected from any one of amino acid sequences of SEQ ID NOs: 9 and 21, or a variant of any one of amino acid sequences of SEQ ID NOs: 9 and 21 having no more than 2 or no more than 1 amino acid change;
and (f) HCDR3 comprises an amino acid sequence selected from any one of amino acid sequences of SEQ ID NOs: 10, 11, 13, 15, 16, 17, 18 and 19, or a variant of any one of amino acid sequences of SEQ ID NOs: 10, 11, 13, 15, 16, 17, 18 and 19 having no more than 2 or no more than 1 amino acid change,
wherein the amino acid change is an amino acid addition, deletion or substitution.

In some embodiments, the anti-CD40 antibody of the present invention comprises
(i) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 10 or SEQ ID NO: 10 having no more than 2 or no more than 1 amino acid change;
(ii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 4 or SEQ ID NO: 4 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 10 or SEQ ID NO: 10 having no more than 2 or no more than 1 amino acid change;
(iii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 11 or SEQ ID NO: 11 having no more than 2 or no more than 1 amino acid change;
(iv) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 5 or SEQ ID NO: 5 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 12 or SEQ ID NO: 12 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 10 or SEQ ID NO: 10 having no more than 2 or no more than 1 amino acid change;
(v) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 13 or SEQ ID NO: 13 having no more than 2 or no more than 1 amino acid change;
(vi) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 6 or SEQ ID NO: 6 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 14 or SEQ ID NO: 14 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 10 or SEQ ID NO: 10 having no more than 2 or no more than 1 amino acid change;
(vii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 15 or SEQ ID NO: 15 having no more than 2 or no more than 1 amino acid change;
(viii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 16 or SEQ ID NO: 16 having no more than 2 or no more than 1 amino acid change;
(ix) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 17 or SEQ ID NO: 17 having no more than 2 or no more than 1 amino acid change;
(x) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 18 or SEQ ID NO: 18 having no more than 2 or no more than 1 amino acid change;
(xi) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and LCDR3 comprising an amino acid sequence of SEQ ID NO: 19 or SEQ ID NO: 19 having no more than 2 or no more than 1 amino acid change; or
(xii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 7 or SEQ ID NO: 7 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 20 or SEQ ID NO: 20 having no more than 2 or no more than 1 amino acid change, HCDR2 comprising an amino acid sequence of SEQ ID NO: 21 or SEQ ID NO: 21 having no more than 2 or no more than 1 amino acid change, and LCDR3 comprising an amino acid sequence of SEQ ID NO: 10 or SEQ ID NO: 10 having no more than 2 or no more than 1 amino acid change.

In some embodiments, the anti-CD40 antibody of the present invention comprises:
LCDR1 comprising an amino acid sequence RSSQGIRSSLA (SEQ ID NO: 1), LCDR2 comprising an amino acid sequence GX₁SSLX₂X₃ (SEQ ID NO: 56), and LCDR3 comprising an amino acid sequence QQLX₄SFPST (SEQ ID NO: 57), wherein X₁ is A or G, X₂ is E or L, X₃ is G or V, and X₄ is N or A; and
HCDR1 comprising an amino acid sequence GFTX₅GSYEMX₆ (SEQ ID NO: 58), HCDR2 comprising an amino acid sequence YISSX₇GETTD (SEQ ID NO: 59), and HCDR3 comprising an amino acid sequence DVFFFX₈X₉SX₁₀X₁₁X₁₂X₁₃AYGMDV (SEQ ID NO: 60), wherein X₅ is F, A or P, X₆ is N or D, X₇ is S or A, X₈ is D or S, X₉ is S or P, X₁₀ is G or R, X₁₁ is D, P, S or F, X₁₂ is P, Nor R, and X₁₃ is G or H.

In some specific embodiments, the anti-CD40 antibody of the present invention comprises:
(i) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 10;
(ii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 4, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 10;
(iii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 11;
(iv) LCDR1 comprising an amino acid sequence of SEQ ID NO: 1, LCDR2 comprising an amino acid sequence of SEQ ID NO: 5, LCDR3 comprising an amino acid sequence of SEQ ID NO: 3, HCDR1 comprising an amino acid sequence of SEQ ID NO: 12, HCDR2 comprising an amino acid sequence of SEQ ID NO: 9, and LCDR3 comprising an amino acid sequence of SEQ ID NO: 10;
(v) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 13;
(vi) LCDR1 comprising an amino acid sequence of SEQ ID NO: 1, LCDR2 comprising an amino acid sequence of SEQ ID NO: 6, LCDR3 comprising an amino acid sequence of SEQ ID NO: 3, HCDR1 comprising an amino acid sequence of SEQ ID NO: 14, HCDR2 comprising an amino acid sequence of SEQ ID NO: 9, and LCDR3 comprising an amino acid sequence of SEQ ID NO: 10;
(vii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 15;
(viii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 16;
(ix) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 17;
(x) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 18;
(xi) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and LCDR3 comprising an amino acid sequence of SEQ ID NO: 19; or
(xii) LCDR1 comprising an amino acid sequence of SEQ ID NO: 1, LCDR2 comprising an amino acid sequence of SEQ ID NO: 7, LCDR3 comprising an amino acid sequence of SEQ ID NO: 3, HCDR1 comprising an amino acid sequence of SEQ ID NO: 20, HCDR2 comprising an amino acid sequence of SEQ ID NO: 21, and LCDR3 comprising an amino acid sequence of SEQ ID NO: 10.

In some embodiments, the anti-CD40 antibody of the present invention comprises a light chain variable region and a heavy chain variable region, wherein:
(i) the light chain variable region comprises any one of amino acid sequences of SEQ ID NOs: 23, 29, 33, 39 and 53, and the heavy chain variable region comprises any one of amino acid sequences of SEQ ID NOs: 25, 31, 35, 37, 41, 43, 45, 47, 49, 51 and 55; or
(ii) the light chain variable region comprises an amino acid sequence having at least 85%, 90%, 91%, 92%, 93%, 94%, 95%, 96%, 97%, 98% or 99% identity to any one of amino acid sequences of SEQ ID NOs: 23, 29, 33, 39 and 53, and the heavy chain variable region comprises an amino acid sequence having at least 85%, 90%, 91%, 92%, 93%, 94%, 95%, 96%, 97%, 98% or 99% identity to any one of amino acid sequences of SEQ ID NOs: 25, 31, 35, 37, 41, 43, 45, 47, 49, 51 and 55.

In some embodiments, the anti-CD40 antibody of the present invention comprises:
(i) a light chain variable region comprising an amino acid sequence of SEQ ID NO: 23 and a heavy chain variable region comprising an amino acid sequence of SEQ ID NO: 25;
(ii) a light chain variable region comprising an amino acid sequence of SEQ ID NO: 29 and a heavy chain variable region comprising an amino acid sequence of SEQ ID NO: 25;
(iii) a light chain variable region comprising an amino acid sequence of SEQ ID NO: 23 and a heavy chain variable region comprising an amino acid sequence of SEQ ID NO: 31;
(iv) a light chain variable region comprising an amino acid sequence of SEQ ID NO: 33 and a heavy chain variable region comprising an amino acid sequence of SEQ ID NO: 35;
(v) a light chain variable region comprising an amino acid sequence of SEQ ID NO: 23 and a heavy chain variable region comprising an amino acid sequence of SEQ ID NO: 37;
(vi) a light chain variable region comprising an amino acid sequence of SEQ ID NO: 39 and a heavy chain variable region comprising an amino acid sequence of SEQ ID NO: 41;
(vii) a light chain variable region comprising an amino acid sequence of SEQ ID NO: 23 and a heavy chain variable region comprising an amino acid sequence of SEQ ID NO: 43;
(viii) a light chain variable region comprising an amino acid sequence of SEQ ID NO: 23 and a heavy chain variable region comprising an amino acid sequence of SEQ ID NO: 45;
(ix) a light chain variable region comprising an amino acid sequence of SEQ ID NO: 23 and a heavy chain variable region comprising an amino acid sequence of SEQ ID NO: 47;
(x) a light chain variable region comprising an amino acid sequence of SEQ ID NO: 23 and a heavy chain variable region comprising an amino acid sequence of SEQ ID NO: 49;
(xi) a light chain variable region comprising an amino acid sequence of SEQ ID NO: 23 and a heavy chain variable region comprising an amino acid sequence of SEQ ID NO: 51; or
(xii) a light chain variable region comprising an amino acid sequence of SEQ ID NO: 53 and a heavy chain variable region comprising an amino acid sequence of SEQ ID NO: 55, preferably, the anti-CD40 antibody or the antigen-binding fragment is a fully human antibody.

In some embodiments, the anti-CD40 antibody of the present invention is an IgG1, IgG2, IgG3, or IgG4 antibody; preferably an IgG1 or IgG4 antibody; and more preferably a human IgG1 or IgG4 antibody.

In some embodiments, the anti-CD40 antibody of the present invention is an antigen-binding fragment, including but not limited to Fab, Fab′, F(ab′)2, Fv, single chain Fv, single chain Fab and diabody.

In some embodiments, a glycosylation site in a CH2 domain of an immunoglobulin Fc region of the anti-CD40 antibody of the present invention is removed, for example, the glycosylation site is removed by mutating N297 residue in a CH2 domain of a human IgG Fc region, e.g., mutating N297 residue to Gly, Ala, Gln, Asp or Glu, preferably mutating N297 residue to Gln.

In a second aspect, the present invention provides a method for preparing the anti-CD40 antibody of the present invention, wherein the method comprises culturing a host cell, into which a nucleic acid encoding the anti-CD40 antibody of the present invention or an expression vector containing the nucleic acid is introduced, under conditions suitable for the expression of the nucleic acid encoding the anti-CD40 antibody of the present invention, and isolating the anti-CD40 antibody, and optionally the method further comprising: recovering the anti-CD40 antibody from the host cell.

In a third aspect, the present invention provides an immunoconjugate, comprising the anti-CD40 antibody of the present invention and other substances such as a cytotoxic agent.

In a fourth aspect, the present invention provides a pharmaceutical composition, comprising the anti-CD40 antibody or immunoconjugate of the present invention, and optionally a pharmaceutical adjuvant material.

In some embodiments, the present invention provides a pharmaceutical composition, comprising the anti-CD40 antibody or immunoconjugate of the present invention, and other therapeutic agents, and optionally a pharmaceutical adjuvant material; preferably, the other therapeutic agents are selected from a chemotherapeutic agent, other antibodies (such as an anti-PD-1 antibody or an anti-PD-L1 antibody) and a cytotoxic agent.

In some embodiments, the present invention provides a combined product, comprising the anti-CD40 antibody or immunoconjugate of the present invention, and one or more other therapeutic agents, such as a chemotherapeutic agent, a cytotoxic agent and other antibodies, preferably an anti-PD-1 antibody or an anti-PD-L1 antibody.

In a fifth aspect, the present invention provides a method for preventing or treating a tumor or an infectious disease in a subject or an individual, wherein the method comprises administering to the subject an effective amount of the anti-CD40 antibody, immunoconjugate, pharmaceutical composition or combined product of the present invention.

In some embodiments, the tumor to be prevented or treated with the anti-CD40 antibody, immunoconjugate, pharmaceutical composition or combined product of the present invention is a cancer, for example, a cancer expressing a CD40 molecule or a cancer not expressing a CD40 molecule; or the infectious disease to be prevented or treated with the anti-CD40 antibody, immunoconjugate, pharmaceutical composition or combined product of the present invention is, for example, a bacterial infection, a viral infection, a fungal infection or a protozoal infection, preferably the infectious disease is a chronic infection in which a subject or an individual suffers from hypoimmunity.

In a sixth aspect, the present invention provides a kit for detecting CD40 in a sample, wherein the kit comprises the anti-CD40 antibody of the present invention and is used for performing the following steps:
(a) contacting the sample with the anti-CD40 antibody of the present invention; and
(b) detecting the formation of a complex of the anti-CD40 antibody and CD40; optionally, the anti-CD40 antibody is detectably labeled, and therefore, the expression level of CD40 in the sample from a subject or an individual is determined.

### BRIEF DESCRIPTION OF THE DRAWINGS

The following detailed description of the preferred embodiments of the present invention can be better understood when reading in conjunction with the following drawings below. For the purpose of illustrating the present invention, there are shown in the drawings embodiments which are presently preferred. However, it should be understood that the present invention is not limited to the precise arrangements and instrumentalities of the embodiments shown in the drawings.

FIG. 1 shows the binding of CD40 antigen P17-Fc to two control antibodies, dacetuzumab and bleslumab, respectively. A human IgG1 isotype antibody was used as a negative control. The abscissa in the figure is the antibody concentration.

FIG. 2 shows the comparison of the binding of unbiotinylated P17-Fc and biotinylated P17-Fc to two control antibodies, dacetuzumab and bleslumab, respectively. In the figure, “P17-Fc-CD40-ble” represents “binding of unbiotinylated P17-Fc to bleslumab (also referred to herein as CD40-Ble or CD40-ble)”; and “P17-Fc-CD40-dac” represents “binding of unbiotinylated P17-Fc to dacetuzumab (also referred to herein as CD40-Dac or CD40-dac)”.

FIG. 3 shows the ELISA result of a phage library after three rounds of panning on a human natural antibody phage display library. In the figure, “VCSM13” represents “VCSM13 helper phage”, obtained from Stratagene (USA).

FIG. 4 shows the binding affinity of C8 clone CD40 Fab molecule (screened from a human natural antibody phage display library) for CD40, with bleslumab as a positive control and a human IgG1 isotype antibody as a negative control. The abscissa in the figure is the antibody concentration.

FIG. 5A shows the binding of a C8 clone anti-CD40 Fab molecule to human CD40 stably expressed on CHO cells.

FIG. 5B shows the cross-reactivity of a C8 clone anti-CD40 Fab molecule to cynomolgus monkey CD40 stably expressed on CHO cells.

FIG. 6A shows the affinity of a full-length anti-CD40 candidate antibody for human CD40 stably expressed on CHO cells.

FIG. 6B shows the affinity of a full-length anti-CD40 candidate antibody for cynomolgus monkey CD40 stably expressed on CHO cells.

FIG. 6C shows the affinity of a full-length anti-CD40 candidate antibody for mouse CD40 stably expressed on CHO cells. A human IgG1 isotype antibody was used as a negative control.

FIG. 7 shows the effect of a full-length anti-CD40 antibody on the binding of CD40L to CHO cells expressing CD40.

FIG. 8A and FIG. 8B show the anti-CD40 antibody-induced iDC activation assay, which is demonstrated by IL-12 secretion determined by ELISA and CD83 expression determined by FACS.

FIG. 9 shows the anti-CD40 antibody-induced CD95 expression on Ramos cells as measured by FACS.

FIG. 10A shows the final tumor volume of Ramos cell mouse tumor models after administration with an anti-CD40 antibody.

FIG. 10B shows the final tumor weight of Ramos cell mouse tumor models after administration with an anti-CD40 antibody.

FIG. 10C shows the change in the tumor volume of Ramos cell mouse tumor models over time after administration with an anti-CD40 antibody. NS in the figure indicates that there is no significant difference; * indicates that p<0.05 compared to PBS group; ** indicates that p<0.01; and *** indicates that p<0.001.

FIG. 10D shows the final tumor weight after administration with an anti-CD40 antibody in MC-38 cell tumor models.

FIG. 11A shows the non-specific binding of affinity variant full-length candidate antibodies on CHO-K cells.

FIG. 11B shows the non-specific binding of affinity variant full-length candidate antibodies on HEK293 cells.

FIG. 12A and FIG. 12B show the affinity of affinity variant full-length candidate antibodies for human CD40 stably expressed on CHO cells.

FIG. 12C and FIG. 12D show the affinity of affinity matured full-length candidate antibodies for mouse CD40 stably expressed on CHO cells.

FIG. 13A and FIG. 13B show the CD95 expression on Ramos cells induced by C8 molecules and affinity variant full-length candidate antibodies thereof in the presence of a crosslinking agent as measured by FACS.

FIG. 13C and FIG. 13D show the CD95 expression on Ramos cells induced by C8 molecules and affinity variant full-length candidate antibodies thereof in the absence of a crosslinking agent as measured by FACS.

FIG. 14A-FIG. 14D show the ability of C8 molecules and affinity variant full-length candidate antibodies thereof to induce NF-κB luciferase reporter gene expression in the presence of a crosslinking agent.

FIG. 15A-FIG. 15D show the ability of C8 molecules and affinity variant full-length candidate antibodies thereof to induce NF-κB luciferase reporter gene expression in the absence of a crosslinking agent.

### DETAILED DESCRIPTION OF THE EMBODIMENTS

Unless defined otherwise, all technical and scientific terms used herein have the same meanings as commonly understood by those of ordinary skill in the art to which the present invention pertains. All publications, patent applications, patents and other references mentioned herein are incorporated by reference in its entirety. In addition, the materials, methods and examples described herein are illustrative only and are not intended to be limiting. Other features, objectives and advantages of the present invention will be apparent from the description, the drawings and the appended claims.

### I. Definitions

For interpreting this specification, the following definitions will apply and whenever appropriate, terms used in the singular will also include the plural and vice versa. It is to be understood that the terminology used herein is for the purpose of describing particular embodiments only, and is not intended to be limiting.

The term “about” when used in conjunction with a numerical value is meant to encompass numerical values within a range having a lower limit that is 5% smaller than the specified numerical value and an upper limit that is 5% larger than the specified numerical value.

As used herein, the term “and/or” means any one of optional items or two or more of the optional items.

Unless otherwise indicated, the term “comprise”, “contain” or “include” when used herein also encompasses situations consisting of the described elements, integers or steps. For example, when referring to an antibody variable region “containing” a particular sequence, it is intended to also encompass an antibody variable region consisting of that particular sequence.

The term “CD40-expressing cell” refers to any cell that expresses a CD40 molecule, including but not limited to, an antigen presenting cell (APC), including a dendritic cell (DC), a B cell, a macrophage and a monocyte. CD40 is also expressed on other types of cells, such as epithelial cells, endothelial cells and platelets. It has also been confirmed that CD40 is expressed on a variety of tumor cells, including B-cell lymphoma and kidney cancer cells. In a specific embodiment, the CD40-expressing cell includes a CD40-expressing cell line, such as Jurkat cell, Raji cell, Ramos cell and Daudi cell. In another embodiment, the CD40-expressing cell is a tumor cell or a cancer cell. In another embodiment, the CD40-expressing cell includes a B cell, a NK cell and a T cell that are found to infiltrate tumors, also referred to as tumor infiltrating lymphocytes.

The term “antigen presenting cell” or “APC” is a cell that displays a foreign antigen complex with MHC on its surface. T cell recognizes this complex using a T cell receptor (TCR). Examples of APCs include, but are not limited to, dendritic cells (DCs), peripheral blood mononuclear cells (PBMCs), monocytes, B lymphoblastoid cells and monocyte-derived dendritic cells (DCs).

The term “antigen presenting” refers to a process by which an APC captures an antigen and enables the antigen to be recognized by a T cell, for example, an antigen is processed into a component of an MHC-I and/or MHC-II conjugates.

The “MHC molecule” includes two types of molecules, MHC class I and MHC class II molecules. MHC class I molecules present antigens to specific CD8+ T cells, and MHC class II molecules present antigens to specific CD4+ T cells. Antigens exogenously delivered to APCs are primarily used to bind to MHC class II molecules. Conversely, antigens endogenously delivered to APCs are primarily used to bind to MHC class I molecules.

The term “T cell-mediated response” refers to any response mediated by T cells (including effector T cells (such as CD8+ cells) and helper T cells (such as CD4+ cells)). T cell-mediated responses include, for example, cytotoxicity and proliferation of T cells.

The term “antibody” herein is used in the broadest sense, refers to a protein comprising an antigen-binding site, and encompasses natural antibodies and artificial antibodies of various structures, including but not limited to monoclonal antibodies, polyclonal antibodies, multispecific antibodies (such as bispecific antibodies), single chain antibodies, intact antibodies, and antibody fragments.

The “antibody fragment” or “antigen-binding fragment” refers to a molecule different from an intact antibody, which contains a portion of the intact antibody and binds to an antigen to which the intact antibody binds. Examples of antibody fragments include, but are not limited to, Fv, Fab, Fab′, Fab′-SH, F(ab′)2, diabodies, linear antibodies, single chain antibodies (such as scFv), single domain antibodies, bivalent or bispecific antibodies or fragments thereof, camelid antibodies (heavy chain antibodies), and bispecific or multispecific antibodies formed from antibody fragments.

The term “cross-reactivity” refers to the ability of the antibody of the present invention to bind to CD40 from different species. For example, the antibody of the present invention that binds to human CD40 may also bind to CD40 of another species. Cross-reactivity is measured by detecting the specific reactivity with a purified CD40 antigen or the binding to or otherwise functionally interacting with cells physiologically expressing CD40 in a binding assay (such as SPR and ELISA). Methods for determining cross-reactivity include, for example, biolayer interferometry (BLI) using an Octet™ QKe instrument or Biacore™ surface plasmon resonance (SPR) analysis using Biacore™ 2000 SPR instrument (Biacore AB, Uppsala, Sweden) or flow cytometry techniques.

The “complementarity determining region” or “CDR region” or “CDR” is a region of an antibody variable domain which is hypervariable in the sequence and forms a structurally defined loop (“hypervariable loop”) and/or contains an antigen contact residue (“antigen contact point”). CDR is primarily responsible for binding to an epitope of an antigen. CDRs of a heavy chain are generally referred to as CDR1, CDR2 and CDR3, numbered sequentially starting from N-terminus. In a given heavy chain variable region amino acid sequence, the precise amino acid sequence boundary of each CDR may be determined by using any one of many well-known antibody CDR assignment systems or a combination thereof, wherein the assignment system includes, for example, Chothia based on the three-dimensional structures of antibodies and the topology of CDR loops (Chothia et al., (1989) Nature 342: 877-883, Al-Lazikani et al., “Standard conformations for the canonical structures of immunoglobulins”, Journal of Molecular Biology, 273, 927-948 (1997)), Kabat based on antibody sequence variability (Kabat et al., Sequences of Proteins of Immunological Interest, 4th edition, U.S. Department of Health and Human Services, National Institutes of Health (1987)), AbM (University of Bath), Contact (University College London), International ImMunoGeneTics database (IMGT) (http://imgt.cines.fr/), and the North CDR definition based on affinity propagation clustering using a large number of crystal structures.

Unless otherwise stated, in the present invention, the term “CDR” or “CDR sequence” encompass CDR sequences determined by any one of the above methods.

CDR can also be determined on the basis of having the same AbM numbering position as a reference CDR sequence (such as any one of the exemplary CDRs of the present invention). In one embodiment, the position of the CDR of the antibody of the present invention is determined according to an AbM numbering scheme.

Unless otherwise stated, in the present invention, when referring to residue positions (including heavy chain variable region residues) in antibody variable regions and CDRs, it is referred to as numbering positions according to an AbM numbering system.

Antibodies having different specificity (i.e., different binding sites for different antigens) have different CDRs. However, although CDRs vary from antibody to antibody, only a limited number of amino acid positions within the CDRs are directly involved in antigen binding. Using at least two of the Kabat, Chothia, AbM and Contact methods, a minimal overlap region can be determined, thereby providing a “minimal binding unit” for antigen binding. The minimal binding unit may be a sub-portion of a CDR. As will be apparent to a person skilled in the art, the residues of the remainder of the CDR sequence can be determined by the structure of an antibody and by protein folding. Therefore, the present invention also takes variants of any of the CDRs given herein. For example, in a variant of a CDR, the amino acid residue of the minimal binding unit can remain unchanged, while the remaining CDR residues defined according to Kabat or Chothia or AbM can be replaced with conserved amino acid residues.

The “human antibody” refers to an antibody having an amino acid sequence corresponding to that of an antibody produced by human or human cells or derived from a non-human source using human antibody repertoire or other human antibody coding sequences. This definition of a human antibody explicitly excludes a humanized antibody comprising non-human antigen-binding residues.

The term “Fc region” herein is used to define a C-terminal region of an immunoglobulin heavy chain, comprising at least a portion of the constant region. The term includes a natural sequence Fc region and a variant Fc region. In certain embodiments, a human IgG heavy chain Fc region can extend from Cys226 or Pro230 to the carboxyl terminus of the heavy chain. However, the C-terminal lysine (Lys447) of an Fc region may or may not be present. Unless otherwise stated, the amino acid residues in an Fc region or a constant region is numbered according to the EU numbering system, also known as the EU index, as described in Kabat et al., Sequences of Proteins of Immunological Interest, 5th Ed. Public Health Service, National Institutes of Health, Bethesda, M D, 1991.

The term “variable region” or “variable domain” refers to an antibody heavy or light chain domain that is involved in the binding of an antibody to an antigen. The heavy and light chain variable domains of a natural antibody generally have similar structures, with each domain comprising four conserved framework regions (FRs) and three complementarity determining regions (CDRs) (See, for example, Kindt et al., Kuby Immunology, 6^{th} ed., W. H. Freeman and Co., page 91 (2007)). A single VH or VL domain may be sufficient to confer antigen-binding specificity.

As used herein, the term “binding” or “specific binding” means that the binding is selective for an antigen and can be distinguished from unwanted or non-specific interactions. The ability of an antibody to bind to a particular antigen can be determined by enzyme-linked immunosorbent assay (ELISA), SPR or biolayer interferometry or other conventional binding assays known in the art.

The term “costimulatory molecule” refers to cell surface molecules and ligands thereof that provide a costimulatory signal for the full activation of T cells or B cells. Costimulatory molecules are cell surface molecules other than antigen receptors or ligands thereof that contribute to an effective immune response. Costimulatory molecules include, but are not limited to, MHC class I molecules, TNF receptor proteins, immunoglobulin-like proteins, cytokine receptors, integrins, signaling lymphocyte activating molecules (SLAM proteins), activated NK cell receptors, OX40, CD40, GITR, 4-1BB (i.e., CD137), CD27, CD28, etc.

The term “cytokine” is a generic term for proteins released by a cell population to act as an intercellular mediator on another cell. Examples of such cytokines are lymphokines, monokines, interleukins (ILs), such as IL-1, IL-la, IL-2, IL-3, IL-4, IL-5, IL-6, IL-7, IL-8, IL-9, IL-11, IL-12 and IL-15; tumor necrosis factors such as TNF-α or TNF-β; and other polypeptide factors including γ-interferon.

The “immunoconjugate” refers to an antibody conjugated to one or more other substances (including but not limited to a cytotoxic agent or a label).

The terms “induce” and “increase” used in inducing CDC or ADCC refer to stimulating particular direct cell killing mechanisms. For example, in one embodiment, ADCC caused by the anti-CD40 antibody of the present invention through the activation of a CD40-expressing cell results in the lysis of at least about 20%, 25%, 30%, 35%, 40%, 45%, 50%, 55%, 60%, 65%, 70%, 75%, 80%, 85%, 90%, 95% of tumor cells or cells infected with viruses, bacteria, etc.

The term “agonize” refers to increasing certain parameters (such as activity) of a given molecule (such as a costimulatory molecule). For example, this term includes substances that cause a given molecule (such as CD40) to be increased in activity by at least 5%, 10%, 20%, 30%, 40% or more. Therefore, the agonistic effect is not necessarily 100%.

The “functional Fc region” has the “effector function” of a natural sequence Fc region. Exemplary “effector functions” include binding to C1q; CDC; binding to an Fc receptor; ADCC; phagocytosis; down-regulation of cell surface receptors (such as B cell receptor; BCR), etc. Such effector functions generally require an Fc region to be associated with a binding domain (such as an antibody variable domain) and can be evaluated using a variety of assays such as those disclosed herein.

The “effector functions” refer to those biological activities attributable to the Fc region of an antibody, which vary with the antibody isotype. Examples of antibody effector functions include: binding to C1q and complement dependent cytotoxicity (CDC); binding to an Fc receptor; antibody-dependent cell-mediated cytotoxicity (ADCC); phagocytosis; down-regulation of cell surface receptors (such as B cell receptor); and B cell activation.

The term “Fc receptor” refers to an Fc receptor that, upon binding to an immunoglobulin Fc region, triggers a signaling event that stimulates a cell carrying the receptor to perform effector functions. Fc receptors include FcγRIIIa (CD16a), FcγRI (CD64), FcγRIIa (CD32) and FcαRI (CD89).

The term “effective amount” refers to an amount or dose of the anti-CD40 antibody, conjugate or composition of the present invention which, upon administering to a patient in a single or multiple doses, produces the desired effect in the patient in need of treatment or prevention. The effective amount can be readily determined by the attending physician as a person skilled in the art with consideration of a variety of factors, such as the species of the mammal; body weight, age and general health condition; the specific disease involved; the extent or severity of the disease; the response of an individual patient; the specific antibody administered; the mode of administration; the bioavailability characteristics of the preparation administered; the selected dosing regimen; and use of any concomitant therapy.

The “therapeutically effective amount” refers to an amount effective to achieve the desired therapeutic result at a dose and for periods of time desired. The therapeutically effective amount of an antibody or an antibody fragment or a conjugate or composition thereof can vary depending on a variety of factors such as disease state, age, sex and weight of an individual, and the ability of the antibody or antibody moiety to activate a desired response in an individual. A therapeutically effective amount is also an amount in which any toxic or harmful effect of an antibody or an antibody fragment or a conjugate or composition thereof is less than a therapeutically beneficial effect. The “therapeutically effective amount” preferably inhibits measurable parameters (such as tumor growth rate and tumor volume) by at least about 20%, more preferably at least about 40%, even more preferably at least about 50%, 60% or 70% and still more preferably at least about 80% or 90% relative to untreated subjects. The ability of a compound to inhibit a measurable parameter (such as cancer) can be evaluated in an animal model system for predicting the efficacy in a human tumor.

The “prophylactically effective amount” refers to an amount effective to achieve the desired prophylactic result at a dose and for periods of time desired. Generally, since a prophylactic dose is used in a subject before or during the earlier stage of a disease, the prophylactically effective amount will be less than the therapeutically effective amount.

Generally, the “therapeutic index (TI)” refers to the ratio of median lethal dose (LD50) to median effective dose (ED50), which is an indicator of the safety of a drug.

The terms “individual” and “subject” can be used interchangeably, including mammals. Mammals include, but are not limited to, domesticated animals (e.g., cows, sheep, cats, dogs and horses), primates (e.g., humans and non-human primates such as monkeys), rabbits and rodents (e.g., mice and rats). Particularly, the individual or subject is a human.

The terms “tumor” and “cancer” can be used interchangeably herein, and encompass solid tumors and liquid tumors.

The terms “cancer” and “cancerous” refer to the physiological illness in mammals in which cell growth is unregulated.

The term “tumor” refers to all neoplastic cell growth and proliferation, whether malignant or benign, and all pre-cancerous and cancerous cells and tissues. The terms “cancer”, “cancerous” and “tumor” are not mutually exclusive when referred to herein.

The “tumor immune escape” refers to a process by which a tumor evades immune recognition and clearance. Thus, as a therapeutic concept, tumor immunity is “treated” when such evasion is attenuated, and the tumor is recognized and attacked by the immune system. Examples of tumor recognition include tumor binding, tumor shrinkage and tumor clearance.

The term “infectious disease” refers to a disease caused by a pathogen, including, for example, a viral infection, a bacterial infection, a fungal infection or a protozoal infection such as a parasitic infection.

The term “chronic infection” refers to an infection in which an infectious agent (e.g., a pathogen such as a virus, a bacterium, a protozoan such as a parasite, a fungus, or the like) has induced an immune response in an infected host but has not yet been cleared or eliminated from the host as in the course of an acute infection. Chronic infection may be persistent, latent or slow.

As used herein, the term “label” refers to a compound or a composition that is directly or indirectly conjugated or fused to a reagent, such as a polynucleotide probe or an antibody, and facilitates the detection of the reagent to which it is conjugated or fused. The label itself may be detectable (e.g., a radioisotope label or a fluorescent label), or in the case of an enzymatic label, may catalyze a detectable chemical change of a substrate compound or a composition. The term is intended to encompass direct labeling of a probe or an antibody by coupling (i.e., physically linking) a detectable substance to the probe or the antibody, and indirect labeling of a probe or an antibody by reacting with another reagent that is directly labeled. Examples of indirect labeling include detection of a primary antibody using a fluorescently-labeled secondary antibody and end labeling of a DNA probe having biotin, so that it can be detected using a fluorescently-labeled streptavidin.

The “isolated” anti-CD40 antibody refers to having been separated from components of the natural environment thereof. In some embodiments, the anti-CD40 antibody is purified to more than 95% or 99% purity, as determined by, for example, electrophoresis (e.g., SDS-PAGE, isoelectric focusing (IEF) and capillary electrophoresis) or chromatography (e.g., ion exchange or reversed-phase HPLC). For a review of methods for evaluating the purity of an antibody, see, for example, Flatman et al., J. Chromatogr. B848: 79-87 (2007).

The “isolated” nucleic acid refers to a nucleic acid molecule that has been separated from components of the natural environment thereof. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. The “isolated nucleic acid encoding anti-CD40 antibody” refers to one or more nucleic acid molecules encoding the chain or fragment of an anti-CD40 antibody, including such nucleic acid molecules in a single vector or separate vectors, and such nucleic acid molecules present at one or more locations in a host cell.

The calculation of sequence identity between sequences is performed as follows.

To determine the percent identity of two amino acid sequences or two nucleic acid sequences, the sequences are aligned for optimal comparison purposes (for example, gaps can be introduced in either or both of the first and second amino acid sequences or nucleic acid sequences for optimal alignment or non-homologous sequences can be discarded for comparison purposes). In a preferred embodiment, the length of a reference sequence aligned for comparison purposes is at least 30%, preferably at least 40%, more preferably at least 50%, 60% and even more preferably at least 70%, 80%, 90% and 100% of the length of the reference sequence. The amino acid residues or nucleotides at corresponding amino acid positions or nucleotide positions are then compared. When the position in the first sequence is occupied by the same amino acid residue or nucleotide as that at the corresponding position in the second sequence, the molecules are identical at that position.

The sequence comparison and the calculation of percent identity between two sequences can be achieved using a mathematical algorithm. In a preferred embodiment, the percent identity between two amino acid sequences is determined using the Needlema-Wunsch ((1970) J. Mol. Biol. 48:444-453) algorithm which has been incorporated into a GAP program in a GCG software package (available at http://www.gcg.com), and using a Blossum 62 matrix or a PAM250 matrix, a gap weight of 16, 14, 12, 10, 8, 6 or 4 and a length weight of 1, 2, 3, 4, 5 or 6. In another preferred embodiment, the percent identity between two nucleotide sequences is determined using the GAP program in the GCG software package (available at http://www.gcg.com) and using a NWSgapdna.CMP matrix, a gap weight of 40, 50, 60, 70 or 80 and a length weight of 1, 2, 3, 4, 5 or 6. A particularly preferred parameter set (and one parameter set that should be used unless otherwise stated) is a Blossum 62 scoring matrix with a gap penalty of 12, a gap extension penalty of 4 and a gap frameshift penalty of 5.

The percent identity between two amino acid sequences or nucleotide sequences can also be determined using a PAM120 weighted remainder table, with a gap length penalty of 12 and a gap penalty of 4, and using the E. Meyers and W. Miller algorithm ((1989) CABIOS, 4:11-17) that has been incorporated into the ALIGN program (version 2.0).

Additionally or alternatively, the nucleic acid sequences and protein sequences described herein can further be used as “query sequences” to perform searches against public databases, e.g., to identify other family member sequences or related sequences.

The term “pharmaceutical composition” refers to a composition that is present in a form which allows the active ingredients contained therein to be biologically effective and does not contain additional ingredients that would be unacceptably toxic to a subject to which the pharmaceutical composition is administered.

The term “pharmaceutical adjuvant material” refers to a diluent, an adjuvant, a carrier, an excipient, a stabilizer, etc. administered with an active substance.

As used herein, the “treatment” refers to slowing, interrupting, blocking, relieving, stopping, reducing or reversing the progression or severity of an existing symptom, disorder, condition or disease. Desirable effects of treatment include, but are not limited to, preventing occurrence or recurrence of a disease, alleviating symptoms, diminishing any direct or indirect pathological consequences of a disease, preventing metastasis, decreasing the rate of disease progression, ameliorating or palliating disease state, and relieving or improving prognosis. In some embodiments, the antibody molecule of the present invention is used to delay development of a disease or to slow the progression of a disease.

As used herein, the “prevention” includes the inhibition of the onset or development of a disease or disorder or symptoms of a particular disease or disorder. In some embodiments, a subject with a family history of cancer is a candidate for a prophylactic regimen. Generally, in the context of cancers, the term “prevention” refers to the administration of a drug prior to the onset of signs or symptoms of cancer, particularly in a subject at risk of cancer.

The term “therapeutic agent” described herein encompasses any substance effective in the prevention or treatment of tumors (e.g., cancers) and infections (e.g., chronic infections), including chemotherapeutic agents, cytotoxic agents, other antibodies, anti-infective active agents, small molecule drugs or immunomodulators.

The “chemotherapeutic agent” includes chemical compounds useful in the treatment of cancers, including but not limited to anti-tumor agents, including alkylating agents; antimetabolites; natural products; antibiotics; enzymes; miscellaneous reagents; hormones and antagonists; antiestrogens; antiandrogens; non-steroidal antiandrogens, etc. Examples of chemotherapeutic agents are those disclosed in WO 2015/153513, WO 2016/028672 or WO 2015/138920.

As used herein, the term “immunomodulator” refers to a natural or synthetic active agent or drug that inhibits or modulates an immune response. The immune response can be a humoral immune response or a cellular immune response. Immunomodulators include inhibitors of immune checkpoint molecules and activators of costimulatory molecules.

As used herein, the term “cytotoxic agent” refers to a substance that inhibits or prevents cell functions and/or causes cell death or destruction. Examples of cytotoxic agents are those disclosed in WO 2015/153513, WO 2016/028672 or WO 2015/138920.

The term “vector” when used herein refers to a nucleic acid molecule capable of propagating another nucleic acid to which it is linked. The term includes the vector as a self-replicating nucleic acid structure and the vector incorporated into the genome of a host cell into which it has been introduced. Some vectors are capable of directing the expression of nucleic acids to which they are effectively linked. Such vectors are referred to herein as “expression vectors”.

The term “host cell” refers to a cell into which an exogenous polynucleotide is introduced, including the progeny of such cells. Host cells include “transformant” and “transformed cell,” which include primary transformed cells and progeny derived therefrom regardless of the number of passages. The progeny may be not completely identical to the parent cell in terms of nucleic acid content, but may contain mutations. The mutant progeny that has the same function or biological activity screened or selected in the original transformed cell is included herein. Host cells are any type of cellular system that can be used to produce the antibody molecule of the present invention, including eukaryotic cells such as mammalian cells, insect cells and yeast cells; and prokaryotic cells, for example, E. coli cells. Host cells include cultured cells, and also include cells inside transgenic animals, transgenic plants or cultured plant tissues or animal tissues.

The “subject/patient sample” refers to a collection of cells, tissues or bodily fluids obtained from a patient or subject. The source of a tissue or cell sample may be a solid tissue, such as from a fresh, frozen and/or preserved organ, a tissue sample, a biopsy sample or a puncture sample; blood or any component of blood; body fluids, such as cerebrospinal fluid, amniotic fluid (liquor amnii), peritoneal fluid (ascites) or interstitial fluid; and cells from any time of pregnancy or development of a subject. Tissue samples may contain compounds that are not naturally intermixed with tissues in nature, such as preservatives, anticoagulants, buffers, fixatives, nutrients and antibiotics. Examples of tumor samples herein include, but are not limited to, tumor biopsies, fine needle aspirates, bronchial lavage fluid, pleural fluid (hydrothorax), sputum, urine, surgical specimens, circulating tumor cells, blood serum, blood plasma, circulating plasma proteins, ascites, primary cell cultures or cell lines derived from tumors or exhibiting tumor-like properties, and preserved tumor samples such as formalin-fixed, paraffin-embedded or frozen tumor samples.

### II. Anti-CD40 Antibody of the Present Invention

The present invention provides a new generation of an anti-human CD40 antibody that can reduce or avoid adverse events caused by traditional CD40 agonists.

The anti-CD40 antibody of the present invention comprises a light chain variable region and a heavy chain variable region. The light chain variable region comprising three complementarity determining regions, i.e., LCDR1, LCDR2 and LCDR3, respectively, and the heavy chain variable region comprising three complementarity determining regions, i.e., HCDR1, HCDR2 and HCDR3, respectively, wherein:
(a) LCDR1 comprises an amino acid sequence as shown in SEQ ID NO: 1 or a variant of SEQ ID NO: 1 having no more than 2 or no more than 1 amino acid change;
(b) LCDR2 comprises an amino acid sequence selected from any one of amino acid sequences of SEQ ID NOs: 2, 5, 6 and 7, or a variant of any one of amino acid sequences of SEQ ID NOs: 2, 5, 6 and 7 having no more than 2 or no more than 1 amino acid change;
(c) LCDR3 comprises an amino acid sequence selected from any one of amino acid sequences of SEQ ID NOs: 3 and 4, or a variant of any one of amino acid sequences of SEQ ID NOs: 3 and 4 having no more than 2 or no more than 1 amino acid change;
(d) HCDR1 comprises an amino acid sequence selected from any one of amino acid sequences of SEQ ID NOs: 8, 12, 14 and 20, or a variant of any one of amino acid sequences of SEQ ID NOs: 8, 12, 14 and 20 having no more than 2 or no more than 1 amino acid change;
(e) HCDR2 comprises an amino acid sequence selected from any one of amino acid sequences of SEQ ID NOs: 9 and 21, or a variant of any one of amino acid sequences of SEQ ID NOs: 9 and 21 having no more than 2 or no more than 1 amino acid change;
and (f) HCDR3 comprises an amino acid sequence selected from any one of amino acid sequences of SEQ ID NOs: 10, 11, 13, 15, 16, 17, 18 and 19, or a variant of any one of amino acid sequences of SEQ ID NOs: 10, 11, 13, 15, 16, 17, 18 and 19 having no more than 2 or no more than 1 amino acid change, wherein the amino acid change is an amino acid addition, deletion or substitution, for example, the amino acid change is a conservative amino acid substitution.

In some embodiments, the anti-CD40 antibody of the present invention comprises:
LCDR1 comprising an amino acid sequence RSSQGIRSSLA (SEQ ID NO: 1), LCDR2 comprising an amino acid sequence GX₁SSLX₂X₃ (SEQ ID NO: 56), and LCDR3 comprising an amino acid sequence QQLX₄SFPST (SEQ ID NO: 57), wherein X₁ is A or G, X₂ is E or L, X₃ is G or V, and X₄ is N or A; and
HCDR1 comprising an amino acid sequence GFTX₅GSYEMX₆ (SEQ ID NO: 58), HCDR2 comprising an amino acid sequence YISSX₇GETTD (SEQ ID NO: 59), and HCDR3 comprising an amino acid sequence DVFFFX₈X₉SX₁₀X₁₁X₁₂X₁₃ AYGMDV (SEQ ID NO: 60), wherein X₅ is F, A or P, X₆ is N or D, X₇ is S or A, X₈ is D or S, X₉ is S or P, X₁₀ is G or R, X₁₁ is D, P, S or F, X₁₂ is P, Nor R, and X₁₃ is G or H.

In some embodiments, the anti-CD40 antibody of the present invention comprises a light chain variable region and a heavy chain variable region, wherein:
(i) the light chain variable region comprises any one of amino acid sequences of SEQ ID NOs: 23, 29, 33, 39 and 53, and the heavy chain variable region comprises any one of amino acid sequences of SEQ ID NOs: 25, 31, 35, 37, 41, 43, 45, 47, 49, 51 and 55; or
(ii) the light chain variable region comprises an amino acid sequence having at least 85%, 90%, 91%, 92%, 93%, 94%, 95%, 96%, 97%, 98% or 99% identity to any one of amino acid sequences of SEQ ID NOs: 23, 29, 33, 39 and 53, and the heavy chain variable region comprises an amino acid sequence having at least 85%, 90%, 91%, 92%, 93%, 94%, 95%, 96%, 97%, 98% or 99% identity to any one of amino acid sequences of SEQ ID NOs: 25, 31, 35, 37, 41, 43, 45, 47, 49, 51 and 55.

In some embodiments, the anti-CD40 antibody of the present invention comprises an immunoglobulin Fc region. In some embodiments, the anti-CD40 antibody of the present invention is linked to an immunoglobulin Fc region at the C-terminus of its Fab fragment, optionally via an amino acid linker, for example, via an amino acid linker between 1 and 20 amino acids in length. In some embodiments, at least 90% of the amino acid linkers are glycine and/or serine. In some embodiments, the Fc region is from IgG, such as IgG1, IgG2, IgG3 or IgG4. In some embodiments, the Fc region is from IgG1. In some embodiments, the Fc region is from human IgG1.

In some embodiments of the present invention, the amino acid change described herein includes an amino acid substitution, addition or deletion. Preferably, the amino acid change described herein is a substitution of an amino acid, more preferably a conservative substitution.

In some embodiments, the amino acid change of the present invention occurs in a region outside a CDR (such as in an FR). In some embodiments, the substitution is a conservative substitution. The term “conservative substitution” refers to the substitution of an amino acid by another amino acid within the same class, such as the substitution of an acidic amino acid by another acidic amino acid, the substitution of a basic amino acid by another basic amino acid, or the substitution of a neutral amino acid by another neutral amino acid.

In some embodiments, the anti-CD40 antibody provided herein is altered to increase or decrease the extent to which it is glycosylated. The addition or deletion of glycosylation sites to an anti-CD40 antibody can be conveniently achieved by altering the amino acid sequence to create or remove one or more glycosylation sites. When an anti-CD40 antibody comprises an Fc region, the glycosylation of the Fc region can also be altered. In some applications, it may be useful to remove unwanted glycosylation site modifications, for example to remove fucose modifications to increase antibody-dependent cell-mediated cytotoxicity (ADCC) function (see Shield et al., (2002) JBC 277: 26733). In other applications, galactosylation modifications may be performed to modulate complement dependent cytotoxicity (CDC).

In some embodiments, a glycosylation site in a CH2 domain of an immunoglobulin Fc region of the anti-CD40 antibody of the present invention is removed, for example, the glycosylation site is removed by mutating N297 residue in a CH2 domain of a human IgG Fc region, e.g., mutating N297 residue to Gly, Ala, Gln, Asp or Glu, preferably mutating N297 residue to Gln.

The anti-CD40 antibody of the present invention is capable of binding to CD40, such as human CD40, cynomolgus monkey CD40 and mouse CD40, with a high affinity, for example, binding to CD40 with an affinity of about 10⁻⁷ M to about 10⁻¹⁰ M, as measured by ForteBio kinetic binding assay. In some embodiments, the anti-CD40 antibody of the present invention binds to a human CD40 antigen with an affinity of about 0.5×10⁻⁸ to 2×10⁻⁸ M. The anti-CD40 antibody of the present invention has a cross-reactivity to cynomolgus monkey CD40 and mouse CD40.

The anti-CD40 antibody of the present invention enhances an immune response to an antigen (such as a tumor-associated antigen (TAA)), for example, by enhancing B cell-mediated immune responses, B cell activation and/or cytokine production.

In one embodiment, the anti-CD40 antibody of the present invention is capable of enhancing an immune response to an antigen and inducing antibody-dependent cell-mediated cytotoxicity (ADCC) to a CD40-expressing cell (such as a CD40-expressing tumor cell). In addition, the anti-CD40 antibody of the present invention induces apoptosis as measured by increased expression of CD95.

In another embodiment, the anti-CD40 antibody of the present invention is capable of enhancing an immune response to an antigen without inducing antibody-dependent cell-mediated cytotoxicity (ADCC) to a CD40-expressing cell and/or complement dependent cytotoxicity (CDC) to a CD40-expressing cell. Therefore, the anti-CD40 antibody of the present invention can also exert effects on tumor cells that do not express CD40 or infectious diseases by enhancing an immune response to an antigen, for example, by enhancing a B cell-mediated immune response.

In one embodiment, the anti-CD40 antibody of the present invention is modified to comprise a constant region without effector function. For example, when the anti-CD40 antibody of the present invention comprises an Fc region, the effector function of the anti-CD40 antibody of the present invention can be reduced or eliminated by mutating the glycosylation site N297 residue in the Fc region into Gly, Ala, Gln, Asp, Glu, etc. In addition, when the anti-CD40 antibody of the present invention comprises an Fc region, modifications that reduce the binding affinity of the anti-CD40 antibody of the present invention for FcγRIIIA (CD16A) can also be included in the Fc region for reducing or eliminating effector function caused by the Fc region. In one embodiment, the modification is in the CH2 domain of the Fc region, for example, at position 329 (EU numbering) of the heavy chain (such as P329G). In one embodiment, the anti-CD40 antibody of the present invention comprises amino acid replacements at positions 234 and 235 (EU numbering) of the heavy chain. In a particular embodiment, the amino replacements are L234A and L235A (also referred to as “LALA mutation”).

In one embodiment, the anti-CD40 antibody of the present invention is capable of enhancing an immune response independent of the binding of the antibody to an Fc receptor. For example, the anti-CD40 antibody of the present invention can exhibit effective CD40 agonistic characteristics without crosslinking to an Fc receptor such as FcγR. Such agonistic characteristics include, for example, those determined by measuring an increase in B cell activity and/or an increase in B cell activation.

In another embodiment, the anti-CD40 antibody of the present invention enhances the binding of CD40 to CD40L (CD154) on a CD40-expressing cell. In particular embodiments, the anti-CD40 antibody of the present invention enhances the binding of soluble CD40L to a CD40-expressing cell by at least about 10%, 15%, 20%, 25%, 30%, 35%, 40%, 45% and 50%. In particular embodiments, the anti-CD40 antibody enhances the binding of soluble CD40L to a CD40-expressing cell by at least about 50% as measured, for example, by FACS, biolayer interferometry (BLI) or Biacore.

In some embodiments, when the anti-CD40 antibody of the present invention is incubated with Ramos cells, the expression of CD95 is induced. In some embodiments, when the anti-CD40 antibody of the present invention is incubated with human B cells, the proliferation of B cells is increased. In some embodiments, when the anti-CD40 antibody of the present invention is incubated with dendritic cells, the secretion of IL-12 and the expression of CD83 are increased.

It is expected that upon administration to a subject, the anti-CD40 antibody of the present invention will not cause adverse reactions such as dose-limiting toxicity, cytokine release syndrome (CRS) and hepatotoxicity associated with elevated concentrations of circulating liver enzyme alanine aminotransferase (ALT), aspartate aminotransferase (AST) and glutamate dehydrogenase (GLDH), or cause poor adverse reactions.

### III. Immunoconjugates

The present invention also relates to the anti-CD40 antibody (“immunoconjugate”) of the present invention conjugated to other substances. In some embodiments, the other substance is, for example, a therapeutic agent (such as a cytotoxic agent). Cytotoxic agents include any agents that are harmful to cells. Examples of cytotoxic agents (such as chemotherapeutic agents) suitable for forming immunoconjugates are known in the art. For example, the cytotoxic agents include, but are not limited to: radioisotopes; growth inhibitors; toxins such as small molecule toxins, or enzymatically active toxins of bacterial, fungal, plant or animal origin, including fragments and/or variants thereof; and various known anti-tumor or anti-cancer agents.

With regard to examples of cytotoxic agents (such as chemotherapeutic agents) suitable for forming immunoconjugates, see also, e.g., WO 2015/153513 or WO 2015/138920.

The anti-CD40 antibody of the present invention can also be linked to a solid-phase support which is particularly useful for immunoassay or for the purification of a target antigen. Such solid-phase supports include, but are not limited to, glass, cellulose, polyacrylamide, nylon, polystyrene, polyvinyl chloride or polypropylene.

In some embodiments, the immunoconjugate is used for preventing or treating tumors. In some embodiments, the tumors are cancers. In some embodiments, the immunoconjugate is used for preventing or treating infections, for example, chronic infections, such as bacterial infections, viral infections, fungal infections and protozoal infections.

### IV. Nucleic Acid of the Present Invention and Host Cell Comprising Same

In one aspect, the present invention provides a nucleic acid encoding any of the above anti-CD40 antibody or the fragment thereof or any one of the chain thereof. In one embodiment, a vector containing the nucleic acid is provided. In one embodiment, the vector is an expression vector. In one embodiment, a host cell containing the nucleic acid or the vector is provided. In one embodiment, the host cell is a eukaryotic cell. In another embodiment, the host cell is selected from a yeast cell, a mammalian cell (such as CHO cell or 293 cell), or other cells suitable for the preparation of antibodies or antigen-binding fragments thereof. In another embodiment, the host cell is a prokaryotic cell.

For example, the nucleic acid of the present invention comprises a nucleic acid encoding any one of amino acid sequences as shown in SEQ ID NOs: 23, 25, 29, 31, 33, 35, 37, 39, 41, 43, 45, 47, 49, 51, 53 and 55, or a nucleic acid encoding an amino acid sequence having at least 85%, 90%, 91%, 92%, 93%, 94%, 95%, 96%, 97%, 98% or 99% identity to any one of amino acid sequences as shown in SEQ ID NOs: 23, 25, 29, 31, 33, 35, 37, 39, 41, 43, 45, 47, 49, 51, 53 and 55.

The present invention also encompasses nucleic acids that hybridize under stringent conditions with the following nucleic acids, or nucleic acids that encode a polypeptide sequence having one or more amino acid substitutions (such as conservative substitutions), deletions or insertions as compared to the following nucleic acids: a nucleic acid comprising a nucleic acid sequence encoding any one of amino acid sequences as shown in SEQ ID NOs: 23, 25, 29, 31, 33, 35, 37, 39, 41, 43, 45, 47, 49, 51, 53 and 55; or a nucleic acid comprising a nucleic acid sequence encoding an amino acid sequence having at least 85%, 90%, 91%, 92%, 93%, 94%, 95%, 96%, 97%, 98% or 99% identity to any one of amino acid sequences as shown in SEQ ID NOs: 23, 25, 29, 31, 33, 35, 37, 39, 41, 43, 45, 47, 49, 51, 53 and 55.

In one embodiment, one or more vectors comprising the nucleic acid is provided. In one embodiment, the vector is an expression vector, for example, a eukaryotic expression vector. Vectors include, but are not limited to, viruses, plasmids, cosmids, λ phages or yeast artificial chromosomes (YAC). In one embodiment, the vector is a pcDNA3.3 vector.

Once an expression vector or a DNA sequence has been prepared for expression, the expression vector can be transfected or introduced into a suitable host cell. Various techniques, such as protoplast fusion, calcium phosphate precipitation, electroporation, retroviral transduction, viral transfection, gene gun, lipid-based transfection or other conventional techniques, can be used to achieve this purpose. Methods and conditions for culturing the resulting transfected cells and for recovering the resulting antibody molecules are known to a person skilled in the art and can be varied or optimized on the basis of the present description and methods known in the prior art, depending on the particular expression vector and mammalian host cell used.

Additionally, cells that have stably incorporated DNA into their chromosomes can be selected by introducing one or more markers that allow selection of transfected host cells. Markers may, for example, provide prototrophy, biocidal resistance (such as antibiotics) or resistance to heavy metals (such as copper) to auxotrophic hosts. A selectable marker gene can be directly linked to a DNA sequence to be expressed or introduced into the same cell by co-transformation. Additional elements may also be required for optimally synthesis of mRNA. These elements may include splicing signals, as well as transcription promoters, enhancers and termination signals.

In one embodiment, a host cell containing the polynucleotide of the present invention is provided. In some embodiments, a host cell containing the expression vector of the present invention is provided. In some embodiments, the host cell is selected from a yeast cell, a mammalian cell or other cells suitable for the preparation of antibodies. Suitable host cells include prokaryotic microorganisms, such as E. coli. Host cells can also be eukaryotic microorganisms such as filamentous fungi or yeasts, or various eukaryotic cells such as insect cells. Vertebrate cells can also be used as hosts. For example, mammalian cell lines engineered to be adapted to grow in suspension can be used. Examples of useful mammalian host cell lines include monkey kidney CV1 line transformed by SV40 (COS-7); and human embryonic kidney lines (HEK 293 or 293F cells), 293 cells, baby hamster kidney cells (BHK), monkey kidney cells (CV1), African green monkey kidney cells (VERO-76), human cervical cancer cells (HELA), canine kidney cells (MDCK), Buffalo rat liver cells (BRL 3A), human lung cells (W138), human liver cells (Hep G2), Chinese hamster ovary cells (CHO cells), CHOS cells, NSO cells, and myeloma cell lines such as Y0, NS0, P3X63 and Sp2/0. In a preferred embodiment, the host cell is a CHO cell or a 293 cell.

### V. Production and Purification of Anti-CD40 Antibody of the Present Invention

In one embodiment, the present invention provides a method for preparing an anti-CD40 antibody, wherein the method comprises culturing a host cell containing a nucleic acid encoding the anti-CD40 antibody or an expression vector containing the nucleic acid under conditions suitable for the expression of the nucleic acid encoding the anti-CD40 antibody, and optionally isolating the anti-CD40 antibody. In a certain embodiment, the method further comprises recovering the anti-CD40 antibody from the host cell (or a host cell culture medium).

For recombinant production of the anti-CD40 antibody of the present invention, a nucleic acid encoding the anti-CD40 antibody of the present invention is first isolated and inserted into a vector for further cloning and/or expression in a host cell. Such nucleic acids are readily isolated and sequenced using a conventional procedure, e.g., using an oligonucleotide probe that is capable of specifically binding to a nucleic acid encoding the anti-CD40 antibody of the present invention.

The anti-CD40 antibody of the present invention prepared as described herein can be purified by techniques known in the prior art, such as high performance liquid chromatography, ion exchange chromatography, gel electrophoresis, affinity chromatography and steric exclusion chromatography. The actual conditions used to purify a particular protein also depend on factors such as net charge, hydrophobicity and hydrophilicity, which will be apparent to a person skilled in the art. The purity of the anti-CD40 antibody of the present invention can be determined by any of a variety of well-known analytical methods, including steric exclusion chromatography, gel electrophoresis, high performance liquid chromatography, etc.

### VI. Activity Assay for Anti-CD40 Antibody of the Present Invention

The anti-CD40 antibody provided herein can be identified, screened or characterized for its physical/chemical properties and/or biological activities by a variety of assays known in the art. In one aspect the anti-CD40 antibody of the present invention is tested for its binding activity to an antigen, for example, by known methods such as ELISA and Western blotting. The binding to CD40 can be determined using methods known in the art, and exemplary methods are disclosed herein. In some embodiments, the binding of the anti-CD40 antibody of the present invention to CD40 is determined using SPR or biolayer interferometry.

The present invention also provides an assay for identifying an anti-CD40 antibody having biological activity. Biological activities can include, for example, binding to CD40 (e.g., human CD40, monkey CD40, mouse CD40) on the surface of cells, enhancing the CD40/CD40L binding, activating antigen presenting cells, inducing CD40-expressing tumor cells to express CD95 and enhancing B cell-mediated immune responses.

Cells for use in any of the above in vitro assays include cell lines that naturally expressing CD40 or are engineered to express CD40. The cell line engineered to express CD40 is a cell line that normally does not express CD40, but expresses CD40 upon transfection of DNA encoding CD40 into the cell.

It can be understood that any of the above assays can be performed by replacing an anti-CD40 antibody with the immunoconjugate of the present invention.

### VII. Pharmaceutical Composition and Pharmaceutical Preparation

In some embodiments, the present invention provides a composition comprising any of the anti-CD40 antibodies or immunoconjugates thereof described herein, preferably the composition is a pharmaceutical composition. In one embodiment, the composition further comprises a pharmaceutical adjuvant material. In one embodiment, the composition (such as a pharmaceutical composition) comprises a combination of the anti-CD40 antibody or the immunoconjugate thereof of the present invention, and one or more other therapeutic agents, such as chemotherapeutic agents, cytotoxic agents, other antibodies, anti-infective active agents, small molecule drugs or immunomodulators, preferably anti-PD-1 antibodies or anti-PD-L1 antibodies.

In some embodiments, the composition is used for preventing or treating tumors. In some embodiments, the tumors are cancers. In some embodiments, the composition is used for preventing or treating infections, for example, chronic infections, such as bacterial infections, viral infections, fungal infections and protozoal infections.

The present invention also includes a composition (including a pharmaceutical composition or a pharmaceutical preparation) comprising an anti-CD40 antibody or an immunoconjugate thereof and/or a composition (including a pharmaceutical composition or a pharmaceutical preparation) comprising a polynucleotide encoding an anti-CD40 antibody. These compositions may also comprise suitable pharmaceutical adjuvant materials, such as pharmaceutical carriers and pharmaceutical excipients known in the art, including buffers.

As used herein, the “pharmaceutical carrier” includes any and all solvents, dispersion media, isotonic agents, absorption delaying agents, etc. that are physiologically compatible. Pharmaceutical carriers suitable for use in the present invention can be sterile liquids, such as water and oils, including those of petroleum, animal, vegetable or synthetic origin, such as peanut oil, soybean oil, mineral oil and sesame oil. Water is a preferred carrier when the pharmaceutical composition is administered intravenously. Saline solutions, aqueous dextrose and glycerol solutions can also be used as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, etc. For using excipients and the use of excipients, reference can also be made to “Handbook of Pharmaceutical Excipients”, fifth edition, R. C. Rowe, P. J. Seskey and S. C. Owen, Pharmaceutical Press, London, Chicago. The composition, if desired, can also contain small amounts of wetting agents or emulsifying agents, or pH buffers. These compositions can be in the form of solutions, suspensions, emulsions, tablets, pills, capsules, powders, sustained-release preparations, etc. Oral preparation can contain a standard pharmaceutical carrier and/or excipient such as pharmaceutical grade mannitol, lactose, starch, magnesium stearate and saccharin.

The pharmaceutical preparation comprising the anti-CD40 antibody described herein can be prepared by mixing the anti-CD40 antibody of the present invention having the desired purity with one or more optional pharmaceutical adjuvant materials (Remington's Pharmaceutical Sciences, 16th edition, Osol, A. ed. (1980)), preferably in the form of a lyophilized preparation or an aqueous solution.

The pharmaceutical compositions or preparation of the present invention may also contain more than one active ingredient, which is required for the particular indication being treated, preferably those having complementary activities that do not adversely affect each other. For example, it is desirable to also provide other anti-cancer active ingredients or anti-infective active ingredients, such as chemotherapeutic agents, cytotoxic agents, other antibodies, anti-infective active agents, small molecule drugs or immunomodulators, such as anti-PD-1 antibodies and anti-PD-L1 antibodies. Such active ingredients are suitably present in combination in an amount effective for the intended use.

Sustained-release preparations can be prepared. Suitable examples of sustained-release preparations include semipermeable matrices of solid hydrophobic polymers containing the anti-CD40 antibody of the present invention, which matrices are in the form of shaped articles such as films or microcapsules.

### VIII. Combined Products or Kits

In some embodiments, the present invention also provides a combined product, comprising the anti-CD40 antibody or antigen-binding fragment or immunoconjugate thereof of the present invention, and one or more other therapeutic agents (such as chemotherapeutic agents, other antibodies, cytotoxic agents, anti-infective active agents, small molecule drugs or immunomodulators). In some embodiments, other antibodies are, for example, anti-PD-1 antibodies and anti-PD-L1 antibodies.

In some embodiments, the combined product is used for preventing or treating tumors. In some embodiments, the tumors are cancers, etc.

In some regiments, two or more ingredients of the combined product may be sequentially, separately or simultaneously administered in combination to a subject.

In some embodiments, the present invention also provides a kit comprising the anti-CD40 antibody, pharmaceutical composition, immunoconjugate or combined product of the present invention, and optionally a package insert directing administration.

In some embodiments, the present invention also provides a pharmaceutical product comprising the anti-CD40 antibody, pharmaceutical composition, immunoconjugate or combined product of the present invention, optionally further comprising a package insert directing administration.

### IX. Use of Anti-CD40 Antibody of the Present Invention

In one aspect, the present invention relates to a method for modulating an immune response in an individual. The method comprises administering to a subject an effective amount of the anti-CD40 antibody, or the pharmaceutical composition, immunoconjugate or combined product comprising the anti-CD40 antibody disclosed herein, thereby modulating an immune response in the subject. In one embodiment, a therapeutically effective amount of the anti-CD40 antibody, pharmaceutical composition, immunoconjugate or combined product disclosed herein restores, enhances, stimulates or increases an immune response in a subject.

In some embodiments, the present invention relates to a method for increasing the activity of CD40, enhancing the binding of CD40 to CD40L and inducing the secretion of cytokines such as IL-12 in an individual, wherein the method comprises administering to a subject an effective amount of the anti-CD40 antibody or the pharmaceutical composition, immunoconjugate or combined product comprising same disclosed herein.

In another aspect, the present invention relates to a method for preventing or treating tumors (such as cancers) in a subject, wherein the method comprises administering to the subject an effective amount of the anti-CD40 antibody, or the pharmaceutical composition, immunoconjugate or combined product comprising same disclosed herein. In some embodiments, the tumor is a tumor immune escape. In some embodiments, the tumor is a cancer.

In another aspect, the present invention relates to a method for causing antibody-dependent cell-mediated cytotoxicity in a subject, wherein the method comprises administering to the subject an effective amount of the anti-CD40 antibody, or the pharmaceutical composition, immunoconjugate or combined product comprising same disclosed herein.

The subject may be a mammal, for example, a primate, preferably a higher primate, for example, human (such as a patient suffering from or at risk of suffering from the disease described herein). In one embodiment, the subject suffers from or is at risk of suffering from the disease described herein (such as the tumor or infectious disease described herein). In certain embodiments, the subject receives or has received other treatments such as chemotherapy treatment and/or radiation therapy. Alternatively or in combination, the subject is immunocompromised by an infection or is at risk of being immunocompromised by an infection.

In some embodiments, the tumors, such as cancers, as described herein include but are not limited to solid tumors, hematological cancers, soft tissue tumors and metastatic lesions.

Examples of solid tumors include malignant tumors, such as sarcomas and cancers (including adenocarcinomas and squamous cell carcinomas) of the various organ systems, such as those that invade liver, lung, breast, lymph, gastrointestinal tract (such as colon), pancreas, genitourinary tract (such as kidney and bladder epithelial cells), prostate and pharynx. Adenocarcinomas include malignant tumors such as most colon cancers, rectal cancers, renal cell carcinomas, liver cancer, non-small cell lung cancer, small intestine cancer and esophageal cancer. Squamous cell carcinomas include malignant tumors, such as those in the lung, esophagus, skin, head and neck regions, oral cavity, anus and cervix. In one embodiment, the cancer is melanoma, for example, advanced melanoma. In one embodiment, the cancer is a lymphoma, a renal cell carcinoma, a non-small cell lung cancer, a liver cancer, a pancreatic cancer, a colon adenocarcinoma or a breast cancer. Metastatic lesions of the aforementioned cancers can also be treated or prevented using the method and composition of the present invention.

Non-limiting examples of preferred cancers to be treated include lymphoma (e.g., diffuse large B-cell lymphoma, Hodgkin lymphoma and non-Hodgkin lymphoma), breast cancer (e.g., metastatic breast cancer), liver cancer (e.g., hepatocellular carcinoma (HCC)), lung cancer (e.g., non-small cell lung cancer (NSCLC), e.g., stage IV or recurrent non-small cell lung cancer, NSCLC adenocarcinoma, or NSCLC squamous cell carcinoma), myeloma (e.g., multiple myeloma), leukemia (e.g., chronic myelogenous leukemia), skin cancer (e.g., melanoma (e.g., stage III or IV melanoma), or Merkel cell carcinoma), head and neck cancer (e.g., head and neck squamous cell carcinoma (HNSCC)), myelodysplastic syndrome, bladder cancer (e.g., transitional cell carcinoma), renal carcinoma (e.g., renal cell carcinoma, e.g., clear cell renal cell carcinoma, e.g., advanced or metastatic clear cell renal cell carcinoma), and colon cancer. Additionally, refractory or recurrent malignant tumors (e.g., pancreatic cancer) can be treated using the anti-CD40 antibody, or the pharmaceutical composition, immunoconjugate or combined product comprising same described herein.

In some embodiments, the anti-CD40 antibody of the present invention binds to CD40 on the surface of B cells or antigen presenting cells to create a cascade effect, which activates T cells, thereby treating tumors.

In some embodiments, the anti-CD40 antibody of the present invention treat tumors with the overexpression of CD40 on the surface by ADCC, CTL and/or induction of tumor cell apoptosis.

In some embodiments, the anti-CD40 antibody, or the immunoconjugate, composition or combined product comprising same of the present invention may delay the onset of a disorder and/or symptoms associated with a disorder.

In some embodiments, the prevention or treatment method described herein further comprises administering to a subject or an individual the anti-CD40 antibody, pharmaceutical composition, immunoconjugate or combined product disclosed herein in combination with one or more other therapies, e.g., therapy methods and/or other therapeutic agents.

In some embodiments, the therapy methods include surgery (such as tumor resection), radiation therapy (e.g., an external beam therapy, which involves three-dimensional conformal radiation therapy in which an area of irradiation is designed), localized irradiation (e.g., irradiation directed at a preselected target or organ) or focused irradiation), etc. The focused irradiation may be selected from stereotactic radiosurgery, fractionated stereotactic radiosurgery and intensity modulated radiotherapy. The focused irradiation may have a radiation source selected from a particle beam (proton), cobalt-60 (photon) and a linear accelerator (X-ray), for example, as described in WO 2012/177624.

Radiation therapy can be administered by one or a combination of several methods including, but not limited to, external beam therapy, internal radiation therapy, implant irradiation, stereotactic radiosurgery, systemic radiation therapy, radiotherapy and permanent or transient interstitial brachytherapy.

In some embodiments, the therapeutic agent is selected from a chemotherapeutic agent, a cytotoxic agent, other antibodies, an anti-infective active agent, a small molecule drug or an immunomodulator (e.g., an activator of a costimulatory molecule or an inhibitor of an immune checkpoint molecule).

Exemplary other antibodies include, but are not limited to, inhibitors of immune checkpoint molecules (e.g., anti-PD-1, anti-PD-L1, anti-TIM-3, anti-CEACAM or anti-LAG-3 antibodies), and antibodies that stimulate immune cells (e.g., agonistic anti-GITR antibodies or anti-CD137 antibodies). Preferably, the other antibodies are selected from anti-PD-1 antibodies and/or anti-PD-L1 antibodies. More preferably, the anti-PD-1 antibody is Nivolumab from Bristol-Myers Squibb Company (BMS) and Pembrolizumab from Merck; and the anti-PD-L1 antibody is Atezolizumab developed by Roche, Avelumab developed cooperatively by Merck KGaA and Pfizer, and Durvalumab developed by AstraZeneca.

In some embodiments, the immunomodulator is an activator or agonist of a costimulatory molecule. In one embodiment, the agonist of the costimulatory molecule is selected from an agonist (e.g., an agonistic antibody or an antigen-binding fragment thereof, or a soluble fusion) of the following molecules: OX40, CD2, CD27, CD28, CDS, ICAM-1, LFA-1 (CD11a/CD18), ICOS (CD278), 4-1BB (CD137), GITR, CD30, BAFFR, HVEM, CD7, LIGHT, NKG2C, SLAMF7, NKp80, CD160, B7-H3 or CD83 ligand.

Combination therapies of the present invention encompasses combined administration (in which two or more therapeutic agents are contained in the same preparation or separate preparations) and separate administration. In the case of separate administration, the administration of the anti-CD40 antibody or immunoconjugate of the present invention, etc. may be performed prior to, simultaneously with and/or after administration of other therapies.

In one embodiment, the administration of anti-CD40 antibodies and the administration of other therapies (e.g., therapy methods or therapeutic agents) occur within about one month, within about one, two or three weeks, or within about 1, 2, 3, 4, 5, or 6 days of each other.

The anti-CD40 antibody (and pharmaceutical composition or immunoconjugate comprising same) of the present invention can be administered by any suitable method, including parenteral administration, intrapulmonary administration and intranasal administration, and, if topical treatment is desired, intralesional administration. Parenteral infusion includes intramuscular, intravenous, intraarterial, intraperitoneal or subcutaneous administration. Administration can be performed by any suitable route, for example, by injection, such as intravenous or subcutaneous injection, depending to a certain extent on whether dosing is short-term or long-term. A variety of dosing schedules are encompassed herein, including but not limited to single administration, multiple administrations at multiple time points, bolus administration and pulse infusion.

To prevent or treat diseases, suitable doses of the anti-CD40 antibody of the present invention (when used alone or in combination with one or more other therapeutic agents) will depend on the type of a disease to be treated, the type of the anti-CD40 antibody, the severity and progression of the disease, whether the anti-CD40 antibody is administered for prophylactic or therapeutic purposes, previous treatment, the clinical history and response to the anti-CD40 antibody of a patient, and the judgment of the attending physician. The anti-CD40 antibody is suitably administered to a patient in one treatment or a series of treatments. The dose and treatment regimen of the anti-CD40 antibody can be determined by the skilled person.

It can be understood that any of the above preventions or treatments can be performed by replacing the anti-CD40 antibody with the immunoconjugate, composition or combined product of the present invention.

### X. Methods and Compositions for Diagnosis and Detection

In certain embodiments, any of the anti-CD40 antibodies provided herein can be used to detect the presence of CD40 in a biological sample. As used herein, the term “detect” includes quantitative or qualitative detection. Exemplary detection methods may involve immunohistochemistry, immunocytochemistry, flow cytometry (e.g., FACS), magnetic beads complexed with antibody molecules and ELISA. In certain embodiments, the biological sample is blood, blood serum, or other body fluid samples of biological origin. In certain embodiments, the biological sample comprises a cell or a tissue. In some embodiments, the biological sample is from a hyperproliferative or cancerous lesion.

In one embodiment, an anti-CD40 antibody for use in a diagnosis or detection method is provided. In another aspect, a method for detecting the presence of CD40 in a biological sample is provided. In certain embodiments, the method comprises detecting the presence of CD40 protein in a biological sample. In certain embodiments, the CD40 is human CD40. In certain embodiments, the method comprises contacting the biological sample with the anti-CD40 antibody as described herein under conditions that allow the anti-CD40 antibody to bind to CD40, and detecting whether a complex is formed between the anti-CD40 antibody and CD40. The formation of the complex indicates the presence of CD40. The method may be an in vitro or in vivo method. In one embodiment, the anti-CD40 antibody is used to select a subject suitable for being treated with the anti-CD40 antibody, e.g., wherein the CD40 is a biomarker for selecting the subject.

In one embodiment, the anti-CD40 antibody of the present invention can be used to diagnose cancers or tumors, for example, to evaluate (e.g., monitor) the treatment or progression, diagnosis and/or staging of the disease described herein (e.g., a hyperproliferative or cancerous disease) in a subject. In certain embodiments, the labeled anti-CD40 antibody is provided. Labels include, but are not limited to, labels or moieties that are detected directly (such as fluorescent labels, chromophore labels, electron-dense labels, chemiluminescent labels and radioactive labels), and moieties that are detected indirectly (such as enzymes or ligands), for example, by an enzymatic reaction or a molecular interaction. Exemplary labels include, but are not limited to, radioisotopes ³²P, ¹⁴C, ¹²⁵I, ³H and ¹³¹I, fluorophores such as rare earth chelates or luciferin and derivatives thereof, rhodamine and derivatives thereof, dansyl, umbelliferone, luciferase, such as firefly luciferase and bacterial luciferase (U.S. Pat. No. 4,737,456), fluorescein, 2,3-dihydrophthalazinedione, horseradish peroxidase (HRP), alkaline phosphatase, β-galactosidase, glucoamylase, lysozyme, carbohydrate oxidase (such as glucose oxidase, galactose oxidase and glucose-6-phosphate dehydrogenase), heterocyclic oxidase (such as uricase and xanthine oxidase), enzymes that oxidize dye precursors with hydrogen peroxide (such as HR, lactoperoxidase or microperoxidase), biotin/avidin, spin labels, phage labels, stable free radicals, etc.

In some embodiments of any of the inventions provided herein, the samples are obtained prior to treatment with the anti-CD40 antibody. In some embodiments, the sample is obtained after the cancer has metastasized. In some embodiments, the sample is formalin fixed paraffin embedded (FFPE). In some embodiments, the sample is a biopsy (e.g., a core biopsy), a surgical specimen (e.g., a specimen from a surgical resection) or a fine needle aspirate.

In some embodiments, the CD40 is detected prior to treatment, e.g., prior to initiation of treatment or prior to certain treatment after a treatment interval.

In some embodiments, a method for treating tumors or infections is provided, comprising: detecting the presence of CD40 in a subject (e.g., a sample) (e.g., a subject sample containing cancer cells), thereby determining a value of CD40; comparing the value of CD40 with a control value (e.g., a value of CD40 in a sample of a healthy individual); and if the value of CD40 is less than the control value, administering to the subject a therapeutically effective amount of an anti-CD40 antibody (e.g., the anti-CD40 antibody described herein), optionally in combination with one or more other therapies, thereby treating tumors or infections.

It can be understood that various embodiments described in various sections of the present invention, such as diseases, therapeutic agents, therapy methods and administration, are equally applicable to, or may be combined with, embodiments of other sections of the present invention. Embodiments described in various sections of the present invention, such as properties, uses and methods, applicable to anti-CD40 antibodies are equally applicable to compositions, conjugates, combined products, kits, etc. comprising the anti-CD40 antibodies.

### XI. Sequences of Exemplary Anti-CD40 Antibody of the Present Invention

The sequences of the exemplary anti-CD40 antibodies of the present invention are as shown in the tables below.

TABLE 1
CDR sequences in light chain variable domains (VL) of exemplary
antibodies of the present invention (defined by AbM numbering)

| Antibody | | | | | | |
|---|---|---|---|---|---|---|
| name | SEQ ID NO: | LCDR1 | SEQ ID NO: | LCDR2 | SEQ ID NO: | LCDR3 |
| C8-WT | 1 | RSSQGIRSSLA | 2 | GASSLEG | 3 | QQLNSFPST |
| C8-1 | 1 | RSSQGIRSSLA | 2 | GASSLEG | 4 | QQLASFPST |
| C8-2 | 1 | RSSQGIRSSLA | 2 | GASSLEG | 3 | QQLNSFPST |
| C8-3 | 1 | RSSQGIRSSLA | 5 | GASSLEV | 3 | QQLNSFPST |
| C8-4 | 1 | RSSQGIRSSLA | 2 | GASSLEG | 3 | QQLNSFPST |
| C8-5 | 1 | RSSQGIRSSLA | 6 | GGSSLEG | 3 | QQLNSFPST |
| C8-6 | 1 | RSSQGIRSSLA | 2 | GASSLEG | 3 | QQLNSFPST |
| C8-7 | 1 | RSSQGIRSSLA | 2 | GASSLEG | 3 | QQLNSFPST |
| C8-8 | 1 | RSSQGIRSSLA | 2 | GASSLEG | 3 | QQLNSFPST |
| C8-9 | 1 | RSSQGIRSSLA | 2 | GASSLEG | 3 | QQLNSFPST |
| C8-10 | 1 | RSSQGIRSSLA | 2 | GASSLEG | 3 | QQLNSFPST |
| C8-11 | 1 | RSSQGIRSSLA | 7 | GASSLLG | 3 | QQLNSFPST |
| Consensus | 1 | RSSQGIRSSLA | 56 | GX₁SSLX₂X₃ | 57 | QQLX₄SFPST |
| sequence | | | | | | |

TABLE 2
CDR sequences in heavy chain variable domains (VH) of exemplary
antibodies of the present invention (defined by AbM numbering)

| Antibody | SEQ | | SEQ | | SEQ | |
|---|---|---|---|---|---|---|
| name | ID NO: | CDR1 | ID NO: | CDR2 | ID NO: | CDR3 |
| C8-WT | 8 | GFTFGSYEMN | 9 | YISSSGETTD | 10 | DVFFFDSSGDNGAYGMDV |
| C8-1 | 8 | GFTFGSYEMN | 9 | YISSSGETTD | 10 | DVFFFDSSGDNGAYGMDV |
| C8-2 | 8 | GFTFGSYEMN | 9 | YISSSGETTD | 11 | DVFFFSPSGDNGAYGMDV |
| C8-3 | 12 | GFTAGSYEMN | 9 | YISSSGETTD | 10 | DVFFFDSSGDNGAYGMDV |
| C8-4 | 8 | GFTFGSYEMN | 9 | YISSSGETTD | 13 | DVFFFDSSGPRGAYGMDV |
| C8-5 | 14 | GFTFGSYEMD | 9 | YISSSGETTD | 10 | DVFFFDSSGDNGAYGMDV |
| C8-6 | 8 | GFTFGSYEMN | 9 | YISSSGETTD | 15 | DVFFFDSSGSPGAYGMDV |
| C8-7 | 8 | GFTFGSYEMN | 9 | YISSSGETTD | 16 | DVFFFDSSGDNHAYGMDV |
| C8-8 | 8 | GFTFGSYEMN | 9 | YISSSGETTD | 17 | DVFFFDSSGPPGAYGMDV |
| C8-9 | 8 | GFTFGSYEMN | 9 | YISSSGETTD | 18 | DVFFFDSSRPNGAYGMDV |
| C8-10 | 8 | GFTFGSYEMN | 9 | YISSSGETTD | 19 | DVFFFDSSGFPGAYGMDV |
| C8-11 | 20 | GFTPGSYEMN | 21 | YISSAGETTD | 10 | DVFFFDSSGDNGAYGMDV |
| Consensus | 58 | GFTX₅GSYEMX₆ | 59 | YISSX₇GETTD | 60 | DVFFFX₈X₉SX₁₀X₁₁X₁₂X₁₃AYGMDV |
| sequence | | | | | | |

TABLE 3
Nucleotide sequences and amino acid sequences of variable
regions of exemplary antibodies of the present invention

| Antibody | | |
|---|---|---|
| variable | | |
| region name | Amino acid sequence | Nucleotide sequence |
| C8-WT-VL | DIQLTQSPSSLTASV | GACATCCAGTTGACCCAGTCTCCATCCTCCCTGA |
| | GDRVTITCRSSQGIR | CTGCATCTGTCGGAGACAGAGTCACCATTACTTG |
| | SSLAWYQQKPGKPP | CCGGTCAAGTCAGGGCATTCGCAGTTCTTTAGCCT |
| | KLLIYGASSLEGGVP | GGTATCAACAGAAACCAGGGAAACCTCCTAAGCT |
| | SRFSGSGSGTDFTLTI | CCTGATCTATGGTGCCTCCAGTTTGGAAGGTGGGG |
| | SSLQPEDFATYYCQ | TCCCATCAAGGTTCAGCGGCAGTGGATCTGGGAC |
| | QLNSFPSTFGQGTKL | AGATTTCACTCTCACCATCAGTAGTCTGCAGCCTG |
| | EIK (SEQ ID NO: 23) | AAGATTTTGCAACTTATTACTGTCAGCAGCTCAAT |
| | | AGTTTCCCGTCCACTTTTGGCCAGGGGACCAAGCT |
| | | GGAAATCAAA (SEQ ID NO: 22) |
| C8-WT-VH | EVQLLESGGGLVRP | GAGGTGCAGCTGTTGGAGTCTGGGGGAGGCTTGG |
| | GGSLRLSCAASGFTF | TACGGCCTGGAGGGTCCCTGAGACTCTCCTGTGCA |
| | GSYEMNWVRQAPG | GCCTCTGGATTCACCTTCGGTAGTTATGAAATGAA |
| | KGLEWVSYISSSGET | TTGGGTCCGCCAGGCTCCCGGGAAGGGGCTGGAG |
| | TDYADSVKGRFTVS | TGGGTCTCATATATAAGTAGCAGTGGTGAAACGA |
| | RDNSKNLLYLQMNS | CCGACTACGCAGACTCTGTGAAGGGCCGCTTCAC |
| | LRAEDTAVYYCARD | CGTCTCCAGAGACAACAGTAAGAATTTACTGTAT |
| | VFFFDSSGDNGAYG | CTGCAAATGAACAGCCTGAGAGCCGAGGACACGG |
| | MDVWGQGTMVTVS | CTGTATATTACTGTGCGAGAGACGTGTTTTTTTTT |
| | S (SEQ ID NO: 25) | GATAGTAGTGGTGACAATGGGGCCTACGGTATGG |
| | | ACGTCTGGGGCCAAGGGACAATGGTCACCGTCTC |
| | | ATCA (SEQ ID NO: 24) |
| C8-1-VL | DIQLTQSPSSLTASV | GACATCCAGTTGACCCAGTCTCCATCCTCCCTGA |
| | GDRVTITCRSSQGIR | CTGCATCTGTCGGAGACAGAGTCACCATTACTTG |
| | SSLAWYQQKPGKPP | CCGGTCAAGTCAGGGCATTCGCAGTTCTTTAGCCT |
| | KLLIYGASSLEGGVP | GGTATCAACAGAAACCAGGGAAACCTCCTAAGCT |
| | SRFSGSGSGTDFTLTI | CCTGATCTATGGTGCCTCCAGTTTGGAAGGTGGGG |
| | SSLQPEDFATYYCQ | TCCCATCAAGGTTCAGCGGCAGTGGATCTGGGAC |
| | QLASFPSTFGQGTKL | AGATTTCACTCTCACCATCAGTAGTCTGCAGCCTG |
| | EIK (SEQ ID NO: 29) | AAGATTTTGCAACTTATTACTGTCAGCAGCTTGCT |
| | | AGTTTCCCGTCCACTTTTGGCCAGGGGACCAAGCT |
| | | GGAAATCAAA (SEQ ID NO: 28) |
| C8-1-VH | Identical to C8-WT-VH | |
| C8-2-VL | Identical to C8-WT-VL | |
| C8-2-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMNWVRQAPGKGL | GCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFSPSG | CGCTTCACCGTCTCCAGAGACAACAGTAAGAAT |
| | DNGAYGMDVWGQGT | TTACTGTATCTGCAAATGAACAGCCTGAGAGCC |
| | MVTVSS (SEQ ID NO: | GAGGACACGGCTGTATATTACTGTGCGAGAGAC |
| | 31) | GTGTTTTTTTTTTCGCCGAGTGGTGACAATGGGG |
| | | CCTACGGTATGGACGTCTGGGGCCAAGGGACAA |
| | | TGGTCACCGTCTCATCA (SEQ ID NO: 30) |
| C8-3-VL | DIQLTQSPSSLTASVG | GACATCCAGTTGACCCAGTCTCCATCCTCCCTGA |
| | DRVTITCRSSQGIRSSL | CTGCATCTGTCGGAGACAGAGTCACCATTACTT |
| | AWYQQKPGKPPKLLI | GCCGGTCAAGTCAGGGCATTCGCAGTTCTTTAG |
| | YGASSLEVGVPSRFSG | CCTGGTATCAACAGAAACCAGGGAAACCTCCTA |
| | SGSGTDFTLTISSLQPE | AGCTCCTGATCTATGGTGCCTCCAGTTTGGAAGT |
| | DFATYYCQQLNSFPST | GGGGGTCCCATCAAGGTTCAGCGGCAGTGGATC |
| | FGQGTKLEIK (SEQ ID | TGGGACAGATTTCACTCTCACCATCAGTAGTCTG |
| | NO: 33) | CAGCCTGAAGATTTTGCAACTTATTACTGTCAGC |
| | | AGCTCAATAGTTTCCCGTCCACTTTTGGCCAGGG |
| | | GACCAAGCTGGAAATCAAA (SEQ ID NO: 32) |
| C8-3-VH | EVQLLESGGGLVRPG | GAGGTGCAGCTGTTGGAGTCTGGGGGAGGCTTG |
| | GSLRLSCAASGFTAGS | GTACGGCCTGGAGGGTCCCTGAGACTCTCCTGT |
| | YEMNWVRQAPGKGL | GCAGCCTCTGGATTCACCGCGGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCTCTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GDNGAYGMDVWGQ | TTACTGTATCTGCAAATGAACAGCCTGAGAGCC |
| | GTMVTVSS (SEQ ID | GAGGACACGGCTGTATATTACTGTGCGAGAGAC |
| | NO: 35) | GTGTTTTTTTTTGATAGTAGTGGTGACAATGGGG |
| | | CCTACGGTATGGACGTCTGGGGCCAAGGGACAA |
| | | TGGTCACCGTCTCATCA (SEQ ID NO: 34) |
| C8-4-VL | Identical to C8-WT-VL | |
| C8-4-VH | EVQLLESGGGLVRPG | GAGGTGCAGCTGTTGGAGTCTGGGGGAGGCTT |
| | GSLRLSCAASGFTFGS | GGTACGGCCTGGAGGGTCCCTGAGACTCTCCTG |
| | YEMNWVRQAPGKGL | TGCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GPRGAYGMDVWGQG | TTACTGTATCTGCAAATGAACAGCCTGAGAGCC |
| | TMVTVSS (SEQ ID NO: | GAGGACACGGCTGTATATTACTGTGCGAGAGAC |
| | 37) | GTGTTTTTTTTTGATAGTAGTGGTCCTCGGGGGG |
| | | CCTACGGTATGGACGTCTGGGGCCAAGGGACAA |
| | | TGGTCACCGTCTCATCA (SEQ ID NO: 36) |
| C8-5-VL | DIQLTQSPSSLTASVG | GACATCCAGTTGACCCAGTCTCCATCCTCCCTGA |
| | DRVTITCRSSQGIRSS | CTGCATCTGTCGGAGACAGAGTCACCATTACTT |
| | LAWYQQKPGKPPKL | GCCGGTCAAGTCAGGGCATTCGCAGTTCTTTAG |
| | LIYGGSSLEGGVPSRF | CCTGGTATCAACAGAAACCAGGGAAACCTCCTA |
| | SGSGSGTDFTLTISSLQ | AGCTCCTGATCTATGGTGGGTCCAGTTTGGAAG |
| | PEDFATYYCQQLNSFP | GTGGGGTCCCATCAAGGTTCAGCGGCAGTGGAT |
| | STFGQGTKLEIK (SEQ | CTGGGACAGATTTCACTCTCACCATCAGTAGTCT |
| | ID NO: 39) | GCAGCCTGAAGATTTTGCAACTTATTACTGTCAG |
| | | CAGCTCAATAGTTTCCCGTCCACTTTTGGCCAGG |
| | | GGACCAAGCTGGAAATCAAA (SEQ ID NO: 38) |
| C8-5-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMDWVRQAPGKGL | GCAGCCTCTGGATTCACCTTCGGTAGTTATGAA |
| | EWVSYISSSGETTDYA | ATGGATTGGGTCCGCCAGGCTCCCGGGAAGGGG |
| | DSVKPRFTVSRDNSK | CTGGAGTGGGTCTCATATATAAGTAGCAGTGGT |
| | NLLYLQMNSLRAEDT | GAAACGACCGACTACGCAGACTCTGTGAAGCCG |
| | AVYYCARDVFFFDSS | CGCTTCACCGTCTCCAGAGACAACAGTAAGAAT |
| | GDNGAYGMDVWGQ | TTACTGTATCTGCAAATGAACAGCCTGAGAGCC |
| | GTMVTVSS (SEQ ID | GAGGACACGGCTGTATATTACTGTGCGAGAGAC |
| | NO: 41) | GTGTTTTTTTTTGATAGTAGTGGTGACAATGGGG |
| | | CCTACGGTATGGACGTCTGGGGCCAAGGGACAA |
| | | TGGTCACCGTCTCATCA (SEQ ID NO: 40) |
| C8-6-VL | Identical to C8-WT-VL | |
| C8-6-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMNWVRQAPGKGL | GCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GSPGAYGMDVWGQG | TTACTGTATCTGCAAATGAACAGCCTGAGAGCC |
| | TMVTVSS (SEQ ID NO: | GAGGACACGGCTGTATATTACTGTGCGAGAGAC |
| | 43) | GTGTTTTTTTTTGATAGTAGTGGTTCTCCGGGGG |
| | | CCTACGGTATGGACGTCTGGGGCCAAGGGACA |
| | | ATGGTCACCGTCTCATCA (SEQ ID NO: 42) |
| C8-7-VL | Identical to C8-WT-VL | |
| C8-7-VH | EVQLLESGGGLVRPG | GAGGTGCAGCTGTTGGAGTCTGGGGGAGGCTT |
| | GSLRLSCAASGFTFGS | GGTACGGCCTGGAGGGTCCCTGAGACTCTCCTG |
| | YEMNWVRQAPGKGL | TGCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GDNHAYGMDVWGQ | TTACTGTATCTGCAAATGAACAGCCTGAGAGCC |
| | GTMVTVSS (SEQ ID | GAGGACACGGCTGTATATTACTGTGCGAGAGAC |
| | NO: 45) | GTGTTTTTTTTTGATAGTAGTGGTGACAATCATG |
| | | CGTACGGTATGGACGTCTGGGGCCAAGGGACAA |
| | | TGGTCACCGTCTCATCA (SEQ ID NO: 44) |
| C8-8-VL | Identical to C8-WT-VL | |
| C8-8-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMNWVRQAPGKGL | GCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GPPGAYGMDVWGQG | TTACTGTATCTGCAAATGAACAGCCTGAGAGCC |
| | TMVTVSS (SEQ ID NO: | GAGGACACGGCTGTATATTACTGTGCGAGAGAC |
| | 47) | GTGTTTTTTTTTGATAGTAGTGGTCCTCCTGGGG |
| | | CCTACGGTATGGACGTCTGGGGCCAAGGGACA |
| | | ATGGTCACCGTCTCATCA (SEQ ID NO: 46) |
| C8-9-VL | Identical to C8-WT-VL | |
| C8-9-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMNWVRQAPGKGL | GCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | RPNGAYGMDVWGQG | TTACTGTATCTGCAAATGAACAGCCTGAGAGCC |
| | TMVTVSS (SEQ ID NO: | GAGGACACGGCTGTATATTACTGTGCGAGAGAC |
| | 49) | GTGTTTTTTTTTGATAGTAGTCGTCCGAATGGGG |
| | | CCTACGGTATGGACGTCTGGGGCCAAGGGACAA |
| | | TGGTCACCGTCTCATCA (SEQ ID NO: 48) |
| C8-10-VL | Identical to C8-WT-VL | |
| C8-10-VH | EVQLLESGGGLVRPG | GAGGTGCAGCTGTTGGAGTCTGGGGGAGGCTTG |
| | GSLRLSCAASGFTFGS | GTACGGCCTGGAGGGTCCCTGAGACTCTCCTGT |
| | YEMNWVRQAPGKGL | GCAGCCTCTGGATTCACCTTCGGTAGTTATGAA |
| | EWVSYISSSGETTDYA | ATGAATTGGGTCCGCCAGGCTCCCGGGAAGGGG |
| | DSVKGRFTVSRDNSK | CTGGAGTGGGTCTCATATATAAGTAGCAGTGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GFPGAYGMDVWGQG | TTACTGTATCTGCAAATGAACAGCCTGAGAGCC |
| | TMVTVSS (SEQ ID NO: | GAGGACACGGCTGTATATTACTGTGCGAGAGAC |
| | 51) | GTGTTTTTTTTTGATAGTAGTGGTTTTCCTGGGG |
| | | CCTACGGTATGGACGTCTGGGGCCAAGGGACA |
| | | ATGGTCACCGTCTCATCA (SEQ ID NO: 50) |
| C8-11-VL | DIQLTQSPSSLTASVG | GACATCCAGTTGACCCAGTCTCCATCCTCCCTGA |
| | DRVTITCRSSQGIRSS | CTGCATCTGTCGGAGACAGAGTCACCATTACTT |
| | LAWYQQKPGKPPKL | GCCGGTCAAGTCAGGGCATTCGCAGTTCTTTAG |
| | LIYGASSLLGGVPSRF | CCTGGTATCAACAGAAACCAGGGAAACCTCCTA |
| | SGSGSGTDFTLTISSLQ | AGCTCCTGATCTATGGTGCCTCCAGTTTGTTGGG |
| | PEDFATYYCQQLNSFP | TGGGGTCCCATCAAGGTTCAGCGGCAGTGGATC |
| | STFGQGTKLEIK (SEQ | TGGGACAGATTTCACTCTCACCATCAGTAGTCTG |
| | ID NO: 53) | CAGCCTGAAGATTTTGCAACTTATTACTGTCAGC |
| | | AGCTCAATAGTTTCCCGTCCACTTTTGGCCAGGG |
| | | GACCAAGCTGGAAATCAAA (SEQ ID NO: 52) |
| C8-11- | EVQLLESGGGLVRPG | GAGGTGCAGCTGTTGGAGTCTGGGGGAGGCTTG |
| VH | GSLRLSCAASGFTPGS | GTACGGCCCGGAGGGTCCCTGAGACTCTCCTGT |
| | YEMNWVRQAPGKGL | GCAGCCTCTGGATTCACCCCTGGTAGTTATGAA |
| | EWVSYISSAGETTDYA | ATGAATTGGGTCCGCCAGGCTCCCGGGAAGGGG |
| | DSVKGRFTVSRDNSK | CTGGAGTGGGTCTCATATATAAGTAGCGCGGGT |
| | NLLYLQMNSLRAEDT | GAAACGACCGACTACGCAGACTCTGTGAAGGGC |
| | AVYYCARDVFFFDSS | CGCTTCACCGTCTCCAGAGACAACAGTAAGAAT |
| | GDNGAYGMDVWGQ | TTACTGTATCTGCAAATGAACAGCCTGAGAGCC |
| | GTMVTVSS (SEQ ID | GAGGACACGGCTGTATATTACTGTGCGAGAGAC |
| | NO: 55) | GTGTTTTTTTTTGATAGTAGTGGTGACAATGGGG |
| | | CCTACGGTATGGACGTCTGGGGCCAAGGGACAA |
| | | TGGTCACCGTCTCATCA (SEQ ID NO: 54) |

The following examples are described to assist in the understanding of the present invention. The examples are not intended and should not be construed to limit the scope of protection of the present invention in any way.

### EXAMPLES

### Example 1: Preparation of Anti-CD40 Antibody

### 1.1. Preparation of P17-Fc Antigen

A human CD40 extracellular domain (CD40 ECD) (positions 21-193 of Uniprot: P25942) gene fragment was fused to the N-terminus of the DNA sequence of a human IgG1 Fc fragment, and a nucleotide sequence encoding the CD40 ECD-Fc fusion protein was synthesized and cloned into the multiple cloning site of a pcDNA3.3 (Invitrogen) vector to obtain an expression vector expressing the CD40 ECD-Fc fusion protein, also referred to herein as a P17-Fc fusion protein expression vector.

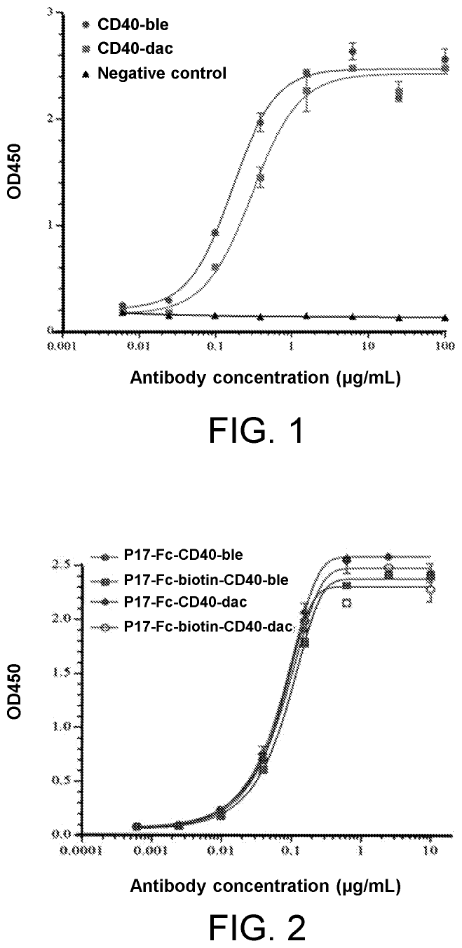

The P17-Fc fusion protein expression vector was introduced into CHO-K cells (Thermo Fisher) for eukaryotic expression (see “Therapeutic Fc-Fusion Proteins” (Steven M. Chamow et al.), pages 45-62, the content related to “Methods of Production for Fc-Fusion Proteins”, for detailed preparation steps) to obtain a P17-Fc fusion protein. The immunogenicity of the prepared P17-Fc fusion protein was verified by determining the binding of the P17-Fc fusion protein to anti-CD40 antibodies Dacetuzumab (see EP 1885399 B1, hereinafter referred to as CD40-Dac) and Bleslumab (see US 20100234578, hereinafter referred to as CD40-Ble) by standard ELISA methods. As shown in FIG. 1, the P17-Fc fusion protein exhibited an affinity for Dacetuzumab and Bleslumab.

### 1.2. Biotinylation of P17-Fc Fusion Protein

The P17-Fc fusion protein was labeled with a calculated amount of EZ-Link Sulfo-NHS-LC-biotin using an EZ-Link™ Sulfo-NHS-LC-Biotinylation Kit (Thermo Fisher, Cat. No. 21435) according to the manufacturer's instructions. The amount of the biotin-labeled P17-Fc fusion protein (also referred to herein as “P17-Fc-biotin” and “biotinylated P17-Fc fusion protein”) was measured by a biotin quantification kit (Thermo Fisher, 28005).

Further, the conformation of the biotinylated P17-Fc fusion protein was verified by ELISA for Dacetuzumab and Bleslumab using the same method as that in example 1.1.

As shown in FIG. 2, compared to the binding of a unbiotinylated P17-Fc fusion protein to Dacetuzumab and Bleslumab, the biotinylated P17-Fc fusion protein showed similar binding to these two anti-CD40 antibodies.

IL-23-Fc-biotin (IL-23 sequence was derived from Uniprot: Q9NPF7) was prepared by a similar method for removing Fc-binding phages in the subsequent screening of an antibody library.

P17-His protein (P17 sequence was identical to positions 21-193 of Uniprot: P25942) was prepared by a similar method, with a 6-histidine tag at the C-terminus.

### 1.3. Screening of Antibody Library

See patent application CN 202010236256.8 for the method for constructing and screening a fully humanized antibody library. A fully humanized antibody phage display library was screened using the biotinylated P17-Fc fusion protein of example 1.1 to obtain an anti-CD40 Fab antibody fragment displayed on phage that specifically binds to human CD40.

The specific screening method was as follows. An antibody library was screened using a magnetic bead method. Before each round of panning of the fully humanized antibody phage display library by using the biotinylated P17-Fc fusion protein of example 1.1, Fc-binding phages were removed by panning the antibody library with the IL-23-Fc-biotin of example 1.1, and then the phage single clones capable of specifically binding to human CD40 protein were screened with the biotinylated P17-Fc fusion protein. After removing non-specifically adsorbed phages by washing, the resulting phages were collected, amplified using E. coli SS320 cells, and then used for the next round of panning.

In each round of panning, the ratio of input phage to output phage was calculated, which was used as an indicator of CD40-specific phage enrichment.

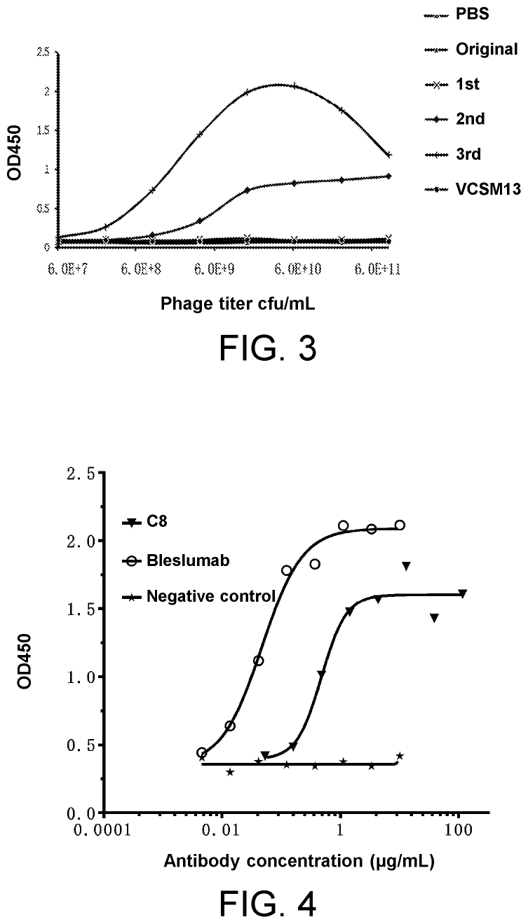

Enrichment of CD40 by each library was also confirmed by phage library ELISA, in which CD40-binding phages were highly enriched after three rounds of panning, as shown in FIG. 3.

The binding of the screened phage Fab to P17-Fc-biotin and IL23-Fc-biotin was tested. Phage clones exhibiting specific affinity only for P17-Fc-biotin were selected, DNA was extracted, and sequencing was performed.

One phage clone C8 containing a unique Fab sequence was obtained, with nucleotide and amino acid sequences encoding same as shown below.

Nucleotide sequence encoding C8 Fab light chain
variable region (C8-WT-VL)
(SEQ ID NO: 22)
GACATCCAGTTGACCCAGTCTCCATCCTCCCTGACTGCATCTGTCGGAG
ACAGAGTCACCATTACTTGCCGGTCAAGTCAGGGCATTCGCAGTTCTTT
AGCCTGGTATCAACAGAAACCAGGGAAACCTCCTAAGCTCCTGATCTAT
GGTGCCTCCAGTTTGGAAGGTGGGGTCCCATCAAGGTTCAGCGGCAGTG
GATCTGGGACAGATTTCACTCTCACCATCAGTAGTCTGCAGCCTGAAGA
TTTTGCAACTTATTACTGTCAGCAGCTCAATAGTTTCCCGTCCACTTTT
GGCCAGGGGACCAAGCTGGAAATCAAA
Amino acid sequence encoding C8 Fab light chain
variable region (C8-WT-VL)
(SEQ ID NO: 23)
DIQLTQSPSSLTASVGDRVTITCRSSQGIRSSLAWYQQKPGKPPKLLIY
GASSLEGGVPSRFSGSGSGTDFTLTISSLQPEDFATYYCQQLNSFPSTF
GQGTKLEIK
Nucleotide sequence encoding C8 Fab heavy chain
variable region (C8-WT-VH)
(SEQ ID NO: 24)
GAGGTGCAGCTGTTGGAGTCTGGGGGAGGCTTGGTACGGCCTGGAGGGT
CCCTGAGACTCTCCTGTGCAGCCTCTGGATTCACCTTCGGTAGTTATGA
AATGAATTGGGTCCGCCAGGCTCCCGGGAAGGGGCTGGAGTGGGTCTCA
TATATAAGTAGCAGTGGTGAAACGACCGACTACGCAGACTCTGTGAAGG
GCCGCTTCACCGTCTCCAGAGACAACAGTAAGAATTTACTGTATCTGCA
AATGAACAGCCTGAGAGCCGAGGACACGGCTGTATATTACTGTGCGAGA
GACGTGTTTTTTTTTGATAGTAGTGGTGACAATGGGGCCTACGGTATGG
ACGTCTGGGGCCAAGGGACAATGGTCACCGTCTCATCA
Amino acid sequence encoding C8 Fab heavy chain
variable region (C8-WT-VH)
(SEQ ID NO: 25)
EVQLLESGGGLVRPGGSLRLSCAASGFTFGSYEMNWVRQAPGKGLEWVS
YISSSGETTDYADSVKGRFTVSRDNSKNLLYLQMNSLRAEDTAVYYCAR
DVFFFDSSGDNGAYGMDVWGQGTMVTVSS

The nucleotide sequence encoding the heavy chain variable region and the nucleotide sequence encoding the light chain variable region of the phage C8 clone were respectively linked into a pcDNA3.3 vector, expressed as Fab fragments in CHO-K cells (Thermo Fisher) and purified. The affinity of the Fab candidate molecules for P17-Fc was detected by ELISA, and the results were as shown in FIG. 4.

It can be seen from FIG. 4, the C8 clone Fab binds to CD40 with an EC50 value of about 0.4764 μg/mL, and the positive control Bleslumab binds to CD40 with an EC50 value of about 0.04701 μg/mL.

Further, to test the affinity of anti-CD40 Fab for CD40 expressed on the cell surface, CHO-K cells expressing human CD40 (Uniprot: P25942) on the cell surface and CHO-K cells expressing cynomolgus monkey CD40 (Uniprot: G7PG38) on the cell surface were prepared. Specifically, a sequence encoding human CD40 (Uniprot: P25942) was cloned into the multiple cloning site of a pcDNA3.3 (Invitrogen) vector to obtain an expression vector expressing human CD40; and then, the expression vector expressing human CD40 was introduced into CHO-K cells (Thermo Fisher) for eukaryotic expression to obtain CHO-K cells expressing human CD40 on the cell surface (hereinafter also referred to as “CHO-K-huCD40 cells”). Similarly, CHO-K cells expressing cynomolgus monkey CD40 on the cell surface (hereinafter also referred to as “CHO-K-cynoCD40 cells”) were obtained.

The CHO-K-huCD40 cells or CHO-K-cynoCD40 cells were seeded into a 96-well plate at 1.0×10⁵ cells/well, and diluted C8 anti-CD40 Fab candidate molecules were added. After incubation at 4° C. for 30 minutes, the cells were washed, added with 100 μL of APC-labeled goat anti-human IgG secondary antibody (Jackson ImmunoResearch Inc, Cat. No. 109-136-097, Allophycocyanin (APC) AffiniPure F(ab′)₂ Fragment Goat Anti-Human IgG, F(ab′)₂ fragment specific), and incubated at 4° C. for 30 minutes. Then, the cells were washed, and the binding of the C8 anti-CD40 Fab candidate molecules to CD40 molecules expressed on the cell surface was detected by flow cytometry (Beckman Coulter).

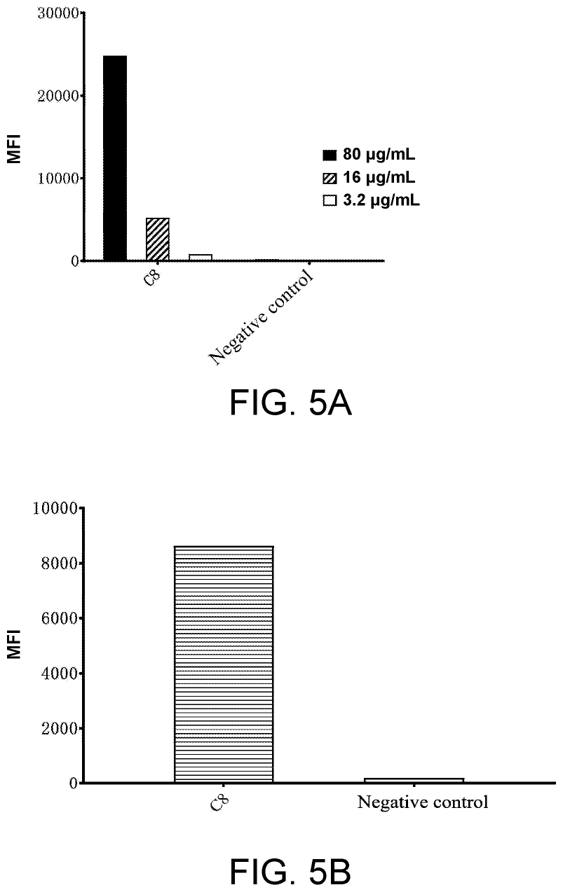

FIG. 5A shows that C8 clone anti-CD40 Fab molecules at different concentrations have the binding affinity for CHO-K-huCD40 cells, and the binding affinity is presented as C8 Fab concentration dependent in a C8 Fab concentration range of 3.2 μg/mL-80 μg/mL.

FIG. 5B shows that C8 clone anti-CD40 Fab molecules also show the binding affinity for CHO-K-cynoCD40 cells, and the C8 Fab concentration used is 80 μg/mL. It can be seen therefrom that the C8 clone Fab molecule has a cross-reactivity to cynomolgus monkey CD40.

### 1.4. Expression, Purification and Concentration Determination of Full-Length Antibody

The C8 clone Fab candidate molecule of example 1.3 was subjected to full-length antibody construction, wherein the sequence of human IgG1 Fc was linked to the C-terminus of the heavy chain sequence of the Fab molecule.

Specifically, the obtained nucleotide sequence encoding the antibody heavy chain variable region and the obtained nucleotide sequence encoding the antibody light chain variable region were constructed into an engineered eukaryotic expression vector plasmid pcDNA3.3 (Invitrogen) containing light and heavy chain constant region fragments, respectively. According to the manufacturer's instructions, the full-length heavy and light chains of the antibody were co-expressed in CHO-K cells using the ExpiCHO transient expression system (Thermo Fisher, A29133), and then purification was performed by protein A affinity chromatography to obtain a C8 clone anti-CD40 full-length antibody.

For the purified full-length antibody, the concentration was determined by absorbance A280, and the quality of the full-length antibody was verified by SDS-PAGE, differential scanning fluorimetry (DSF) and size exclusion chromatography (SEC). After passing the quality inspection, the full-length antibody was subpackaged and stored at −80° C. The specific method was described in patent application number CN 202010236256.8.

Amino acid sequence encoding C8 full-length
antibody light chain (C8-WT-LC)
(SEQ ID NO: 26)
DIQLTQSPSSLTASVGDRVTITCRSSQGIRSSLAWYQQKPGKPPKLLIY
GASSLEGGVPSRFSGSGSGTDFTLTISSLQPEDFATYYCQQLNSFPSTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Amino acid sequence encoding C8 full-length
antibody heavy chain (C8-WT-HC)
(SEQ ID NO: 27)
EVQLLESGGGLVRPGGSLRLSCAASGFTFGSYEMNWVRQAPGKGLEWVS
YISSSGETTDYADSVKGRFTVSRDNSKNLLYLQMNSLRAEDTAVYYCAR
DVFFFDSSGDNGAYGMDVWGQGTMVTVSSASTKGPSVFPLAPSSKSTSG
GTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEA
LHNHYTQKSLSLSPGK

### Example 2: Cell-Based Functional Determination of Anti-CD40 Antibody

### 2.1. CD40 Binding Properties and Species Cross-Reactivity of Candidate Anti-CD40 Full-Length Antibody

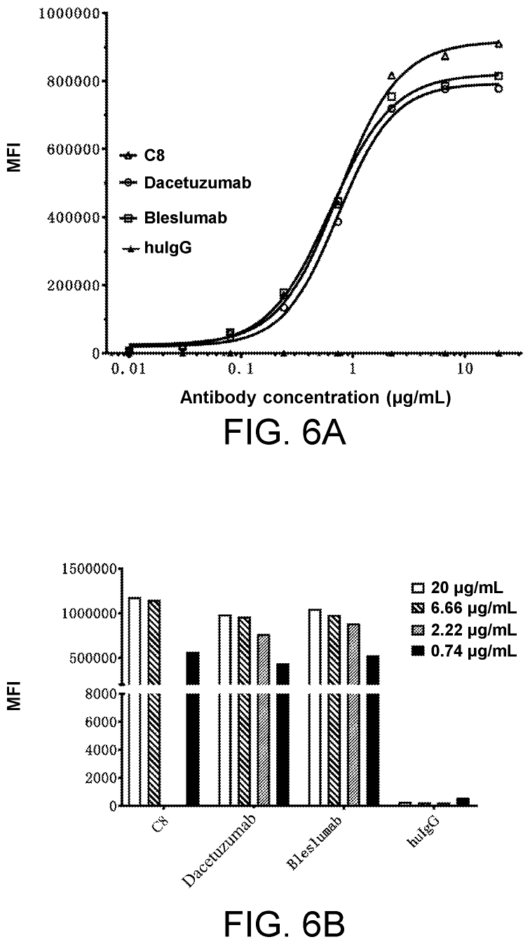

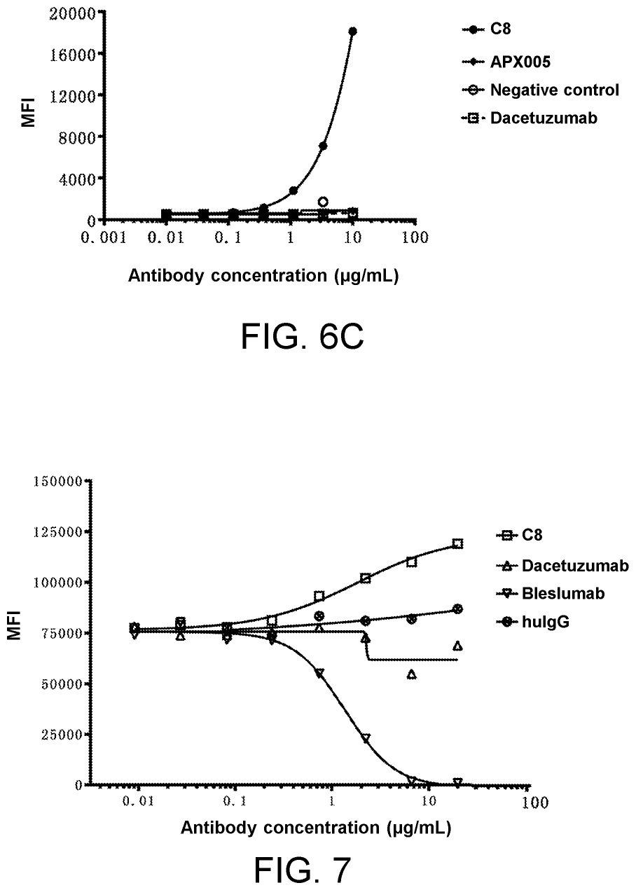

To confirm the binding activity and cross-reactivity of the full-length anti-CD40 antibody prepared in example 1.4 of the present invention to CD40, CHO-K-huCD40 cells or CHO-K-cynoCD40 cells were seeded into a 96-well plate at 1.0×10⁵ cells/well. 100 μL of diluted anti-CD40 candidate antibody prepared in example 1.4 was added to the 96-well plate. After incubation at 4° C. for 30 minutes, the cells were washed, added with 100 μL of APC-labeled anti-human IgG secondary antibody (Allophycocyanin (APC) AffiniPure Goat Anti-Human IgG, Fcγ fragment specific, Jackson ImmunoResearch Inc, Cat. No. 109-135-098), and incubated at 4° C. for 30 minutes. Then, the cells were washed, and the binding of the full-length anti-CD40 antibody to CD40 molecules expressed on the cell was detected by flow cytometry. The results were as shown in FIG. 6A and FIG. 6B.

It can be seen from FIG. 6A and FIG. 6B, after the C8 clone Fab fragment is subjected to full-length antibody construction, the C8 clone anti-CD40 full-length antibody still retains the affinity of the C8 clone Fab fragment for human CD40 and the cross-reactivity of the C8 clone Fab fragment to cynomolgus monkey CD40.

Similarly to example 1.3, CHO-K cells expressing full-length mouse CD40 (Uniprot: P27512) (hereinafter also referred to as “CHO-K-mouseCD40 cells”) were prepared. The cross-reactivity of the candidate C8 full-length anti-CD40 antibody prepared in example 1.4 to mouse CD40 was tested using the CHO-K-mouseCD40 cells. The CHO-K-mouseCD40 cells were seeded into a 96-well plate at 1.0×10⁵ cells/well. 100 μL of diluted anti-CD40 candidate antibody and two control antibodies (Dacetuzumab and APX005) were added to the 96-well plate, respectively. After incubation at 4° C. for 30 minutes, the cells were washed, added with FITC-labeled anti-human IgG Fcγ secondary antibody (AffiniPure F(ab′)₂ Fragment Goat Anti-Human IgG, Fcγ fragment specific, Jackson Immunoresearch, Cat. No. 109-006-098), and incubated for 30 minutes at 4° C. The cells were then washed and tested by flow cytometry. The results were as shown in FIG. 6C.

It can be seen from FIG. 6C that the C8 clone full-length antibody molecule exhibits the significant binding activity to mouse CD40, whereas the control antibodies Dacetuzumab and APX005 have no cross-reactivity to mouse CD40.

### 2.2. Effect of Candidate Anti-CD40 Antibody on the Binding of CD40 to CD40L

To test the effect of the anti-CD40 full-length antibody prepared in example 1.4 on the binding of CD40 to CD40L, CHO-K cells expressing human CD40 (i.e., CHO-K-huCD40 cells) were seeded into a 96-well plate at 1.0×10⁵ cells/well, and 100 μL of diluted anti-CD40 full-length antibody or isotype control (i.e., purified human IgG1 isotype control recombinant antibody (Biolegend, Cat. No. 403502)) was added to the 96-well plate. After incubation at 4° C. for 30 min, the cells were washed.

CD40L (ACRO Biosystems, Cat. No. CDL-H5248) was biotinylated according to the method as described in example 1, the biotinylated CD40L protein was diluted to 200 ng/mL, and 100 μL of the diluted biotinylated CD40L (also referred to herein as: CD40L-biotin) was added to the 96-well plate. After incubation at 4° C. for 30 min, the cells were washed, then added with 100 μL of PE-labeled streptavidin (eBioscience, Cat. No. 12-4317-87), and incubated at 4° C. for another 30 min. The cells were then washed and tested by flow cytometry.

As shown in FIG. 7, the candidate antibody C8 clone at the concentration of 0.01-20 μg/mL exhibited the enhanced CD40 binding activity to CD40L, and was an anti-CD40 antibody having potential to be CD40 agonistic. The control antibody Bleslumab significantly inhibited the binding of CD40 to CD40L, while Dacetuzumab did not affect the binding of CD40 to CD40L at a lower concentration, and inhibited the binding of CD40 to CD40L at a higher concentration.

### 2.3. Determination of Candidate Anti-CD40 Antibody Mediated Activation of Immature Dendritic Cells

Immature dendritic cells (iDCs) can be transformed into mature DC cells upon activation by anti-CD40 antibodies. In this example, the iDC activation activity mediated by the candidate antibody was evaluated by testing IL-12 cytokine secreted by mature DC cells, the expression of CD83 marker, etc.

By using a RPMI 1640 medium added with 1000 U/mL rhGM-CSF (PeproTech, Cat. No. 300-03-50) and 500 U/mL rhIL-4 (PeproTech, Cat. No. 200-04-50) and supplemented with 10% FCS, iDC cells were developed from monocytes prepared from human peripheral blood, and were then seeded into a 6-well plate at 5.0×10⁴ iDC cells/well. Diluents of the candidate anti-CD40 full-length antibody, each control antibody and CD40L (ACRO Biosystems, Cat. No. CDL-H5248) were added to the iDC cells and cultured for three days. The culture solution and cells were collected.

The IL-12 secretion in the collected culture solution and the CD83 marker expression in the collected cells were determined by ELISA or flow cytometry.

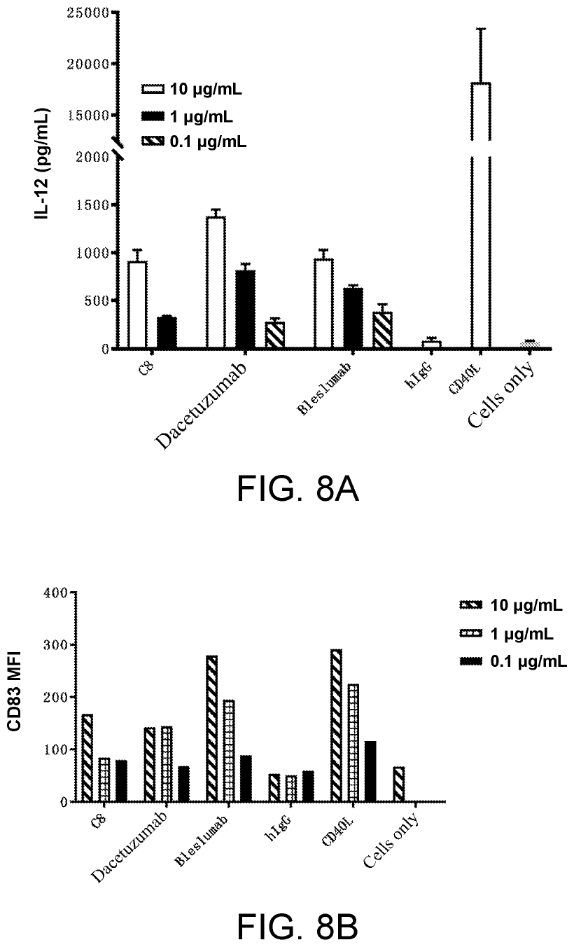

As shown in FIG. 8A and FIG. 8B, the C8 clone full-length antibody exhibited a significant and dose-dependent iDC activation activity. In this regard, similar to Dacetuzumab, the C8 clone full-length antibody was a weak CD40 agonist. CD40L, as a control, resulted in substantial IL-12 secretion.

### 2.4. Candidate Anti-CD40 Antibody-Mediated Induction of CD95 in Ramos Cells

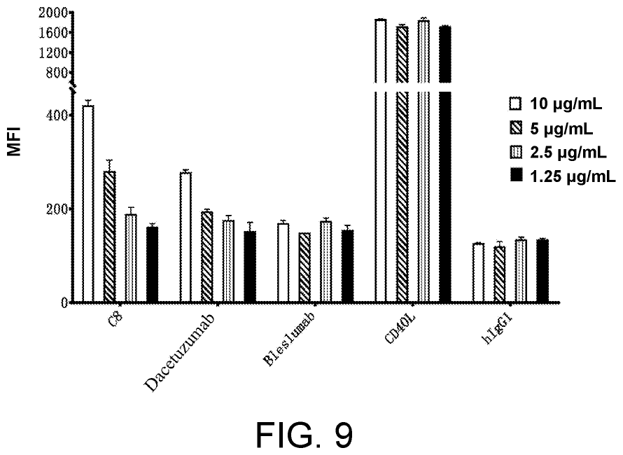

Ramos cells are human lymphoma cells that naturally express CD40 molecules on the cell surface. To test whether the anti-CD40 antibody binds to CD40 on Ramos cells and activates the downstream signaling pathway of CD40, Ramos cells (ATCC No.: CRL-1596) were plated into microtiter plate wells containing a RPMI 1640 medium supplemented with 10% fetal bovine serum (FBS) (hereinafter also referred to as “complete RPMI 1640 medium”) at 1.0×10⁵ cells/well. Diluted anti-CD40 antibodies or CD40L (10 μg/mL for the first well, 2-fold gradient dilution, 4 concentration wells, detailed concentrations as shown in FIG. 9) were added to Ramos cells and incubated overnight at 37° C. The induced CD95 expression on the Ramos cell surface was determined by flow cytometry.

As shown in FIG. 9, the C8 clone full-length antibody exhibited a dose-dependent CD95 induction activity compared to hIgG1 as a negative control, and exhibited a weak agonistic activity.

### Example 3: In Vivo Pharmacodynamic Test of Anti-CD40 Antibody

### 3.1. Inhibition of Tumor Growth in Mouse CDX Model of Ramos Cells by Anti-CD40 Agonistic Antibody

A cell derived xenograft (CDX) model is a tumor model constructed by transplanting a tumor cell line into nude mice or NSG mice. In this example, a mouse CDX model was constructed by transplanting Ramos cells subcutaneously into mice, and the anti-tumor efficacy of the candidate anti-CD40 antibody in the mouse CDX model was tested.

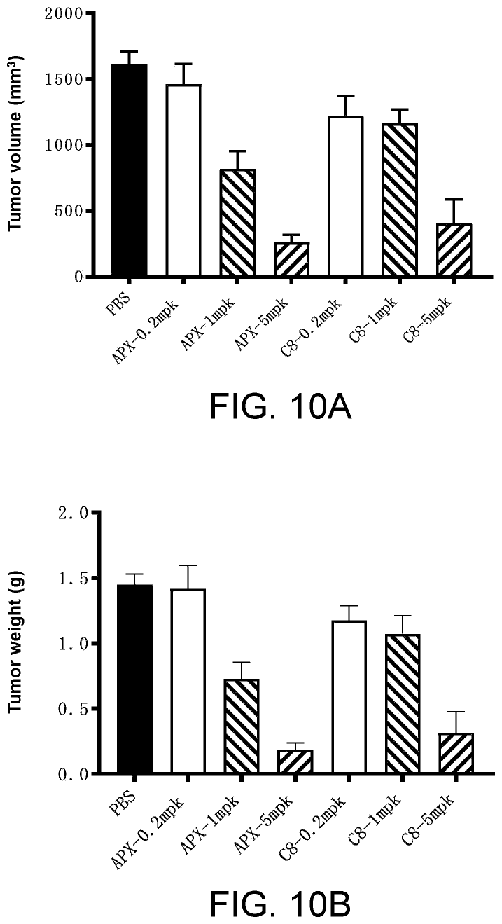

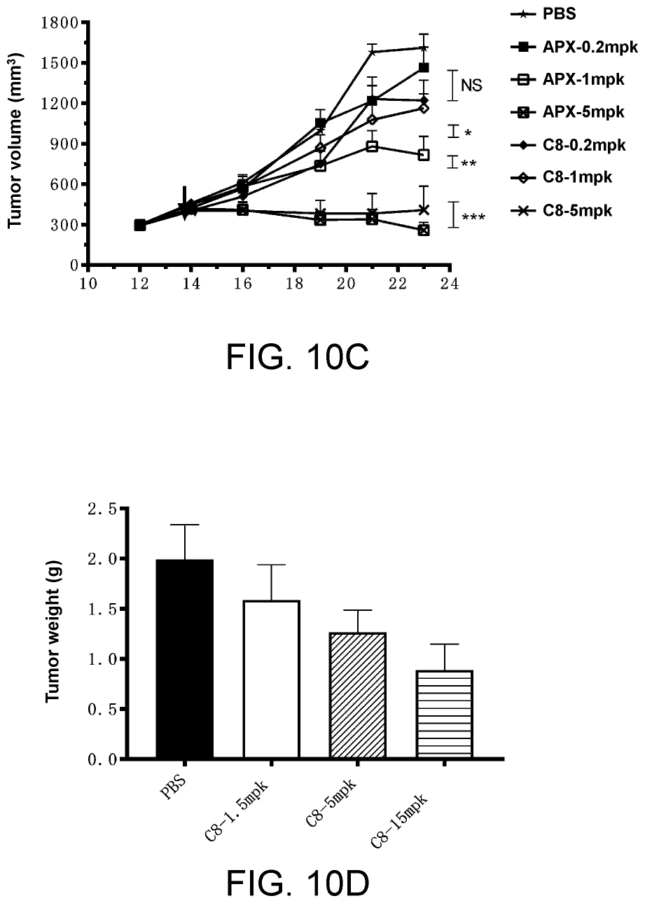

Six-week-old female nude mice (Vitalriver Balb/c Nude) were subcutaneously injected with 5 million human lymphoma Ramos cells on day 0 (starting from the day of inoculation of tumor cells, which was defined as day 0, and the next day was defined as day 1). When the average tumor volume reached 250 mm³, all mice (8 mice/group) were administered with the C8 anti-CD40 full-length antibody at 0.2 mg/kg, 1 mg/kg or 5 mg/kg (hereinafter, mg/kg is also abbreviated as mpk) by intraperitoneal injection three times a week (TIW) for two weeks. Tumor volume was measured prior to each injection, and tumor weight was measured two weeks after the first dosing. The final tumor volume and tumor weight were as shown in FIG. 10A and FIG. 10B, respectively, and the change in tumor volume was as shown in FIG. 10C.

The C8 anti-CD40 full-length antibody exhibited similar anti-tumor efficacy compared to the control antibody APX005M (also referred to herein as APX or APX005) (see US 20170246297, Apexigen Inc.).

The TGI (tumor growth inhibition rate) of the antibodies in each group was as shown in Table 4.

TABLE 4
TGI of APX005 and C8 anti-CD40 full-length antibody

| | Group | | | | | | |
|---|---|---|---|---|---|---|---|
| | | APX | APX | APX | C8 | C8 | C8 |
| Day | PBS | 0.2 mpk | 1 mpk | 5 mpk | 0.2 mpk | 1 mpk | 5 mpk |
| 14 | 0 | 8.02 | 8.06 | 8.84 | 12.08 | 2.53 | 12.58 |
| 16 | 0 | 7.14 | 4.75 | 32.8 | 16.61 | 5.73 | 33.78 |
| 19 | 0 | −5.45 | 26.17 | 66.32 | 24.84 | 12.81 | 61.47 |
| 21 | 0 | 22.88 | 44.29 | 78.42 | 22.10 | 31.70 | 75.70 |
| 23 | 0 | 9.18 | 49.25 | 83.82 | 24.24 | 27.75 | 74.59 |

### 3.2. Inhibition of Tumor Growth in Mouse Model of MC-38 Cells by Anti-CD40 Agonistic Antibody

MC38 is a mouse colon adenocarcinoma cell line that highly expresses mouse p53 protein (mp53) and does not express CD40 molecules.

The anti-tumor efficacy of the candidate anti-CD40 antibody was tested in a mouse subcutaneous transplantation model of MC-38 cells.

Six-week-old female mice (Vitalriver C57BL/6) were subcutaneously injected in the axilla with 1 million mouse colon adenocarcinoma cells (MC-38 cells) (National Infrastructure of Cell Line Resource, Cat. No. 3111C0001CCC000523). When the average tumor volume reached 80-100 mm³, all mice (8 mice/group) were administered with the C8 anti-CD40 antibody at 1.5 mg/kg, 5 mg/kg or 15 mg/kg (hereinafter, mg/kg is also abbreviated as mpk) by intraperitoneal injection three times a week (TIW) for three weeks. Tumor volume was measured prior to each injection, and tumor weight was measured three weeks after the first dosing. The final tumor weight was as shown in FIG. 10D.

It can be seen from FIG. 10D, the anti-CD40 agonistic antibody C8 molecule exhibits the anti-tumor efficacy at gradient concentrations compared to the negative control PBS.

### Example 4. Affinity Variant of C8 Clone Anti-CD40 Antibody

To obtain a change in the affinity of the C8 clone full-length antibody for a CD40 antigen, in this example, affinity variant design and functional determination were further performed on the C8 clone full-length antibody.

### 4.1 Construction of Affinity Variant Phage Display Library

Firstly, the C8-WT-VL and C8-WT-VH obtained in example 1.3 were subjected to AbM numbering to define CDRs and framework regions, and then a series of primers were designed to perform single site mutation or consecutive three-site mutations on the CDRs respectively, so as to construct the affinity variant phage display library.

### 4.2. Screening of Affinity Variant Phage Display Library

The affinity variant phage display library was screened by immunotube screening (i.e., solid phase screening). An immunotube was coated with antigen protein P17-His (prepared using a similar method in example 1), and the antigen-binding immunotube and the affinity variant phage display library were subjected to 2-3 rounds of panning comprising incubation, washing and elution, whereby a large number of the specific monoclonal antibodies against the antigen can be enriched. For the specific implementation method for screening the C8 affinity variant from the phage display library, reference can be made to the immunotube screening method in patent application number 202010236256.8.

After screening C8 affinity variants from the affinity variant phage display library, many candidate antibodies capable of binding to P17-His antigen with high specificity were obtained at ELISA level. According to the binding affinity of the Fab molecule, the first 11 candidate molecules having the highest binding affinity were selected for full-length antibody construction, in which the sequence of human IgG1 Fc silent (N297Q mutation) (i.e., huIgG1 (N297Q)) was linked to the heavy chain sequence of the Fab molecule to obtain a fully human IgG1 (N297Q) antibody with all light chains being k light chains.

### 4.3. Preparation of Full-Length Antibody of Affinity Variant Candidate Clone

See example 1.4 “expression, purification and concentration determination of full-length antibody” for specific methods. C8-1 antibody, C8-2 antibody, C8-3 antibody, C8-4 antibody, C8-5 antibody, C8-6 antibody, C8-7 antibody, C8-8 antibody, C8-9 antibody, C8-10 antibody and C8-11 antibody were obtained. The nucleotide and amino acid sequences of the variable regions of C8 parent antibody and each variant antibody were as shown in Table 3.

### 4.4. Determination of Non-Specific Binding of Affinity Variant Candidate Antibody

The relationship between the non-specific binding of the obtained affinity variant candidate antibody to different cells and the antibody concentrations used was initially detected. Both HEK293 cells and CHO-K cells are cells that do not naturally express CD40 molecules on the cell surface, and therefore antibodies that specifically bind to CD40 will not specifically bind to HEK293 cells and CHO-K cells. The positive control used in this experiment was F4AM4 antibody described in patent CN 202010825379.5, which was a fully humanized antibody with the light and heavy chain sequences as shown in F4AM4-LC and F4AM4-HC in Table 5, respectively.

In the experiment, HEK293 (ATCC: CRL-3216) cells and CHO-K (ATCC: CCL-61) cells were respectively seeded into a 96-well plate at 1.0×10⁵ cells/well. 100 μL of the anti-CD40 candidate antibody prepared in example 4.3 after gradient dilution was added to the 96-well plate. After incubation at 4° C. for 60 min, the cells were washed 5 times with a FACS buffer (PBS+5% FBS+2% BSA), added with 100 μL of PE-labeled anti-human IgG-Fc secondary antibody (Goat F(ab′)2 Anti-Human IgG-Fc (PE), pre-adsorbed, Abcam, Cat. No. ab98596), and then incubated at 4° C. for 30 min. The cells were then washed and tested by flow cytometry.

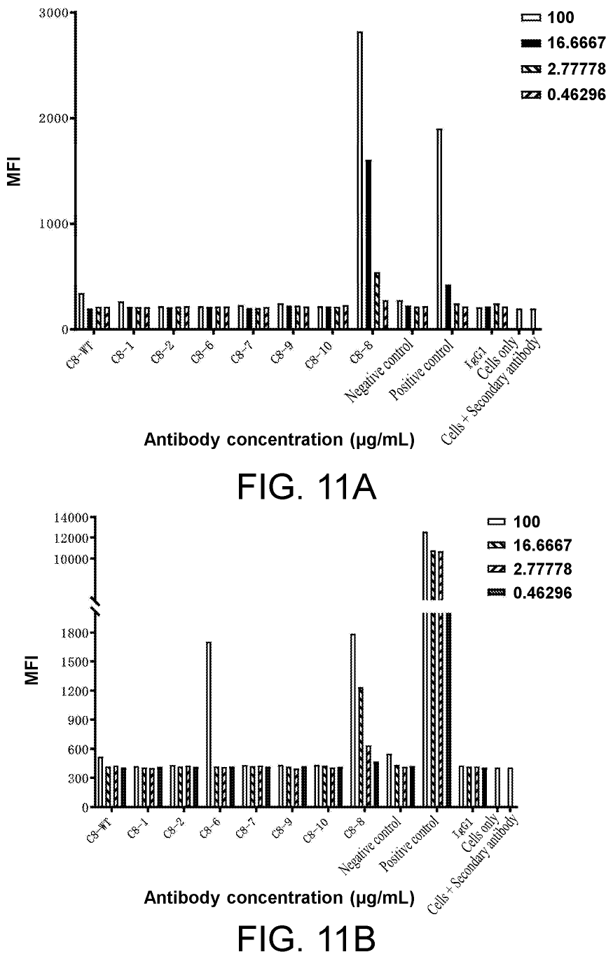

As shown in FIGS. 11A and 11B, C8-8 clone at a higher concentration exhibited the non-specific binding to both HEK293 and CHO-K cells; C8-6 clone at a high concentration (100 μg/mL) exhibited certain non-specific binding to HEK293, while other candidate clones and IgG1 had no non-specific binding to these two cell lines. Therefore, in the subsequent experiments, candidate molecules with no non-specific binding were used for the next test.

TABLE 5
Light and heavy chain sequences of
positive control antibody

| | |
|---|---|
| F4AM4- | DIQMTQSPSSLSASVGDRVTITCRASQDISNYLNWYQQKPGKA |
| LC | PKLLIYYTSRLHSGVPSRFSGSGSGTDYTLTISNLQPEDIATY |
| | YCQQGKNYPFTFGSGTKLEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 61) (wherein the light chain |
| | variable region sequence is bold) |
| F4AM4- | QVQLVQSGAEVKKPGASVKMSCKASGYTFTSSVMHWVRQAPGQ |
| HC | GLEWIGYINPYTDGTKYAQKFQGRATLTSDKSTSTAYMEFSSL |
| | RSEDTAVYYCGRPYYGTRYGSWFAYWGQGTLVTVSSASTKGPS |
| | VFPLAPSSKSTSGGTAALGCLVKDYFPEPVTVSWNSGALTSGV |
| | HTFPAVLQSSGLYSLSSVVTVPSSSLGTQTYICNVNHKPSNTK |
| | VDKKAEPKSCDKTHTCPPCPAPELLGGPSVFLFPPKPKDTLMI |
| | DRTPEVTCVVVDVSHEDPEVKFNWYVDGVEVHNAKTKPREEQY |
| | NSTYRVVSVLTVLHQDWLNGKEYKCKVSNKALPAPIEKTISKA |
| | KGQPREPQVYTLPPSRDELTKNQVSLTCLVKGFYPSDIAVEWE |
| | SNGQPENNYKTTPPVLDSDGSFFLYSKLTVDKSRWQQGNVFSC |
| | SVMHEALHNHYTQKSLSLSPGK (SEQ ID NO: 62) |
| | (wherein the heavy chain variable region |
| | sequence is bold) |

### 4.5. Cell-Based Functional Determination of Affinity Variant Candidate Antibody

### 4.5.1. CD40 Binding and Cross-Reactivity of Affinity Variant Candidate Antibody

To confirm the binding activity and cross-reactivity of the affinity variant antibody of example 4.3, artificially constructed CHO-K cells expressing full-length human CD40 (CHO-K-huCD40 cells) were seeded into a 96-well plate at 1.0×10⁵ cells/well. 100 μL of diluted anti-CD40 candidate antibody prepared in example 4.3 was added to the 96-well plate. After incubation at 4° C. for 30 min, the cells were washed 5 times with a FACS buffer (PBS+5% FBS+2% BSA), added with 100 μL of APC-labeled anti-human IgG secondary antibody, and then incubated at 4° C. for 30 min. The cells were then washed and tested by flow cytometry.

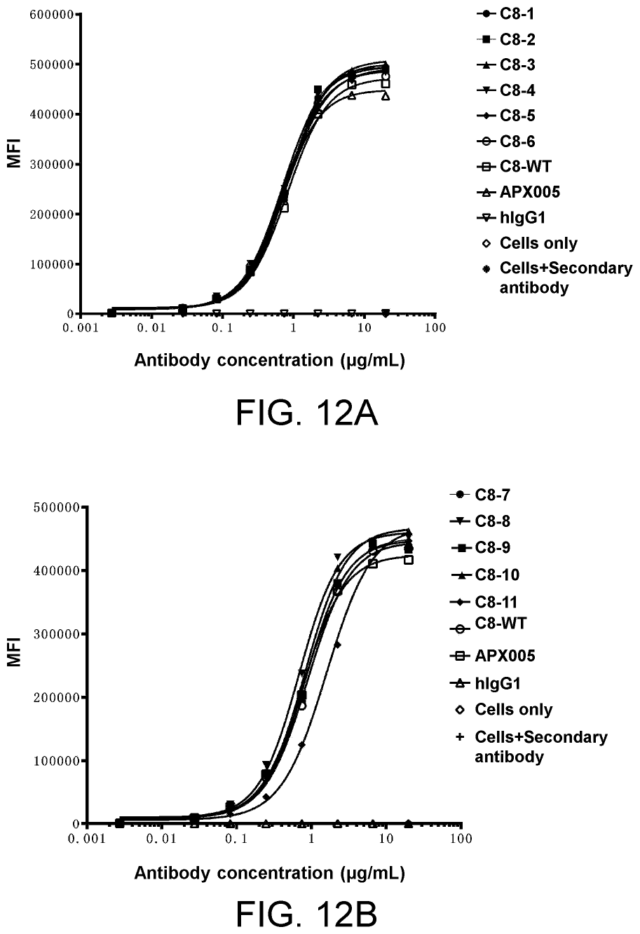

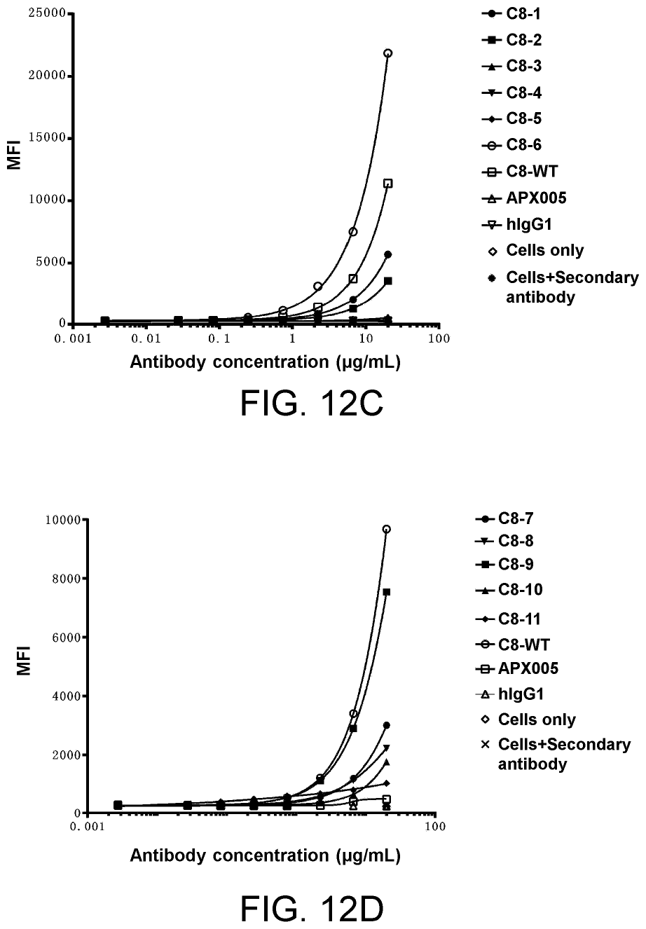

As shown in FIG. 12A and FIG. 12B, after the full-length antibody construction of affinity variant Fab, all candidate antibodies exhibited the affinity for human CD40, which was equivalent to that of the control antibody APX005 (since APX005 exhibited better efficacy on pancreatic cancer in clinical phase II results, the control antibody was set as APX005 in the relevant experiments for C8 antibody affinity variants; and the APX005 sequence was derived from Apexigen Inc. US 20120301488, prepared by Sanyou Biopharmaceuticals Co., Ltd.).

The cross-reactivity of the affinity variant candidate antibody prepared in example 4.3 to mouse CD40 was tested using artificially constructed CHO-K cells expressing full-length mouse CD40 (Uniprot #P27512) (CHO-K-mouseCD40 cells). The CHO-K cells expressing mouse CD40 were seeded into a 96-well plate at 1.0×10⁵ cells/well. 100 μL of diluted affinity variant candidate antibody and the control antibody (APX005) were added to the 96-well plate, respectively. After incubation at 4° C. for 30 minutes, the cells were washed, added with FITC-labeled anti-human IgG Fcγ (AffiniPure F(ab′)₂ Fragment Goat Anti-Human IgG, Fcγ fragment specific, Jackson Immunoresearch, Cat. No. 109-006-098), and incubated for 30 minutes at 4° C. The cells were then washed and tested by flow cytometry.

As shown in FIG. 12C and FIG. 12D, C8-6 exhibited a better binding activity to mouse CD40 antigen than female parent C8-WT. C8-1, C8-2, C8-7, C8-8, C8-9, C8-10 and C8-11 all exhibited the binding activity to mouse CD40 antigen.

4.5.2. Affinity Variant Candidate Antibody-Mediated Induction of CD95 in Ramos Cells (with Crosslinking Agent, i.e., Presence of Crosslinking Effect)

To test whether the affinity variant candidate antibody binds to CD40 on Ramos cells and activates the downstream signaling pathway of CD40 in the presence of a crosslinking agent, Ramos cells (ATCC No.: CRL-1596) were plated into wells containing a complete RPMI 1640 medium at 1.0×10⁵ cells/well. To the Ramos cells were added diluted affinity variant candidate antibodies, control antibodies APX005 and CD40L (20 μg/mL for the first well, 3-fold gradient dilution, 8 concentration wells) and a crosslinking agent (AffiniPure F(ab′)₂ Fragment Goat Anti-Human IgG, Fcγ fragment specific, Jackson Immunoresearch, Cat. No. 109-006-098) at a concentration of 20 μg/mL, and the mixture was incubated overnight at 37° C. The CD95 expression was determined by flow cytometry.

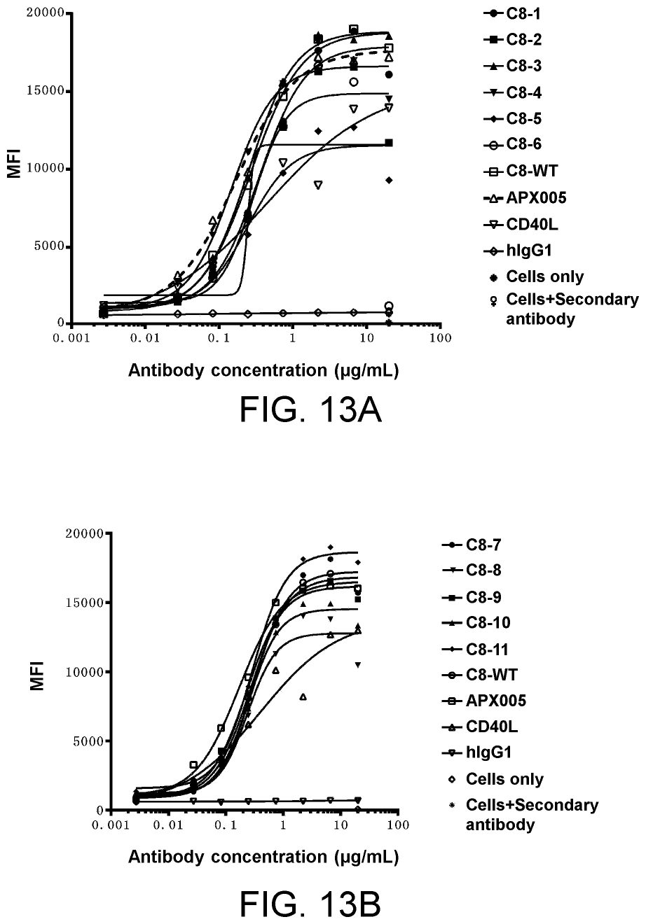

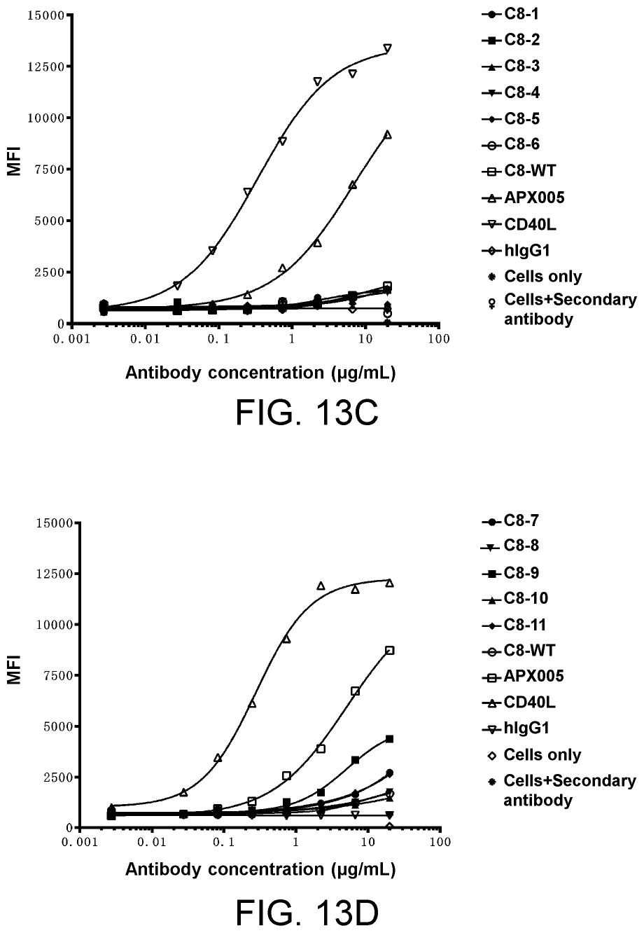

As shown in FIG. 13A and FIG. 13B, all affinity variant antibodies exhibited CD95-induced activity comparable to that of APX005, with no significant difference.

4.5.3. Affinity Variant Candidate Antibody-Mediated Induction of CD95 in Ramos Cells (without Crosslinking Agent, i.e., Absence of Crosslinking Effect)

To test whether the affinity variant candidate antibody binds to CD40 on Ramos cells and activates the downstream signaling pathway of CD40 in the absence of a crosslinking agent, Ramos cells (ATCC No.: CRL-1596) were plated into wells containing a complete RPMI 1640 medium at 1.0×10⁵ cells/well. Diluted affinity variant candidate antibodies and control antibodies APX005 and CD40L (20 μg/mL for the first well, 3-fold gradient dilution, 8 concentration wells) were added to the Ramos cells, and the mixture was incubated overnight at 37° C. The CD95 expression was determined by flow cytometry.

As shown in FIG. 13C and FIG. 13D, all affinity variant antibodies exhibited weaker CD95-induced activity than APX005 and CD40L, wherein C8-2, C8-6, C8-9, C8-10 and C8-11 exhibited about 10 times weaker CD95-induced activity than APX005. This indicates that the candidate molecule of the present invention, in the absence of a crosslinking agent, shows significantly lower ability to activate CD40 molecules compared to APX005, and can be expected to weakly agonize the CD40 molecules while having significantly less toxic and side effects compared to APX005. At present, the major adverse event of anti-CD40 agonistic antibodies in clinical trials is hepatotoxicity, which is due to a large number of FcR receptors expressed on the surface of liver cells. Furthermore, agonistic antibodies used in clinical trials (such as APX005) often have normal Fc function and are able to form a crosslinking effect with FcR receptors on the surface of liver cells, thereby non-specifically activating CD40-expressing immune cells on the surface of liver cells, resulting in hepatotoxicity with a lower therapeutic index. By analyzing the mechanism of a crosslinking effect, it can be found that the crosslinking effect causing hepatotoxicity is due to the binding to Fc receptors, and if the crosslinking effect generated by Fc receptors can be controlled to significantly enhance the activation effect at lesion sites, the hepatotoxicity can be controlled and the therapeutic index can be increased, which puts forward a high requirement for the difference in agonistic activity of antibody drug molecules in the presence or absence of a crosslinking effect.

To maximize the ratio of killing efficacy to toxicity, it is desired in the present invention to increase the therapeutic index by expanding the maximum difference degree of an agonistic effect between the presence and absence of a crosslinking effect, which means significantly generating an immune activation reaction in the presence of a crosslinking effect, and not generating an immune activation reaction or only generating a weak immune activation reaction in the absence of a crosslinking effect. It can be seen from this example that, with respect to APX005 molecules, the difference in the immune cell agonistic activity between the presence and absence of a crosslinking agent is less, that is, the ratio of efficacy to toxicity of the final drug is small. The candidate antibodies of the present invention are capable of maximizing the ratio of efficacy to toxicity in the presence or absence of a crosslinking agent, maximizing the killing effect of activated immune cells within tumor microenvironment, while avoiding toxic and side effects such as hepatotoxicity caused by non-specific activation of immune cells at non-tumor sites. If the candidate antibodies of the present invention are used to construct a diabody, this feature can be used to achieve the specific high-intensity activation of immune cells at lesion sites and achieve the low agonistic activity at sites such as hepatocytes where a crosslinking effect cannot be formed, thereby achieving the objective of specifically killing tumor cells without causing adverse events such as hepatotoxicity, effectively increasing therapeutic index and improving safety.

### 4.5.4. Preparation of Jurkat NF-κB Luciferase Reporter Gene Stably Transformed Cell Line

Firstly, a vector pGL4.32[Luc2p/NF-κB-RE/Hygro] plasmid (Promega, Cat. No. E8491) was transformed into Jurkat cells (ATCC® TIB-152™) via electrotransformation by an electroporator (Invitrogen, Neon™ Transfection System, MP922947). After electrotransformation, the obtained cells were separately transferred to a RPMI 1640 medium (Hyclone, SH30243.01) containing 10% FBS (Gibco, 15140-141) without antibiotics, seeded into a 6-well plate cell culture dish and cultured for 48 h, and then subpackaged into a 96-well cell culture plate at an average density of 1500 cells/well. Hygromycin B (BasalMedia, S160J7) with a final concentration of 500 μg/mL was added for screening. The growth of cell clones was observed after about 2-3 weeks, and cell lines forming clones were picked and transferred to a 24-well plate. After cell scale-up culture, some clones were transferred to a 96-well white bottom plate (Corning, 3610), stimulated with phorbol ester (using a concentration of 10 ng/ml) and ionomycin (using a concentration of 1 nM), cultured in a 5% CO₂ incubator at 37° C. for 6 h, and then added with Bright-Lite substrates (Vazyme, DD1204-03). The expression levels of NF-κB in different clones were evaluated after the signal values were read by a microplate reader (Molecular Devices: Spectramax i3x), thereby obtaining the Jurkat cell line that highly expresses NF-κB gene. The Jurkat NF-κB luciferase reporter gene stably transformed cell clone that highly expresses NF-κB was cryopreserved for later use.

### 4.5.5. Construction of Jurkat CD40/NF-κB Luciferase Reporter Gene Cell Line

In this example, a Jurkat CD40/NF-κB luciferase reporter gene cell line was constructed for screening candidate antibody molecules capable of activating downstream signaling activity of CD40. Monoclonal cell lines were screened using the Jurkat NF-κB luciferase reporter gene stably transformed cell line prepared in example 4.5.4, on the basis of which the full-length expression gene sequence of CD40 (Uniprot Gene ID: P25942) was stably transformed. CD40L recombinant protein was added to this cell line culture system to activate the transcription and expression of intracellular NF-κB luciferase reporter gene by binding to CD40, and a catalytic substrate for luciferase was added to generate a fluorescence signal. The Jurkat CD40/NF-κB luciferase reporter gene cell line was prepared as follows.

Construction of a plasmid expressing full-length human CD40 (Met1-Gln277): DNA fragments containing a humanized CD40 protein were synthesized by gene synthesis technology and cloned into a pLVX-Puro expression vector (Clontech, 632164). E. coli was introduced by chemical transformation. E. coli single clones were picked and sequenced to obtain correct plasmid clones, and the plasmid was extracted and sequenced again for confirmation. Electrotransformation: The Jurkat cells prepared in example 4.5.4 were cultured using a RPMI 1640 serum-free medium (Gibco, Cat. No. 11875085). The cells were passaged to 2×10⁵/mL one day before electrotransformation, and the constructed plasmid was transformed into the Jurkat NF-κB luciferase reporter gene stably transformed cell line using an electrotransformation kit (Cat. No. MPK10096) and an electroporator (Cat. No. MP922947) on the next day. The cells after electrotransformation were transferred to a RPMI 1640 medium, and cultured in a 37° C. incubator for 48 h. Cell plating after electrotransformation: The Jurkat cells after electrotransformation were plated in a 96-well plate at 1000 cells/well, added with puromycin at a final concentration of 2 μg/mL, cultured in a 37° C. carbon dioxide incubator for 14 days, and then supplemented with a RPMI 1640 medium containing 2 μg/mL puromycin. Clone picking, cell scale-up culture and FACS identification: Single cell clones growing in the 96-well plate were picked and transferred to a 24-well plate for further scale-up culture. After that, the cell line with successful stable transformation of human CD40 was identified by FACS.

4.5.6. Affinity Variant Candidate Antibody for Activating Downstream NF-κB Luciferase Reporter Gene Signaling Activity of CD40 (with Crosslinking Agent, i.e., Presence of Crosslinking Effect)

In this example, to test whether the affinity variant candidate antibody has the function of activating CD40 signaling pathway in the presence of a crosslinking agent, the Jurkat CD40/NF-κB luciferase reporter gene cell line prepared in example 4.5.5 was used as a material to detect the ability of the candidate molecule to bind to CD40 and thereby activate the downstream NF-κB luciferase reporter gene expression. The specific implementation method was as follows.

Female parent C8 molecule, affinity variant candidate antibodies and control antibody APX005 were gradiently diluted using a RPMI 1640 medium (10 μg/mL for the first well, 3-fold gradient dilution, 8 concentration wells), and added with a 10 μg/mL crosslinking agent (AffiniPure F(ab′)₂ Fragment Goat Anti-Human IgG, Fcγ fragment specific, Jackson Immunoresearch, Cat. No. 109-006-098) for pre-mixing. The premix liquid of the antibodies and crosslinking agent was added to a 96-well plate at 50 μL per well. The Jurkat CD40/NF-κB luciferase reporter gene cell line was resuscitated, and the cells passaged 2-4 times in good growth conditions were used in the experiment. The cells were washed and resuspended with a RPMI 1640 medium, counted, adjusted to achieve a cell density of 2×10⁶ cells/mL, added at 50 μL per well to the 96-well cell culture plate containing the premix liquid of the antibodies and crosslinking agent, and then incubated in a 37° C. incubator at for 6 h. After culture, 30 L of luciferase substrate Bright-Lite (Vazyme, DD1204-03) was added to each well, and the fluorescence value of each well of the 96-well plate was detected after shaking for 5 min.

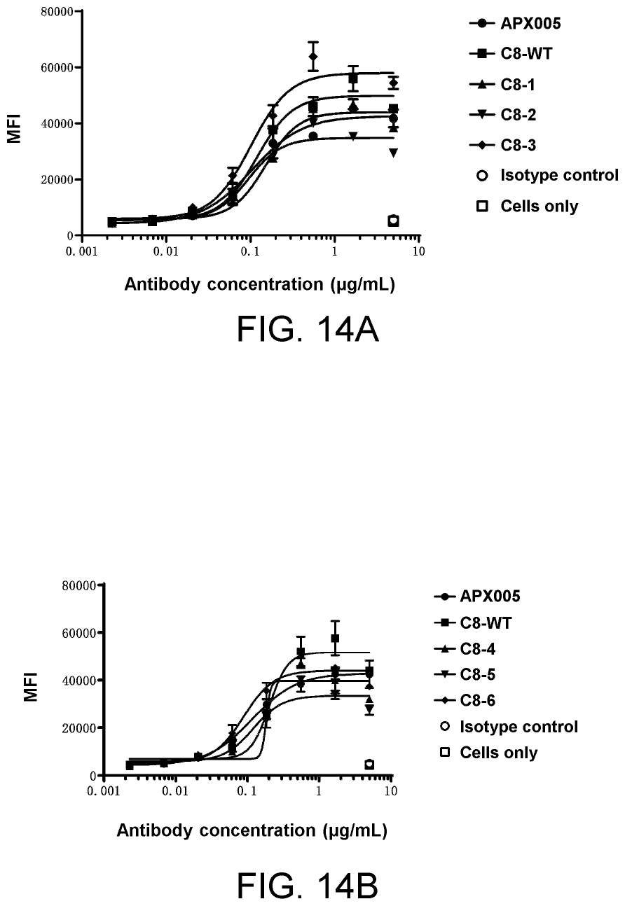

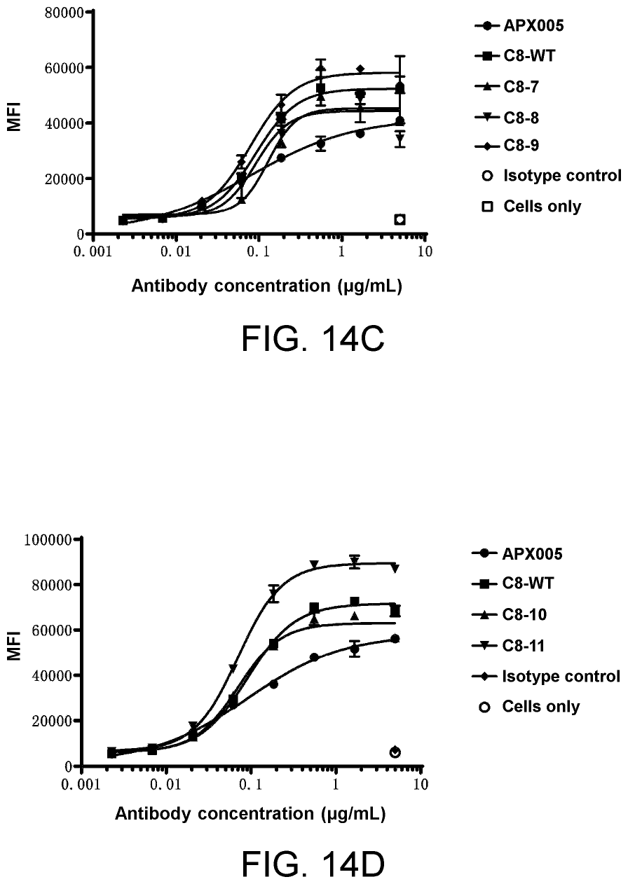

As shown in FIG. 14A, FIG. 14B, FIG. 14C and FIG. 14D, in the presence of a crosslinking agent, the female parent C8 molecules and all affinity variant antibodies exhibited comparable activation of downstream NF-κB luciferase reporter gene signaling activity of CD40 to APX005, with no significant difference.

4.5.7. Affinity Variant Candidate Antibody for Activating Downstream NF-κB Luciferase Reporter Gene Signaling Activity of CD40 (without Crosslinking Agent, i.e., Absence of Crosslinking Effect)

In this example, to test whether the affinity variant candidate antibody has the function of activating CD40 signaling pathway in the absence of a crosslinking agent, a Jurkat CD40/NF-κB luciferase reporter gene cell line was used as a material to detect the ability of the candidate molecule to bind to CD40 and thereby activate the downstream NF-κB luciferase reporter gene expression. The specific implementation method was as follows.

Female parent C8 molecule, various affinity variant candidate antibodies, and control antibody APX005 were gradiently diluted using a RPMI 1640 medium (10 μg/mL for the first well, 3-fold gradient dilution, 8 concentration wells), and the diluted candidate antibodies and control antibody were added to a 96-well plate at 50 μL per well. The Jurkat CD40/NF-κB luciferase reporter gene cell line was resuscitated, and the cells passaged 2-4 times in good growth conditions were used in the experiment. The cells were washed and resuspended with a RPMI 1640 medium, counted, adjusted to achieve a cell density of 2×10⁶ cells/mL, added at 50 μL per well to the 96-well cell culture plate containing the antibodies, and then incubated in a 37° C. incubator at for 6 h. After culture, 30 μL of luciferase substrate Bright-Lite (Vazyme, DD1204-03) was added to each well, and the fluorescence value of each well of the 96-well plate was detected after shaking for 5 min.

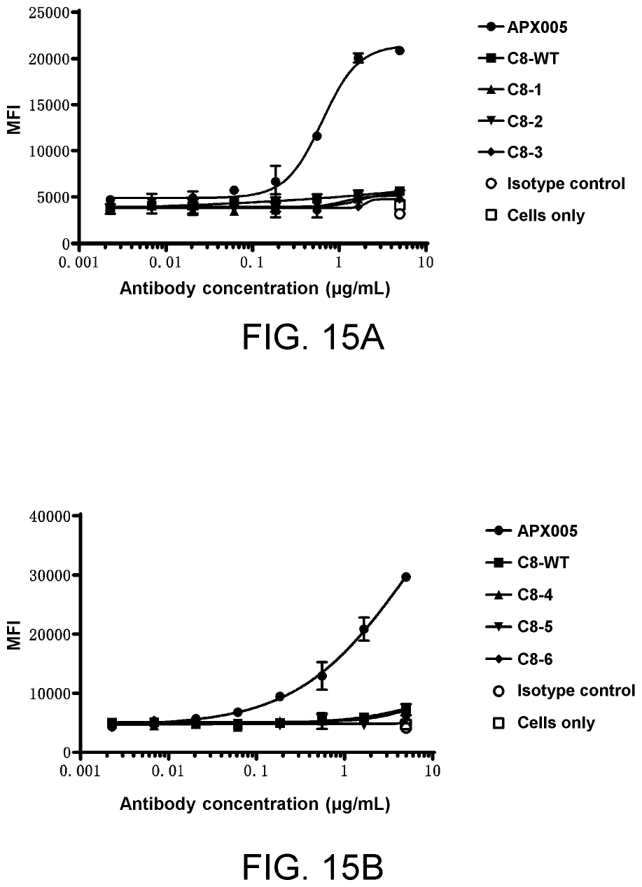

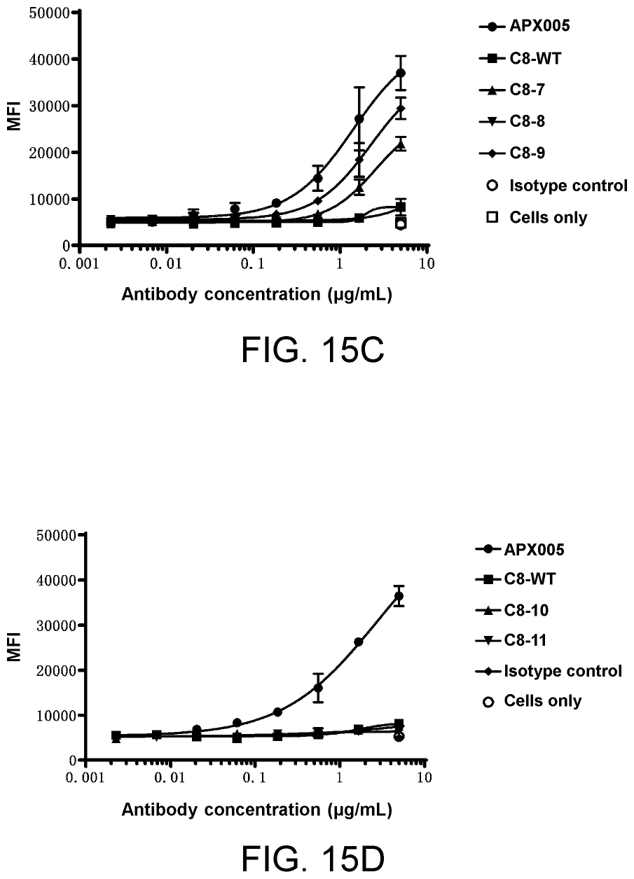

As shown in FIG. 15A, FIG. 15B, FIG. 15C and FIG. 15D, in the absence of a crosslinking agent, APX005 exhibited strong activation of downstream NF-κB luciferase reporter gene signaling activity of CD40. Except for C8-7 and C8-9, which exhibited certain activating ability, the affinity mutant candidate antibodies had no activating ability, and it can be expected to have significantly less toxic and side effects than APX005 while weakly activating CD40 molecules. From the comparison of the activation of the CD40 signaling pathway by various candidate molecules in FIG. 14A-FIG. 14D and FIG. 15A-FIG. 15D, in the presence or absence of a crosslinking effect, the activation of the CD40 signaling pathway by female parent C8 molecules and various affinity variant candidate molecules was basically identical to the B cell activation in FIG. 13A-FIG. 13D, which also verified the results of B cell (Ramos cell) activation test from the molecular mechanism. It can be seen from the test results that B cell activation caused by the anti-CD40 antibody of the present invention was formed by activating the CD40 signaling pathway.

### 4.6. Detection of Affinity and Kinetics of Affinity Variant Candidate Antibodies

In this example, the affinity of C8 parent antibody (C8-WT) and affinity variant antibody for human CD40 antigen (P17-His) was detected using an Fortebio Octet RED96 instrument.

Material preparation: 1 g of BSA and 500 μL of Tween 20 were added to 1000 mL of 1×PBS and mixed uniformly. The mixture was filtered, and then subpackaged and stored. 0.1 mL of glycine solution (0.1 M, pH 2.0) was pipetted, 0.9 mL of ultrapure water was added, and the mixture was mixed uniformly. The antibodies were diluted to 10 μg/mL with a KB buffer, and the antigens were diluted with a KB buffer to serial concentration gradients, i.e., 40 nM, 20 nM, 10 nM, 5 nM and 0 nM.

Experiment procedure: The sample plate (GreinierBio, PN655209) was tested after a sensor (Protein A sensor) was pre-wetted in the dark for at least 10 min, and after confirming the accuracy, the test was performed according to the preset programs. KB buffer was added at 200 μL/well to the wells of columns 1, 10 and 12 of sample plate 1; the glycine solution (0.01 M, pH 2.0) was added to the wells of column 11; the prepared sample solution was added to the wells of columns 2-8 (5 replicate wells for one sample); and P17-His was sequentially added to the wells of column 9 in a descending order of the concentration. The data results were detailed in Table 6.

TABLE 6
Detection results of affinity of C8 parent antibody
(C8-WT) and affinity variant antibodies

| Antibody name | KD (M) | ka (1/Ms) | kd (1/s) | R² |
|---|---|---|---|---|
| APX005 | 6.30E−09 | 1.98E+06 | 1.24E−02 | 0.973 |
| C8-WT | 1.93E−08 | 8.53E+05 | 1.64E−02 | 0.974 |
| C8-1 | 1.27E−08 | 1.16E+06 | 1.48E−02 | 0.969 |
| C8-2 | 1.19E−08 | 9.32E+05 | 1.11E−02 | 0.968 |
| C8-3 | 4.51E−09 | 2.46E+06 | 1.11E−02 | 0.972 |
| C8-4 | 5.34E−09 | 1.36E+06 | 7.25E−03 | 0.969 |
| C8-5 | 8.23E−09 | 9.94E+05 | 8.18E−03 | 0.982 |
| C8-6 | 9.04E−09 | 1.09E+06 | 9.84E−03 | 0.99 |
| C8-7 | 1.24E−08 | 8.95E+05 | 1.11E−02 | 0.99 |
| C8-8 | 1.03E−08 | 1.07E+06 | 1.10E−02 | 0.982 |
| C8-9 | 9.27E−09 | 1.21E+06 | 1.12E−02 | 0.987 |
| C8-10 | 6.82E−09 | 1.24E+06 | 8.47E−03 | 0.984 |
| C8-11 | 9.24E−09 | 1.60E+06 | 1.48E−02 | 0.976 |

It can be seen from Table 6 that the C8 parent antibody (C8-WT) and the affinity variant antibodies bind to human CD40 antigen with an affinity of about 0.5×10⁻⁸ M to 2×10⁻⁸ M, wherein all affinity variant antibodies have an improved affinity compared to the C8 parent antibody (C8-WT) and are affinity matured antibodies.

1. An anti-CD40 antibody or an antigen-binding fragment, comprising a light chain variable region and a heavy chain variable region, the light chain variable region comprising three complementarity determining regions, i.e., LCDR1, LCDR2 and LCDR3, respectively, and the heavy chain variable region comprising three complementarity determining regions, i.e., HCDR1, HCDR2 and HCDR3, respectively, wherein:
(a) LCDR1 comprises an amino acid sequence as shown in SEQ ID NO: 1 or a variant of SEQ ID NO: 1 having no more than 2 or no more than 1 amino acid change;
(b) LCDR2 comprises an amino acid sequence selected from any one of amino acid sequences of SEQ ID NOs: 2, 5, 6 and 7, or a variant of any one of amino acid sequences of SEQ ID NOs: 2, 5, 6 and 7 having no more than 2 or no more than 1 amino acid change;
(c) LCDR3 comprises an amino acid sequence selected from any one of amino acid sequences of SEQ ID NOs: 3 and 4, or a variant of any one of amino acid sequences of SEQ ID NOs: 3 and 4 having no more than 2 or no more than 1 amino acid change;
(d) HCDR1 comprises an amino acid sequence selected from any one of amino acid sequences of SEQ ID NOs: 8, 12, 14 and 20, or a variant of any one of amino acid sequences of SEQ ID NOs: 8, 12, 14 and 20 having no more than 2 or no more than 1 amino acid change;
(e) HCDR2 comprises an amino acid sequence selected from any one of amino acid sequences of SEQ ID NOs: 9 and 21, or a variant of any one of amino acid sequences of SEQ ID NOs: 9 and 21 having no more than 2 or no more than 1 amino acid change;
and (f) HCDR3 comprises an amino acid sequence selected from any one of amino acid sequences of SEQ ID NOs: 10, 11, 13, 15, 16, 17, 18 and 19, or a variant of any one of amino acid sequences of SEQ ID NOs: 10, 11, 13, 15, 16, 17, 18 and 19 having no more than 2 or no more than 1 amino acid change,
wherein the amino acid change is an amino acid addition, deletion or substitution.

2. The anti-CD40 antibody or the antigen-binding fragment according to claim 1, comprising:
(i) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 10 or SEQ ID NO: 10 having no more than 2 or no more than 1 amino acid change;
(ii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 4 or SEQ ID NO: 4 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 10 or SEQ ID NO: 10 having no more than 2 or no more than 1 amino acid change;
(iii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 11 or SEQ ID NO: 11 having no more than 2 or no more than 1 amino acid change;
(iv) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 5 or SEQ ID NO: 5 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 12 or SEQ ID NO: 12 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 10 or SEQ ID NO: 10 having no more than 2 or no more than 1 amino acid change;
(v) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 13 or SEQ ID NO: 13 having no more than 2 or no more than 1 amino acid change;
(vi) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 6 or SEQ ID NO: 6 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 14 or SEQ ID NO: 14 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 10 or SEQ ID NO: 10 having no more than 2 or no more than 1 amino acid change;
(vii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 15 or SEQ ID NO: 15 having no more than 2 or no more than 1 amino acid change;
(viii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 16 or SEQ ID NO: 16 having no more than 2 or no more than 1 amino acid change;
(ix) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 17 or SEQ ID NO: 17 having no more than 2 or no more than 1 amino acid change;
(x) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 18 or SEQ ID NO: 18 having no more than 2 or no more than 1 amino acid change;
(xi) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 2 or SEQ ID NO: 2 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 8 or SEQ ID NO: 8 having no more than 2 or no more than 1 amino acid change, HCDR2 comprising an amino acid sequence of SEQ ID NO: 9 or SEQ ID NO: 9 having no more than 2 or no more than 1 amino acid change, and HCDR3 comprising an amino acid sequence of SEQ ID NO: 19 or SEQ ID NO: 19 having no more than 2 or no more than 1 amino acid change; or
(xii) LCDR1 comprising an amino acid sequence of SEQ ID NO: 1 or SEQ ID NO: 1 having no more than 2 or no more than 1 amino acid change, LCDR2 comprising an amino acid sequence of SEQ ID NO: 7 or SEQ ID NO: 7 having no more than 2 or no more than 1 amino acid change, LCDR3 comprising an amino acid sequence of SEQ ID NO: 3 or SEQ ID NO: 3 having no more than 2 or no more than 1 amino acid change, HCDR1 comprising an amino acid sequence of SEQ ID NO: 20 or SEQ ID NO: 20 having no more than 2 or no more than 1 amino acid change, HCDR2 comprising an amino acid sequence of SEQ ID NO: 21 or SEQ ID NO: 21 having no more than 2 or no more than 1 amino acid change, and LCDR3 comprising an amino acid sequence of SEQ ID NO: 10 or SEQ ID NO: 10 having no more than 2 or no more than 1 amino acid change.

3. The anti-CD40 antibody or the antigen-binding fragment according to claim 1, comprising:
LCDR1 comprising an amino acid sequence RSSQGIRSSLA (SEQ ID NO: 1), LCDR2 comprising an amino acid sequence GX₁SSLX₂X₃ (SEQ ID NO: 56), and LCDR3 comprising an amino acid sequence QQLX₄SFPST (SEQ ID NO: 57), wherein X₁ is A or G, X₂ is E or L, X₃ is G or V, and X₄ is N or A; and
HCDR1 comprising an amino acid sequence GFTX₅GSYEMX₆ (SEQ ID NO: 58), HCDR2 comprising an amino acid sequence YISSX₇GETTD (SEQ ID NO: 59), and HCDR3 comprising an amino acid sequence DVFFFX₈X₉SX₁₀X₁₁X₁₂X₁₃ AYGMDV (SEQ ID NO: 60), wherein X₅ is F, A or P, X₆ is N or D, X₇ is S or A, X₈ is D or S, X₉ is S or P, X₁₀ is G or R, X₁₁ is D, P, S or F, X₁₂ is P, Nor R, and X₁₃ is G or H;
wherein the anti-CD40 antibody or the antigen-binding fragment comprises:
(i) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 10;
(ii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 4, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 10;
(iii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 11;
(iv) LCDR1 comprising an amino acid sequence of SEQ ID NO: 1, LCDR2 comprising an amino acid sequence of SEQ ID NO: 5, LCDR3 comprising an amino acid sequence of SEQ ID NO: 3, HCDR1 comprising an amino acid sequence of SEQ ID NO: 12, HCDR2 comprising an amino acid sequence of SEQ ID NO: 9, and HCDR3 comprising an amino acid sequence of SEQ ID NO: 10;
(v) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 13;
(vi) LCDR1 comprising an amino acid sequence of SEQ ID NO: 1, LCDR2 comprising an amino acid sequence of SEQ ID NO: 6, LCDR3 comprising an amino acid sequence of SEQ ID NO: 3, HCDR1 comprising an amino acid sequence of SEQ ID NO: 14, HCDR2 comprising an amino acid sequence of SEQ ID NO: 9, and HCDR3 comprising an amino acid sequence of SEQ ID NO: 10;
(vii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 15;
(viii) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 16;
(ix) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 17;
(x) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 18;
(xi) LCDR1 comprising an amino acid sequence of SEQ ID NO: 1, LCDR2 comprising an amino acid sequence of SEQ ID NO: 2, LCDR3 comprising an amino acid sequence of SEQ ID NO: 3, HCDR1 comprising an amino acid sequence of SEQ ID NO: 8, HCDR2 comprising an amino acid sequence of SEQ ID NO: 9, and HCDR3 comprising an amino acid sequence of SEQ ID NO: 19; or
(xii) LCDR1 comprising an amino acid sequence of SEQ ID NO: 1, LCDR2 comprising an amino acid sequence of SEQ ID NO: 7, LCDR3 comprising an amino acid sequence of SEQ ID NO: 3, HCDR1 comprising an amino acid sequence of SEQ ID NO: 20, HCDR2 comprising an amino acid sequence of SEQ ID NO: 21, and HCDR3 comprising an amino acid sequence of SEQ ID NO: 10.

4. The anti-CD40 antibody or the antigen-binding fragment according to claim 1, comprising a light chain variable region and a heavy chain variable region, wherein:
(i) the light chain variable region comprises any one of amino acid sequences of SEQ ID NOs: 23, 29, 33, 39 and 53, and the heavy chain variable region comprises any one of amino acid sequences of SEQ ID NOs: 25, 31, 35, 37, 41, 43, 45, 47, 49, 51 and 55; or
(ii) the light chain variable region comprises an amino acid sequence having at least 85%, 90%, 91%, 92%, 93%, 94%, 95%, 96%, 97%, 98% or 99% identity to any one of amino acid sequences of SEQ ID NOs: 23, 29, 33, 39 and 53, and the heavy chain variable region comprises an amino acid sequence having at least 85%, 90%, 91%, 92%, 93%, 94%, 95%, 96%, 97%, 98% or 99% identity to any one of amino acid sequences of SEQ ID NOs: 25, 31, 35, 37, 41, 43, 45, 47, 49, 51 and 55;
wherein the anti-CD40 antibody or the antigen-binding fragment comprises:
(i) a light chain variable region comprising an amino acid sequence of SEQ ID NO: 23 and a heavy chain variable region comprising an amino acid sequence of SEQ ID NO: 25;
(ii) a light chain variable region comprising an amino acid sequence of SEQ ID NO: 29 and a heavy chain variable region comprising an amino acid sequence of SEQ ID NO: 25;
(iii) a light chain variable region comprising an amino acid sequence of SEQ ID NO: 23 and a heavy chain variable region comprising an amino acid sequence of SEQ ID NO: 31;
(iv) a light chain variable region comprising an amino acid sequence of SEQ ID NO: 33 and a heavy chain variable region comprising an amino acid sequence of SEQ ID NO: 35;
(v) a light chain variable region comprising an amino acid sequence of SEQ ID NO: 23 and a heavy chain variable region comprising an amino acid sequence of SEQ ID NO: 37;
(vi) a light chain variable region comprising an amino acid sequence of SEQ ID NO: 39 and a heavy chain variable region comprising an amino acid sequence of SEQ ID NO: 41;
(vii) a light chain variable region comprising an amino acid sequence of SEQ ID NO: 23 and a heavy chain variable region comprising an amino acid sequence of SEQ ID NO: 43;
(viii) a light chain variable region comprising an amino acid sequence of SEQ ID NO: 23 and a heavy chain variable region comprising an amino acid sequence of SEQ ID NO: 45;
(ix) a light chain variable region comprising an amino acid sequence of SEQ ID NO: 23 and a heavy chain variable region comprising an amino acid sequence of SEQ ID NO: 47;
(x) a light chain variable region comprising an amino acid sequence of SEQ ID NO: 23 and a heavy chain variable region comprising an amino acid sequence of SEQ ID NO: 49;
(xi) a light chain variable region comprising an amino acid sequence of SEQ ID NO: 23 and a heavy chain variable region comprising an amino acid sequence of SEQ ID NO: 51; or
(xii) a light chain variable region comprising an amino acid sequence of SEQ ID NO: 53 and a heavy chain variable region comprising an amino acid sequence of SEQ ID NO: 55,
wherein the anti-CD40 antibody or the antigen-binding fragment is a fully human antibody.

5. The anti-CD40 antibody or the antigen-binding fragment according to claim 1, wherein the anti-CD40 antibody or the antigen-binding fragment is an IgG1, IgG2, IgG3, or IgG4 antibody.

6. The anti-CD40 antibody or the antigen-binding fragment according to claim 1, wherein the antigen-binding fragment is Fab, Fab′, F(ab′)2, Fv, single chain Fv, single chain Fab and diabody.

7. The anti-CD40 antibody or the antigen-binding fragment according to claim 1, having one or more of the following properties:
(1) binding to and activating CD40, wherein the CD40 is human CD40, cynomolgus monkey CD40 and mouse CD40, with a high affinity, wherein the high affinity is binding to CD40 with a Kd of 10⁻⁷ M to 10⁻¹⁰ M, as measured by ForteBio kinetic binding assay;
(2) enhancing a binding of CD40 to CD40L;
(3) activating an antigen presenting cell, including a dendritic cell (DC), a B cell, a monocyte and a macrophage, by binding to CD40 expressed on the antigen presenting cell;
(4) inducing a CD40-expressing B cell to express CD95;
(5) enhancing a B cell-mediated immune response;
(6) significantly enhancing a B cell-mediated immune response when a crosslinking effect is formed;
and (7) barely or weakly enhancing a B cell-mediated immune response when a crosslinking effect is not formed.

8. The anti-CD40 antibody or the antigen-binding fragment according to claim 1, wherein a glycosylation site in a CH2 domain of an immunoglobulin Fc region is eliminated, wherein the glycosylation site is eliminated by mutating N297 residue in a CH2 domain of a human IgG Fc region.

9. An isolated nucleic acid, encoding the anti-CD40 antibody or the antigen-binding fragment according to claim 1.

10. A vector containing the nucleic acid according to claim 9, wherein the vector is an expression vector.

11. A host cell containing the nucleic acid according to claim 9, wherein the host cell is selected from an E. coli cell, a yeast cell, a mammalian cell or other cells suitable for the preparation of an antibody or an antigen-binding fragment thereof.

12. A method for preparing the anti-CD40 antibody or the antigen binding fragment according to claim 1, comprising: culturing a host cell under conditions suitable for the expression of a nucleic acid encoding the anti-CD40 antibody or the antigen binding fragment, and optionally isolating the anti-CD40 antibody or the antigen binding fragment, and optionally the method further comprising: recovering the anti-CD40 antibody or the antigen binding fragment from the host cell, wherein the host cell is selected from an E. coli cell, a yeast cell, a mammalian cell or other cells suitable for the preparation of an antibody or an antigen-binding fragment thereof.

13. An immunoconjugate, comprising the anti-CD40 antibody or the antigen-binding fragment according to claim 1, and other substances, wherein the other substances comprises a cytotoxic agent.

14. A pharmaceutical composition, comprising the anti-CD40 antibody or the antigen-binding fragment according to claim 1 or an immunoconjugate, and optionally a pharmaceutical adjuvant material, wherein the immunoconjugate comprises the anti-CD40 antibody or the antigen-binding fragment, and other substances, wherein the other substances comprises a cytotoxic agent.

15. A pharmaceutical composition, comprising the anti-CD40 antibody or the antigen-binding fragment according to claim 1 or an immunoconjugate, and other therapeutic agents, and optionally a pharmaceutical adjuvant material; wherein the other therapeutic agents are selected from a chemotherapeutic agent, antibodies and a cytotoxic agent, wherein the immunoconjugate comprises the anti-CD40 antibody or the antigen-binding fragment, and other substances, wherein the other substances comprises a cytotoxic agent.

16. A combined product, comprising the anti-CD40 antibody or the antigen-binding fragment according to claim 1 or an immunoconjugate, and one or more therapeutic agents, wherein the one or more therapeutic agents comprises a chemotherapeutic agent, a cytotoxic agent and antibodies, wherein the immunoconjugate comprises the anti-CD40 antibody or the antigen-binding fragment, and other substances, wherein the other substances comprises a cytotoxic agent.

17. Use of the anti-CD40 antibody or the antigen-binding fragment according to claim 1 or an immunoconjugate in the preparation of a drug for preventing or treating a tumor or an infectious disease in a subject wherein the immunoconjugate comprises the anti-CD40 antibody or the antigen-binding fragment, and other substances, wherein the other substances comprises a cytotoic agent.

18. The use according to claim 17, wherein the tumor is a cancer, wherein the cancer comprises lymphoma, breast cancer, liver cancer, lung cancer, myeloma, leukemia, skin cancer, head and neck cancer, myelodysplastic syndrome, bladder cancer, pancreatic cancer, renal carcinoma, and colon cancer; and the infectious disease is a bacterial infection, a viral infection, a fungal infection or a protozoal infection.

19. A kit for detecting CD40 in a sample, comprising the anti-CD40 antibody or the antigen-binding fragment according to claim 1, for use in performing the steps of:
(a) contacting the sample with the anti-CD40 antibody or the antigen-binding fragment; and
(b) detecting the formation of a complex of the anti-CD40 antibody or the antigen-binding fragment and CD40; optionally, the anti-CD40 antibody or the antigen-binding fragment is detectably labeled.

20. A host cell containing the vector according to claim 10, wherein the host cell is selected from an E. coli cell, a yeast cell, a mammalian cell or other cells suitable for the preparation of an antibody or an antigen-binding fragment thereof.
